(19) United States
(12) Patent Application Publication
(10) Pub. No.: US 2017/0102375 A1
(43) Pub. Date: Apr. 13, 2017

(54) METHODS FOR DETERMINING THE EFFECTS OF COMPOUNDS ON JAK/STAT ACTIVITY

(71) Applicant: Nodality, Inc., South San Francisco, CA (US)

(72) Inventors: Wendy J. Fantl, San Francisco, CA (US); Alessandra Cesano, Redwood City, CA (US); Todd Covey, San Carlos, CA (US)

(21) Appl. No.: 15/388,658

(22) Filed: Dec. 22, 2016

Related U.S. Application Data
(63) Continuation of application No. 15/179,679, filed on Jun. 10, 2016, now abandoned.
Application No. 15/179,679 is a continuation of application No. 14/294,592, filed on Jun. 3, 2014, now abandoned.
Application No. 14/294,592 is a continuation of application No. 13/913,029, filed on Jun. 7, 2013, now abandoned.
Application No. 13/913,029 is a continuation of application No. 12/687,873, filed on Jan. 14, 2010, now abandoned.
(60) Provisional application No. 61/144,684, filed on Jan. 14, 2009.
(60) Provisional application No. 61/170,348, filed on Apr. 17, 2009.
(60) Provisional application No. 61/182,518, filed on May 29, 2009.
(60) Provisional application No. 61/218,718, filed on Jun. 19, 2009.
(60) Provisional application No. 61/226,878, filed on Jul. 20, 2009.

(51) Int. Cl. G01N 33/50; C12Q 1/68

(52) U.S. Cl. CPC G01N 33/5041; G01N 33/5073; C12Q 1/6883; C12Q 2600/154; C12Q 2600/178; C12Q 2600/106

(57) ABSTRACT

An embodiment of the present invention is a method for subjecting a hematopoetic cell to a JAK/STAT inhibitor, determining the activity of gain-of-function mutations of a Jak family kinase, determining the expression levels and activity of JAK/STAT regulatory proteins, correlating the expression levels and the activity of JAK/STAT regulatory proteins with the activity of gain-of-function mutations of a Jak family kinase and with a response to the JAK/STAT inhibitor, and then classifying the cells. A further embodiment of the invention includes determining the clinical outcome based on the cell classification, determining a method of treatment, determining dosing and scheduling of at least one of the JAK/STAT inhibitors or other compounds.

### CROSS-REFERENCE

This application is a continuation of U.S. patent application Ser. No. 15/179,679, filed on Jun. 10, 2016, which is a continuation of U.S. patent application Ser. No. 14/294,592, filed on Jun. 3, 2014, which is a continuation of U.S. patent application Ser. No. 13/913,029, filed on Jun. 7, 2013, which is a continuation of U.S. patent application Ser. No. 12/687,873, filed on Jan. 14, 2010, which claims the benefit of U.S. Provisional Patent Application Nos. 61/144,684, filed on Jan. 14, 2009, 61/170,348, filed on Apr. 17, 2009, 61/182,518, filed on May 29, 2009, 61/218,718, filed on Jun. 19, 2009, and 61/226,878, filed on Jul. 20, 2009, each of which applications is incorporated herein by reference.

### BACKGROUND OF THE INVENTION

Many conditions are characterized by disruptions of cellular pathways that lead, for example, to aberrant control of cellular processes, with uncontrolled growth and increased cell survival. These disruptions are often caused by changes in the activity of molecules participating in cellular pathways. For example, alterations in specific signaling pathways have been described for many cancers.

Elucidation of the signal-transduction networks that drive neoplastic transformation in both solid tumors and hematological malignancies has led to rationally designed cancer therapeutics that target signaling molecules. Many of the signaling molecules that are targeted are kinases. Recently, several groups discovered a recurrent mutation in the Janus Kinase 2 (Jak2) tyrosine kinase that is present in most patients with polycythaemia vera (PV), essential thrombocythaemia (ET), and primary myelofibrosis. As a result, drug companies are currently developing drugs to inhibit JAK/STAT pathway activity.

Accordingly, there is a need to look at cell populations to determine what signaling events may contribute to their responses to compounds.

### SUMMARY OF THE INVENTION

In some embodiments, the invention is a method of analyzing the effect of a compound comprising: contacting a cell of interest with a compound of interest; analyzing activity of a gain-of-function mutation of a JAK/STAT pathway component in said cell; analyzing activity of a JAK/STAT regulatory protein in said cell; and correlating the activity of the JAK/STAT regulatory protein with the activity of the JAK/STAT pathway component.

In some embodiments, the invention is a method for analyzing the effect of a compound on a cell comprising: subjecting a hematopoetic cell to a plurality of compounds, whereby one such compound may be a JAK/STAT inhibitor, specifically a Jak2 inhibitor as an example; determining the activity of gain-of-function mutations of JAK kinases by determining the phosphorylation status of that JAK kinase and determining the phosphorylation status of at least one of a plurality of JAK kinase substrates comprising phospho-amino acid residues on the JAK kinase, phospho-amino acid residues on cytokine receptors that engage the JAK kinase, phospho-amino acid residues on Stats, and on a plurality of signaling molecules in parallel or downstream of Jak2; determining the expression levels and activity of JAK/STAT regulatory proteins, such as SOCS3, Lnk, or SH2-B, correlating the expression levels and the activity of JAK kinase regulatory proteins with the activity of gain-of-function mutations of the JAK kinase and with a response to the compound; and then classifying the cells. A further embodiment of the invention includes determining the clinical outcome based on the cell classification. A further embodiment includes determining a method of treatment. A further embodiment includes a method for determining the potency, selectivity, and off-target effects of a compound or combination of compounds in a physiological relevant setting, for example whole blood samples. Additionally, this method may be used to analyze drug effects in other tissues if subsets of the cells being analyzed can serve as surrogates for cells in other tissues. For example, gated T cells in whole blood may serve as surrogates for tumor cells for some cellular processes. In some embodiments, this method may be used to determine dosing, and to characterize the function of compounds in drug screening, preclinical studies, and phase 1 and phase 2 clinical trials. In some embodiments, this method may be used to select the dosing and scheduling of a therapeutic compound or combination of compounds in an individual patient, based on profiles of single cell signaling in the patient's own cells.

In one embodiment of the present invention, the compound is a modulator (also called a stim or stimulator in some instances). The modulator may be selected from the group of growth factors, cytokines, adhesion molecule modulators, hormones, small molecules, polynucleotides, antibodies, natural compounds, lactones, chemotherapeutic agents, immune modulators, carbohydrates, proteases, ions, reactive oxygen species, or radiation. The method may analyze the activatable elements after subjecting the cell to the modulator as well as determining the activity of gain-of-function mutations of JAK/STAT pathway components with Jak2 as an example, determining the expression levels and activity of JAK/STAT regulatory proteins, correlating the expression levels and the activity of JAK/STAT regulatory proteins with the activity of gain-of-function mutations of JAK/STAT pathway components (for example, Jak 2) and with a response to the compound, and then classifying the cells.

One embodiment of the present invention comprises subjecting a hematopoietic cell to a plurality of compounds, whereby one such compound may be a JAK/STAT inhibitor, and determining the activity of gain-of-function mutations in cytokine receptors, determining epigenetic changes, such as methylation or acetylation, determining microRNA changes, determining expression levels and activity of JAK/STAT regulatory proteins, correlating the expression levels and activity of the JAK/STAT regulatory proteins with the activity of gain-of-function mutations in the cytokine receptors, the epigenetic changes, and the microRNA changes, and then correlating the results of the analysis with the response to the JAK/STAT inhibitor and classifying the cells. The inhibitor may be direct or indirect, acting on Jak2 for example, or on upstream, downstream or parallel components of the JAK/STAT signaling pathway. A further embodiment of the invention includes determining the clinical outcome based on the cell classification. A further embodiment of the invention includes comparing the phenotypes of cells within a population, for example in mixed populations of healthy and disease cells. A further embodiment of the invention includes identifying rare cells within a population, and identifying the effects of modulators or compounds upon these rare cells. A further embodiment includes determining a method of treatment. A further embodiment includes determining dosing and scheduling of at least one of the compounds, such as a JAK/STAT inhibitor.

In each instance where a gain-of-function mutation can be analyzed, the gain-of-function mutation can be replaced with a loss-of-function mutation.

### BRIEF DESCRIPTION OF THE DRAWINGS

FIG. 1 shows the use of phosphoflow to distinguish cell types in a heterogeneous population and simultaneously measure pathway inhibition.

FIGS. 2A-B shows the use of phosphoflow to identify pathway-selective inhibitors in B-cells gated from a peripheral blood mononucleated cell (PBMC) sample. FIG. 2A shows p-AKT levels after treatment with a compound inhibitor generated in B-cells derived from PBMCs population. FIG. 2B shows the log 10 (IC50) levels generated from random samples.

FIGS. 3A-C shows simultaneous measurements of drug potency on three kinase targets in B-cells, T-cells and non-B-cells/T-cells. FIG. 3A shows the kinase target p-BLNK, FIG. 3B shows the kinase target p-AKT, and FIG. 3C shows the kinase target p-Erk.

FIG. 4 shows simultaneous measurement of IL-27 signaling within distinct cell types of the same AML bone marrow sample.

FIGS. 5A-D shows the use of phosphoflow to reveal differential responses to cytokine signaling within distinct cell sub-populations. FIG. 5A shows the assay protocol. FIG. 5B shows fold-change of pStat-1expression levels in various blood cells after stimulation with GM-CSF. FIG. 5C shows fold-change of pStat-1expression levels in various blood cells after stimulation with IFN-alpha. FIG. 5D shows fold-change of pStat-1expression levels in various blood cells after stimulation with IL-2.

FIG. 6 shows combinations of cell-specific modulators to assess selectivity as well as potency.

FIG. 7 shows compound profiling using combo stims (a combination of stimulations or modulators).

FIGS. 8A-C shows the use of phosphoflow to assess the specificity of a compound: FIG. 8A shows the assay protocol whole blood is treated with the compound JAK3 Inhibitor VI, labeled using a cocktail of fluorochrome-conjugated antibodies designed to recognize specific cell types and p-STAT signaling molecules, and analyzed using multiparameter phosphoflow. FIG. 8B shows the cell signaling pathways stimulated or affected by the protocol. FIG. 8C shows that the assay protocol can reveal that different cell types have different sensitivity to the JAK3 Inhibitor VI compound.

FIG. 9 shows that using phosphoflow to compare myeloid cells in healthy and AML patients identifies a correlation between the disease state and the phosphorylation state of Stat-3 and Stat-5.

FIGS. 10A-C shows the use of phosphoflow to monitor the effects of drug treatment on patients, including the development of drug resistance: patient samples taken at diagnosis and after therapy are evaluated for G-CSF signaling using multiparameter phosphoflow and exhibit different profiles of p-Stat1, p-Stat3 and p-Stat5 activation. FIG. 10A shows p-Stat1 vs. p-Stat3 profiles in basal (unstimulated) and G-CSF stimulated cells. FIG. 10B shows p-Stat1 vs. p-Stat5 profiles in basal (unstimulated) and G-CSF stimulated cells. FIG. 10C shows p-Stat1 vs. p-Stat3 profiles in basal (unstimulated) and G-CSF stimulated cells.

FIG. 11 shows the use phosphoflow profiling to survey compounds (listed in Table 8) that affect JAK/STAT activity in blood cells stimulated with GM-CSF, CD40L, and IL-2 to activate multiple signaling pathways in monocytes, B-cells, and T-cells, respectively. FIG. 11A shows the various cell types used in the phosphoflow profiling. FIG. 11B shows the various phosphoflow profiling of the cells using various antibodies. FIG. 11C shows the determination of cell subset specific IC50 for particular compounds.

FIGS. 12A-B shows that multiparameter phosphoflow reveals that differences in cellular environment (PBMCs versus Whole Blood) affect the potency of the compounds listed in Table 8, as measured by their effects of p-STAT5 levels in T cells. FIG. 12A shows the affect of various compounds on p-STAT5 levels (% of control) in T-cells derived from the PBMCs population and their determined IC50 levels for particular compounds. FIG. 12B shows the affect of various compounds on p-STAT5 levels (% of control) in T-cells derived from the Whole Blood and their determined IC50 levels for particular compounds.

FIGS. 13A-C shows the use of multiparameter phosphoflow to compare the specificity of the JAK/STAT inhibitor compounds (listed in Table 8) by measuring pSTAT5 in stimulated T cells and monocytes. FIG. 13A shows phosphoflow profiling of p-STAT5 levels (% of control) in T-cells derived from whole blood. FIG. 13B shows phosphoflow profiling of p-STAT5 levels (% of control) in monocytes derived from whole blood. FIG. 13C shows how the phosphoflow profiling of T-cells and monocytes can be used to establish a target exposure for in vivo studies.

FIG. 14 shows that potency measurements the JAK/STAT inhibitor CP-690550 using multiparameter phosphoflow would predict an optimal drug dose comparable to the target drug dose determined by a clinical trial.

FIGS. 15A-B shows the use of a single multiparameter phosphoflow assay to measure the potency and selectivity of the JAK/STAT inhibitor compounds listed in Table 8. FIG. 15A shows phosphoflow profiling of p-STAT5 levels (% of control) in T-cells derived from PBMCs population and determination of the IC50 for particular compounds. FIG. 15B shows phosphoflow profiling of p-STAT5 levels (% of control) in monocytes derived from PBMCs population and determination of the IC50 for particular compounds.

FIGS. 16A-B shows the use of multiparameter phosphoflow to monitor off-target activities of JAK/STAT inhibitor compounds listed in Table 8; specifically, off-target inhibition and induction of ERK signaling. FIG. 16A shows phosphoflow profiling of p-ERK levels (% of control) stimulated by GM-CSF in monocytes derived from PBMCs population and determination of the IC50 for particular compounds. FIG. 16B shows phosphoflow profiling of p-ERK levels (% of control) stimulated by CD40L in B-cells derived from PBMCs population and determination of the IC50 for particular compounds.

FIG. 17 shows the use of multiparameter phosphoflow to monitor off-target activities of JAK/STAT inhibitor compounds; specifically, off-target inhibition of NFkB signaling and determination of the IC50 for a particular compound.

FIG. 18 shows an example of how different cell subsets can be gated based on expression of phenotypic surface markers. Cell subsets were identified and gated on the basis of relative expression of surface markers.

FIG. 19 shows the responses of three cell subsets from three different patient donors to modulation with IL-27 and G-CSF. Cell subsets from different patient donors responded differently to modulation with IL-27 and G-CSF.

FIG. 20 shows that the JAK/STAT inhibitor CP-690550 could inhibit the p-Stat readout completely at the 333 nM concentration point in cells of patients having IL-27-induced signaling above basal levels where cells were incubated with four different doses of CP-690550 (0 nM, 33 nM, 333 nM, 3333 nM) prior to modulation with IL-27.

FIG. 21 shows that the JAK/STAT inhibitor CP-690550 could inhibit the p-Stat readout completely at the 3333 nM concentration point in cells of patients having G-CSF-induced signaling above basal levels where cells were incubated with four different doses of CP-690550 (0 nM, 33 nM, 333 nM, 3333 nM) prior to modulation with G-CSF.

FIG. 22 shows several uses for single cell network profiling (SCNP) in the development of a drug compound.

FIG. 23 shows how single cell network profiling can take simultaneous measurements and advantages associated with SCNP.

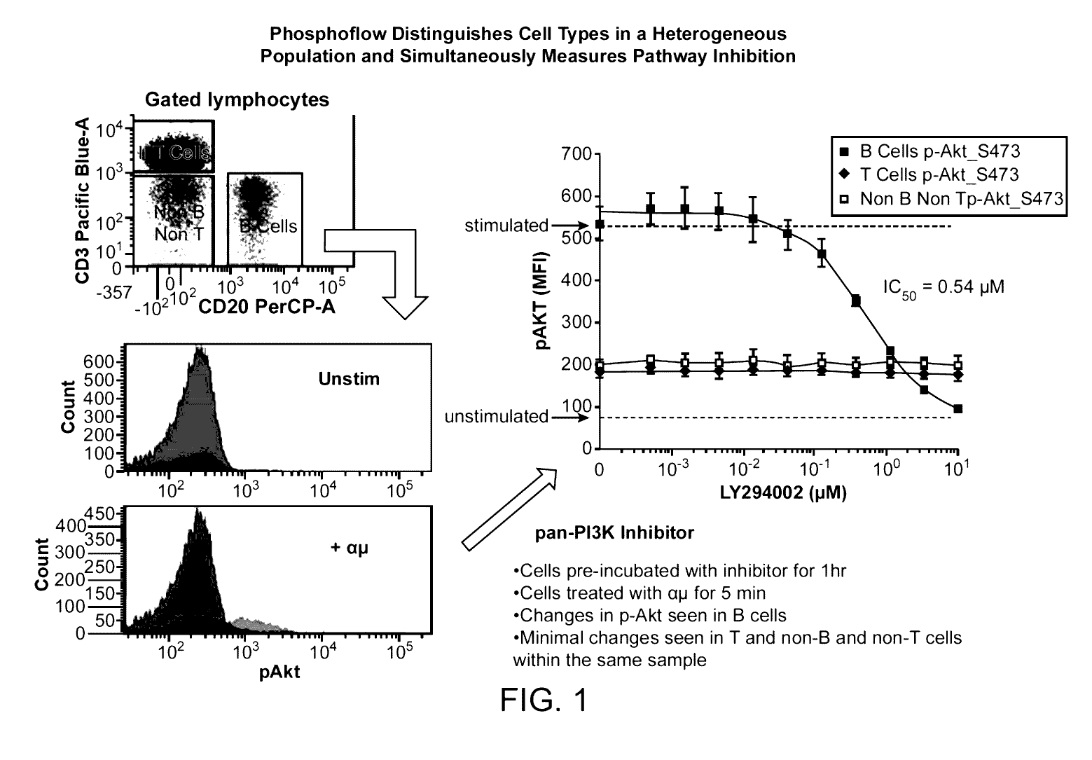

### DETAILED DESCRIPTION OF THE INVENTION

The present invention incorporates information disclosed in other applications and texts. The following publications are hereby incorporated by reference in their entireties: Haskell et al, Cancer Treatment, 5^{th} Ed., W.B. Saunders and Co., 2001; Alberts et al., The Cell, 4^{th} Ed., Garland Science, 2002; Vogelstein and Kinzler, The Genetic Basis of Human Cancer, 2d Ed., McGraw Hill, 2002; Michael, Biochemical Pathways, John Wiley and Sons, 1999; Weinberg, The Biology of Cancer, 2007; Immunobiology, Janeway et al. 7^{th} Ed., Garland, and Leroith and Bondy, Growth Factors and Cytokines in Health and Disease, A Multi Volume Treatise, Volumes 1A and 1B, Growth Factors, 1996; and Immunophenotyping, Chapter 9: Use of Multiparameter Flow Cytometry and Immunophenotyping for the Diagnosis and Classfication of Acute Myeloid Leukemia, Stelzer, et al., Wiley, 2000.

Patents and applications that are also incorporated by reference include U.S. Pat. Nos. 7,381,535 and 7,393,656 and U.S. patent application Ser. Nos. 10/193,462; 11/655,785; 11/655,789; 11/655,821; 11/338,957, 61/048,886; 61/048,920; and 61/048,657.

Some commercial reagents, protocols, software and instruments that are useful in some embodiments of the present invention are available at the Becton Dickinson Website http://www.bdbiosciences.com/features/products/, and the Beckman Coulter website, http://www.beckmancoulter.com/Default.asp?bhfv=7.

Relevant articles include: Krutzik et al., High-content single-cell drug screening with phosphospecific flow cytometry, Nat. Chem. Biol., Dec. 23, 2007, 4(2): 132-142; Irish et al., Flt3 Y591 duplication and Bcl-2 over expression are detected in acute myeloid leukemia cells with high levels of phosphorylated wild-type p53, Blood, Mar. 15, 2007, 109(6): 2589-96; Irish et al. Mapping normal and cancer cell signaling networks: towards single-cell proteomics, Nat. Rev. Cancer, February 2006, 6(2): 146-155; Irish et al., Single cell profiling of potentiated phospho-protein networks in cancer cells, Cell, Jul. 23, 2004, 118(2): 217-228; Schulz, K. R., et al., Single-cell phospho-protein analysis by flow cytometry, Curr. Protoc. Immunol., August 2007, 78:8 8.17.1-20; Krutzik, P. O., et al., Coordinate analysis of murine immune cell surface markers and intracellular phosphoproteins by flow cytometry, J. Immunol., Aug. 15, 2005, 175(4): 2357-65; Krutzik, P. O., et al., Characterization of the murine immunological signaling network with phosphospecific flow cytometry, J Immunol., Aug. 15, 2005, 175(4): 2366-73; Shulz et al., Curr. Prot. Immun., 2007, 78:8.17.1-20; Krutzik, P. O. and Nolan, G. P., Intracellular phospho-protein staining techniques for flow cytometry: monitoring single cell signaling events, Cytometry A, Sep. 17, 2003, 55(2): 61-70; Hanahan D., Weinberg, The Hallmarks of Cancer, Cell, Jan. 7, 2000, 100(1): 57-70; and Krutzik et al, High content single cell drug screening with phosphospecific flow cytometry, Nat. Chem. Biol., February 2008, 4(2): 132-42. Experimental and process protocols and other helpful information can be found at http://proteomics.stanford.edu. The articles and other references cited below are also incorporated by reference in their entireties for all purposes.

Other relevant articles include: Vannucchi et al., Clinical correlates of Jak2 V617F presence or allele burden in myeloproliferative neoplasms: a critical reappraisal, Leukemia, May 22, 2008, 22: 1299-1307; Gueller et al., Adaptor protein Lnk associates with Y568 in c-Kit, Biochemical Journal. Jun. 30, 2008, manuscript; Tong et al., Lnk inhibits erythropoiesis and Epo-dependent Jak2 activation and downstream signaling pathways, Hematopoiesis. Jun. 15, 2005, 105 (12): 4604-4612; Bersenev et al., Lnk controls mouse hematopoietic stem cell self-renewal and quiescence through direct interactions with Jak2, J. Clin. Invest., August, 2008, 118(8): 2832-2844; Levine et al., Role of Jak2 in the pathogenesis and therapy of myeloproliferative disorders, Nat. Rev. Cancer, September 2007, 7: 673-683; Hookham et al., The myeloproliferative disorder-associated Jak2 V617F mutant escapes negative regulation by suppressor of cytokine signaling 3, Blood, Jun. 1, 2007, 109(11): 4924-4929; Koppikar, P. and Levine, R. L., Jak2 and MPL Mutations in Myeloproliferative Neoplasms, Acta Haematol., Jun. 20, 2008, 119: 218-225; and Zhang, C. C. and Lodish, H. F., Cytokines regulating hematopoietic stem cell function, Current Opinion in Hematology, July 2008, 15(4): 307-311.

The discussion below describes some of the preferred embodiments with respect to particular diseases. However, it should be appreciated that the principles may be useful for the analysis of many other diseases as well.

### General Methods

The following will discuss research and diagnostic methods, instruments, reagents, kits, and the biology involved with Myeloproliferative Neoplasms (MPNs) and other diseases. One aspect of the invention involves subjecting one or more cells to one or more of a plurality of compounds; analyzing the following states or nodes using techniques known in the art of phosphoflow cytometry, where individual cells are simultaneously analyzed for multiple characteristics, such as those selected from: activity of gain-of-function mutations in the JAK/STAT pathway (with mutations in Jak2 as an example), expression levels and activity of JAK/STAT regulatory proteins, phosphorylation status of JAK kinase and various JAK kinase substrates, activity of gain-of-function mutations of cytokine receptors, epigenetic changes, post-translational modifications of JAK kinases (with Jak2 as an example) and JAK kinase regulatory proteins, microRNA changes, and activity and expression of Jak2; correlating the results of the analysis with a response to a compound; and classifying said cells into clinical outcomes. Alternatively, one aspect of the invention involves analyzing the effect of a compound on a cell of interest by analyzing activity of a gain-of-function mutation of a JAK/STAT pathway component in the cell. The methods of the invention can also be used to analyze loss-of-function mutations of a JAK/STAT pathway component. In another embodiment, the method of the invention analyzes activity of a gain-of-function mutation of a JAK/STAT pathway component in the cell, as well as activity of a JAK/STAT regulatory protein in the cell. Analysis of both a gain-of-function mutation of a JAK/STAT pathway component and a JAK/STAT regulatory protein allow for correlation to remove artifacts caused by factors unrelated to alteration in the signaling pathway. In another embodiment, the methods described can further analyze the expression level of the JAK/STAT regulatory protein.

In some embodiments, the present invention includes methods for validating candidate nodes in a signaling network. Node validation may include determining which signaling activities a given node may report on, and determining optimal methods for identifying the activation state of that node. Multiple receptors and ligands converge upon the JAK/STAT pathway, making node validation important for understanding the signaling mechanism that is measured for any given node. See Table 10 for examples of receptors and ligands that converge on the JAK/STAT pathway. In some embodiments, node validation can comprise the following steps:
1) Identify the pathway in which the node participates.
2) Identify the receptor and upstream activators of the node.
3) Identify cell lines for optimal detection of node states. This step can include measuring expression levels of the receptor or receptors in one or more cell lines.
4) Identify one or more extracellular modulators that activate the node (in the preferred embodiment, one (1) to three (3) modulators are generally selected.
5) Validate fluorochrome-conjugated antibodies from different vendors for detecting activated node states. If flurochrome-conjugated primary antibodies are not available, flurochrome-conjugated secondary antibodies can be used.
6) Perform titrations of modulators and antibodies in cell lines and primary cells, for example peripheral blood mononuclear cells (PBMCs) or BMMCs.
7) Perform kinetic studies to identify optimal conditions for detecting node activation.
8) Perform control experiments to determine the specificity of the primary antibody. For example, one sample of phospho-specific antibody may be pre-incubated with phospho-peptide epitopes to inhibit the epitope-specific binding sites and then contact with cells. Specificity for the target epitope can be determined by comparing fluorescence of cells contacted with pre-incubated “bound” antibody to that of cells contacted with unbound antibody that was not incubated with peptide. Another sample of phospho-specific antibody may be pre-incubated with non-phospho-peptide epitopes and then contacted with cells to determine specificity of binding to the phosphorylated epitope.

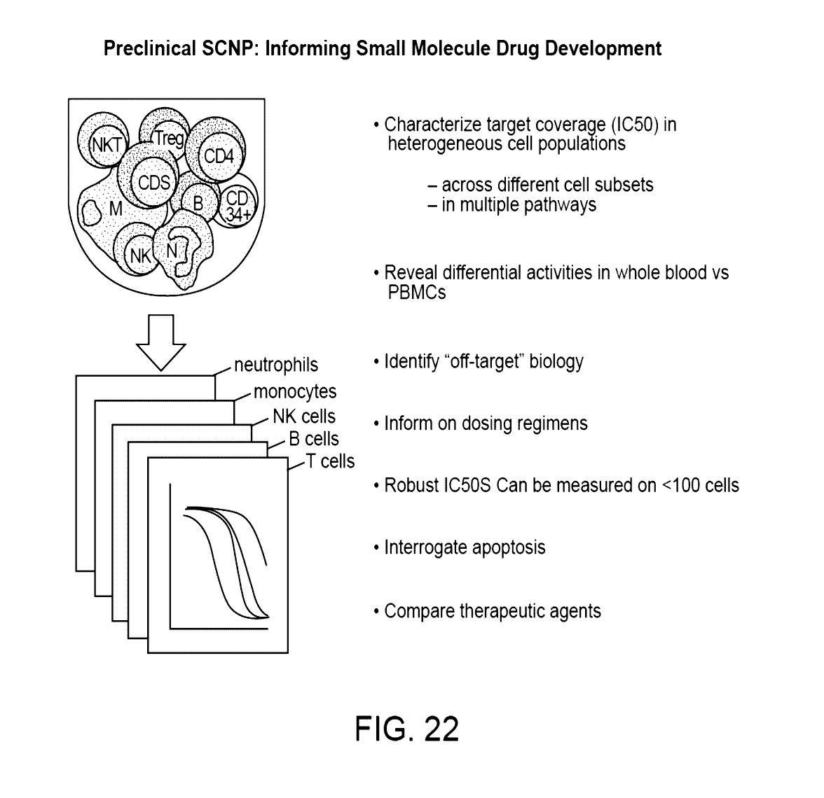

In some embodiments, the present invention is directed to selection of at least one of a plurality of compounds for optimization and preclinical studies. In some embodiments, the present invention is directed to determining dosing and scheduling of at least one of a plurality of compounds that correct the clinical outcome. In some embodiments, the invention employs techniques including but not limited to, flow cytometry, cellular imaging, mass spectrometry, mass spectrometry-based flow cytometry, nucleic acid microarrays, or other cell-based functional assays in which to determine the concentration curves and the derived IC₅₀ values for target inhibition for one or more of a plurality of compounds against one or more intracellular signalling pathways in cells including but not limited to, cell lines, cell sub-sets delineated by phenotypic markers within complex primary samples. Examples of uses of the methods of the present invention are described in FIG. 22, as applied to drug development and screening.

In some embodiments, the invention is directed to methods for determining the activation level of one or more activatable elements in a cell upon treatment with one or more modulators. The activation of an activatable element in the cell upon treatment with one or more modulators can reveal operative pathways in a condition that can then be used, e.g., as an indicator to predict course of the condition, identify risk group, predict an increased risk of developing secondary complications, choose a therapy for an individual, predict response to a therapy for an individual, determine the efficacy of a therapy in an individual, and determine the clinical outcome for an individual.

In some embodiments, the invention is directed to methods for classifying a cell by contacting the cell with a compound, such as a JAK/STAT inhibitor, determining the presence or absence of an increase in activation level of an activatable element in the cell, and classifying the cell based on the presence or absence of the increase in the activation of the activatable element. The inhibitor may be direct or indirect, acting directly on a JAK/STAT pathway component, for example Jak2 kinase, or on upstream, downstream, or parallel regulators of the JAK/STAT signaling pathway. In some embodiments, the invention is directed to methods of determining the presence or absence of a condition in an individual by subjecting a cell from the individual to a modulator, determining the activation level of an activatable element in the cell, and determining the presence or absence of the condition based on the activation level upon treatment with a modulator. In some embodiments, the invention is directed to methods of determining the presence or absence of a condition in an individual by subjecting a cell from the individual to a modulator and an inhibitor, determining the activation level of an activatable element in the cell, and determining the presence or absence of the condition based on the activation level upon treatment with a modulator and an inhibitor.

In some embodiments, the invention is directed to methods of determining a phenotypic profile of a population of cells by exposing the population of cells to one or more (a plurality of) modulators in separate cultures, wherein at least one of the modulators is an inhibitor, determining the presence or absence of an increase in activation level of an activatable element in the cell population from each separate culture and classifying the cell population based on the presence or absence of the increase in the activation of the activatable element from each separate culture.

In some embodiments, the present invention is a method for drug screening, diagnosis, prognosis and prediction of disease treatment. Reports generated by the present invention may be used to measure signaling pathway activity in single cells, identify signaling pathway disruptions in diseased cells, including rare cell populations, identify response and resistant biological profiles that guide the selection of therapeutic regimens, monitor the effects of therapeutic treatments on signaling in diseased cells, and monitor the effects of treatment over time. These reports can enable biology-driven patient management and drug development, improving patient outcome, reducing inefficient uses of resources, and improving the speed of drug development cycles.

The subject invention also provides kits for use in determining the physiological status of cells in a sample, the kit comprising one or more antibodies for detecting phosphorylated or non-phosphorylated epitopes of one or more (a plurality of) JAK/STAT inhibitors, modulators, fixatives, containers, plates, buffers, and can additionally comprise one or more therapeutic agents. The above reagents for the kit are all recited and listed in the present application. The kit can further comprise a software package for data analysis of the physiological status, which can include reference profiles for comparison with the test profile. The kit can also include instructions for use for any of the above applications. See the examples below for components of kits of the present invention.

One or more cells or cell types, or samples containing one or more cells or cell types, can be isolated from body samples. Cell types include, but are not limited to whole unfractionated blood, ficoll-purified-peripheral blood mononuclear cells (PBMCs), whole unfractionated bone marrow, ficoll-purified bone mononuclear cells. The cells can be separated from body samples by centrifugation, elutriation, density gradient separation, apheresis, affinity selection, panning, FACS, centrifugation with Hypaque, etc. By using antibodies specific for markers identified with particular cell types, a relatively homogeneous population of cells may be obtained. Alternatively, a heterogeneous cell population can be used.

Cells can also be separated by using filters. For example, whole blood can also be applied to filters that are engineered to contain pore sizes that select for the desired cell type or class. Rare pathogenic cells can be filtered out of diluted, whole blood following the lysis of red blood cells by using filters with pore sizes between 5 to 10 μm, as disclosed in U.S. patent application Ser. No. 09/790,673. Once a sample is obtained, it can be used directly, frozen, or maintained in appropriate culture medium for short periods of time. Methods to isolate one or more cells for use according to the methods of this invention are performed according to standard techniques and protocols well-established in the art. See also U.S. Patent Application Nos. 61/048,886; 61/048,920; and 61/048,657. See also, the commercial products from companies such as BD and BCI as identified above.

See also U.S. Pat. Nos. 7,381,535 and 7,393,656. All of the above patents and applications are incorporated by reference as stated above.

In some embodiments, the cells are cultured post collection in a media suitable for revealing the activation level of an activatable element (e.g. RPMI, DMEM) in the presence, or absence, of serum such as fetal bovine serum, bovine serum, human serum, porcine serum, horse serum, or goat serum. When serum is present in the media it could be present at a level ranging from 0.0001% to 30%.

Examples of hematopoietic cells include but are not limited to pluripotent hematopoietic stem cells, B-lymphocyte lineage progenitor or derived cells, T-lymphocyte lineage progenitor or derived cells, NK cell lineage progenitor or derived cells, granulocyte lineage progenitor or derived cells, monocyte lineage progenitor or derived cells, megakaryocyte lineage progenitor or derived cells and erythroid lineage progenitor or derived cells.

The term “patient” or “individual” as used herein includes humans as well as other mammals. The methods generally involve determining the status of an activatable element. The methods also involve determining the status of a plurality of activatable elements.

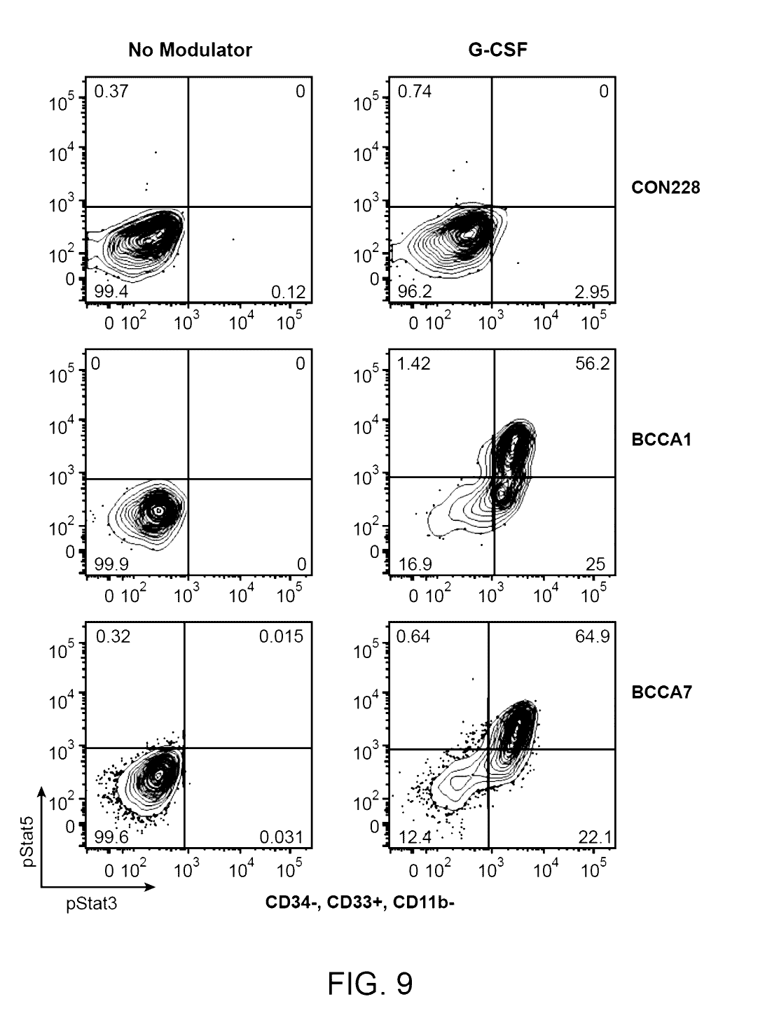

The classification of a cell according to the status of an activatable element can comprise classifying the cell as a cell that is correlated with a clinical outcome. In some embodiments, the clinical outcome is the prognosis and/or diagnosis of a condition. In some embodiments, the clinical outcome is the presence or absence of a neoplastic or a hematopoietic condition such as MPNs, acute leukemias, and myelodysplastic syndromes (MDSs). See U.S. Application No. 61/265,743, which is incorporated by reference. In some embodiments, comparisons between subsets of healthy cells and subsets of disease cells may reveal differences in the status of activatable elements which correlate with prognosis and/or diagnosis (See FIG. 9 for an example). These profiles of differences in activatable elements may be used to diagnose patients based on subsets of patient cells. In some embodiments, the clinical outcome is the staging or grading of a neoplastic or hematopoietic condition. Examples of staging include, but are not limited to, aggressive, indolent, benign, refractory, Roman Numeral staging, TNM Staging, Rai staging, Binet staging, WHO classification, FAB classification, IPSS score, WPSS score, limited stage, extensive stage, staging according to cellular markers, occult, including information that may inform on time to progression, progression free survival, overall survival, or event-free survival.

The analysis of a cell and the determination of the status of an activatable element can comprise classifying a cell as a cell that is correlated to a patient response to a treatment. In some embodiments, the patient response can be a complete response, partial response, nodular partial response, no response, progressive disease, stable disease and adverse reaction.

The classification of a rare cell according to the status of an activatable element can comprise classifying the cell as a cell that can be correlated with minimal residual disease or emerging resistance. See U.S. application Ser. No. 12/432,720, which is incorporated by reference.

The classification of a cell according to the status of an activatable element can comprise selecting a method of treatment. Examples of treatment methods include, but are not limited to, compounds that control some of the symptoms, such as aspirin and antihistamines, compounds that stimulate red blood cell production, such as erythropoietin or darbepoietin, compounds that reduce platelet production, such as hydroxyurea, anagrelide, and interferon-alpha, compounds that increase white blood cell production, such as G-CSF, chemotherapy, biological therapy, radiation therapy, phlebotomy, blood cell transfusion, bone marrow transplantation, peripheral stem cell transplantation, umbilical cord blood transplantation, autologous stem cell transplantation, allogeneic stem cell transplantation, syngeneic stem cell transplantation, surgery, induction therapy, maintenance therapy, and other therapy.

In some embodiments, cells (e.g. normal cells) other than the cells associated with a condition (e.g. cancer cells) or a combination of cells are used, e.g., in assigning a risk group, predicting an increased risk of relapse, predicting an increased risk of developing secondary complications, choosing a therapy for an individual, predicting response to a therapy for an individual, determining the efficacy of a therapy in an individual, and/or determining the prognosis for an individual. For example, in the case of cancer, infiltrating immune cells might determine the outcome of the disease. Alternatively, a combination of information from the cancer cell plus the immune cells in the blood that are responding to the disease, or reacting to the disease can be used for diagnosis or prognosis of the cancer.

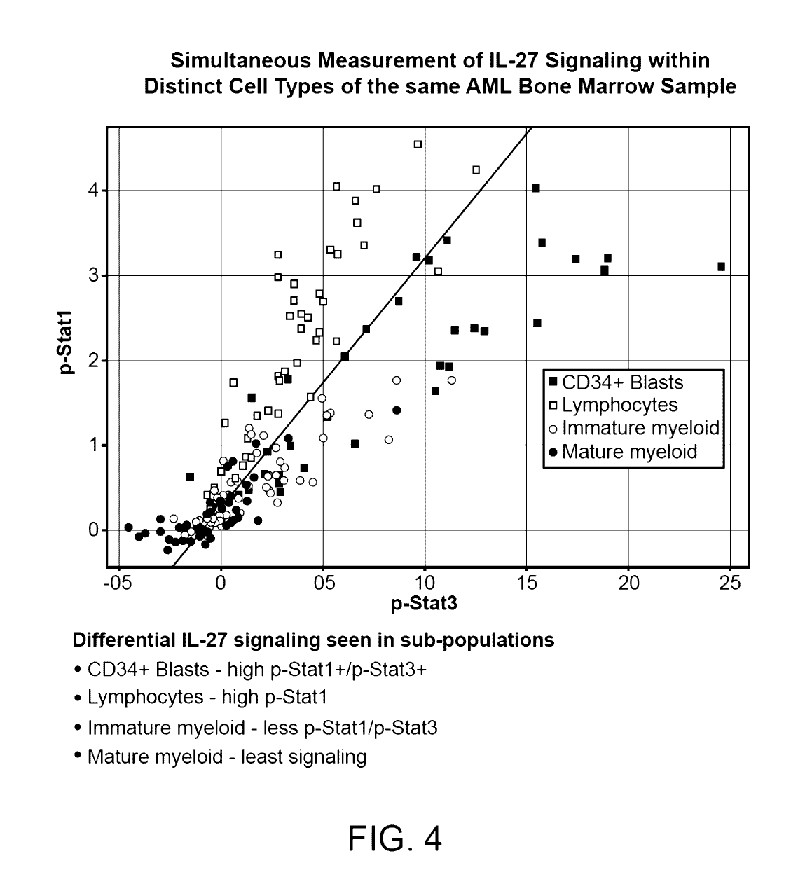

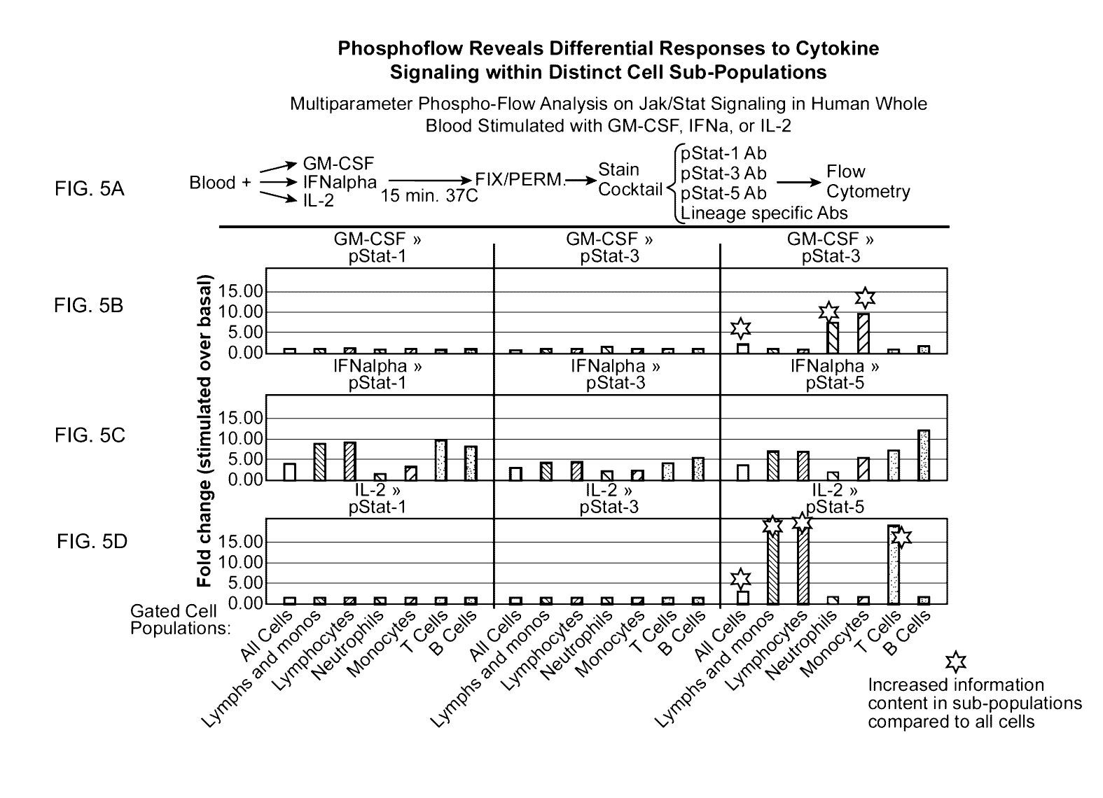

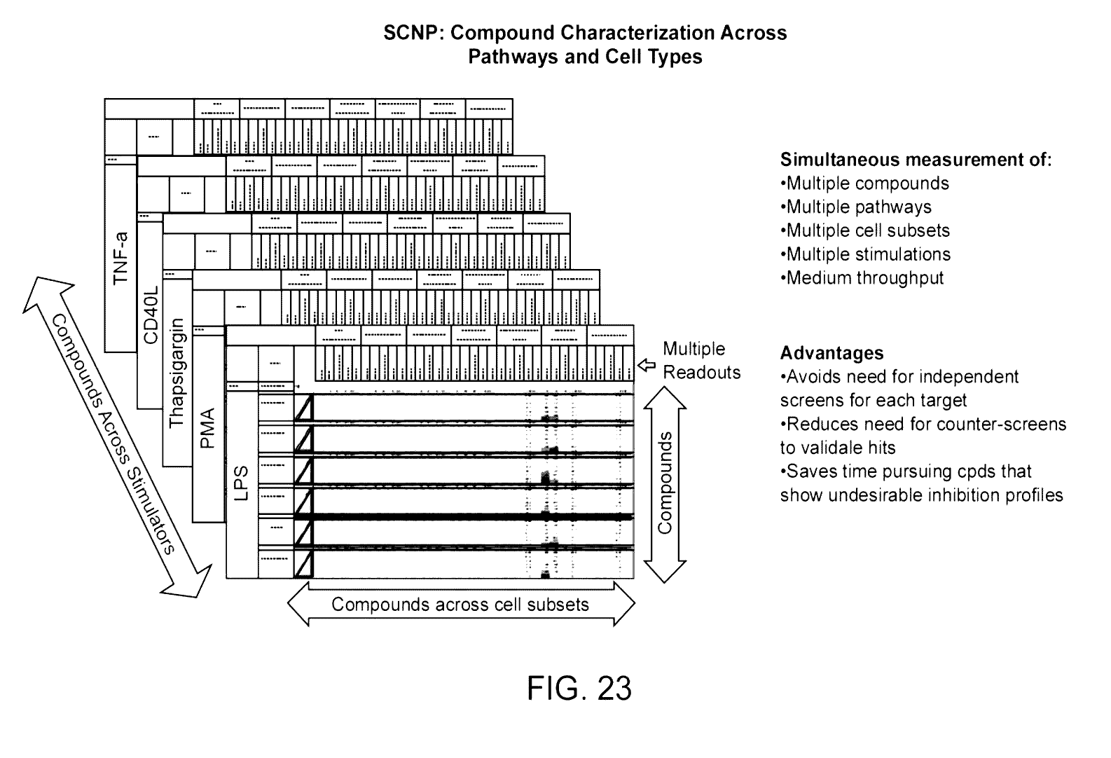

In some embodiments, the invention provides tools for the simultaneous measurement of multiple analytes in single cells within a complex mixture. The power of simultaneous measurement is also shown in FIG. 23. For example FIG. 4 shows how simultaneous measurements of IL-27 can be made in distinct cell types in a heterogeneous sample such as AML, patient bone marrow (For a review of IL-27-mediated signaling, see Colgan J, and Rothman, P., All in the family: IL-27 suppression of T(H)-17 cells. Nature Immunology 7: 899-901, 2006). Such tools can improve the efficiency of the drug discovery process and enable research on rare cell populations, such as cancer stem cells. The Cancer Stem Cell (CSC) hypothesis contends that, like normal tissue, cancers are maintained by a population of stem-like cells that exhibit the ability to self-renew as well as to differentiate into downstream non-self renewing progenitors and mature cells. For a review of the CSC hypothesis, see Wang J. C. and Dick J. E., Cancer stem cells: lessons from leukemia, Trends in Cell Biology, September 2008, 15(9) 494-501. The CSC hypothesis makes two predictions: 1) CSCs are required for tumor growth and metastasis 2) Elimination of CSCs is required for a cure. These predictions challenge investigators to isolate CSCs in all tumor types and identify the genes that regulate their function and response to conventional therapies. In some embodiments, the invention can detect rare cells within a population, with cancer stem cells as an example, and therefore can be used for diagnostic purposes or to examine the effects of compounds on these rare cells.

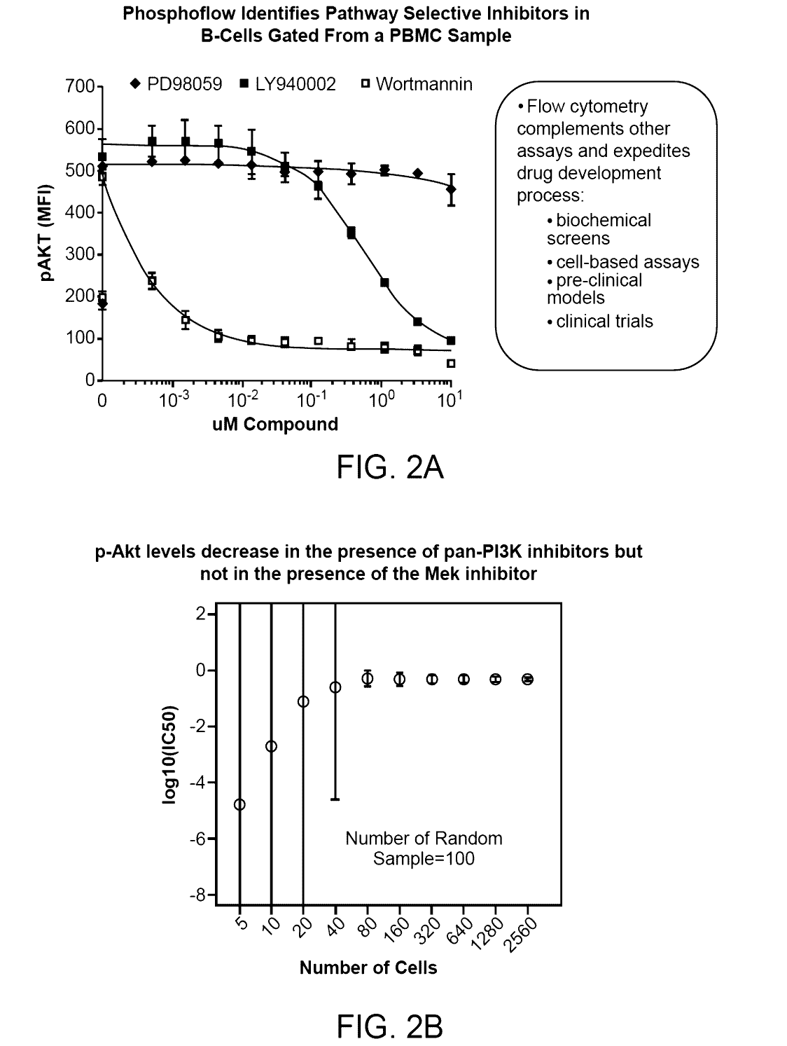

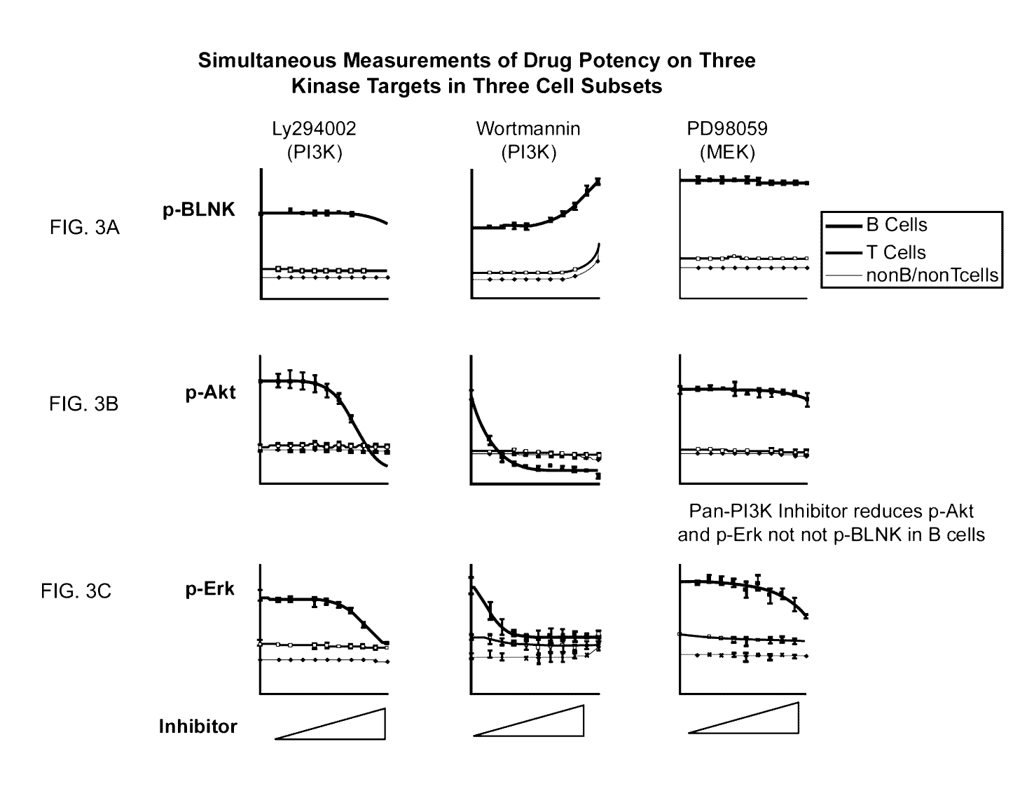

In some embodiments, the invention provides tools for making robust measurements of very small subpopulations of cells. For example, FIG. 2A shows the inhibition curves for different inhibitor compounds calculated based on evoked levels of pAKT (S473) in single cells after treatment with the inhibitor compound. The IC50 for LY940002 was calculated using pAKT measurements from 3,000 cells. A simulation shows that under these experimental conditions, measurements of fewer than 100 cells in a specific gated population can be used to determine an IC50 within a 95% confidence interval of 0.3 log units: At each concentration of the compound, the following quantities of cells were sampled from the 3,000 cell data set: 5, 10, 20, 40, 80, 160, 320, 640, 1280, and 2560. The median fluorescence index (MFI) was then computed only from these cells and used to estimate the IC50 value. This process was repeated 100 times at each sampling level to generate a list of IC50 values. If a small number of cells is sufficiently representative of the larger population, all the IC50 values are expected to be similar to each other, and therefore the 95% confidence interval will remain small. In this example, the 95% confidence interval IC50 remained within 0.3 log units for sample sizes of 80 cells and larger (See Table 9; See also FIG. 2B; error bars in FIG. 2B represent 2×SD). For samples of 40 cells and fewer, the IC50 became increasingly inconsistent. Depending on experimental conditions, such as cell type, nodes assayed, the percentage of cells that respond to the modulator, detection methods, and the strength of the signal, the minimal number of cells needed to obtain statistically relevant measurements may vary.

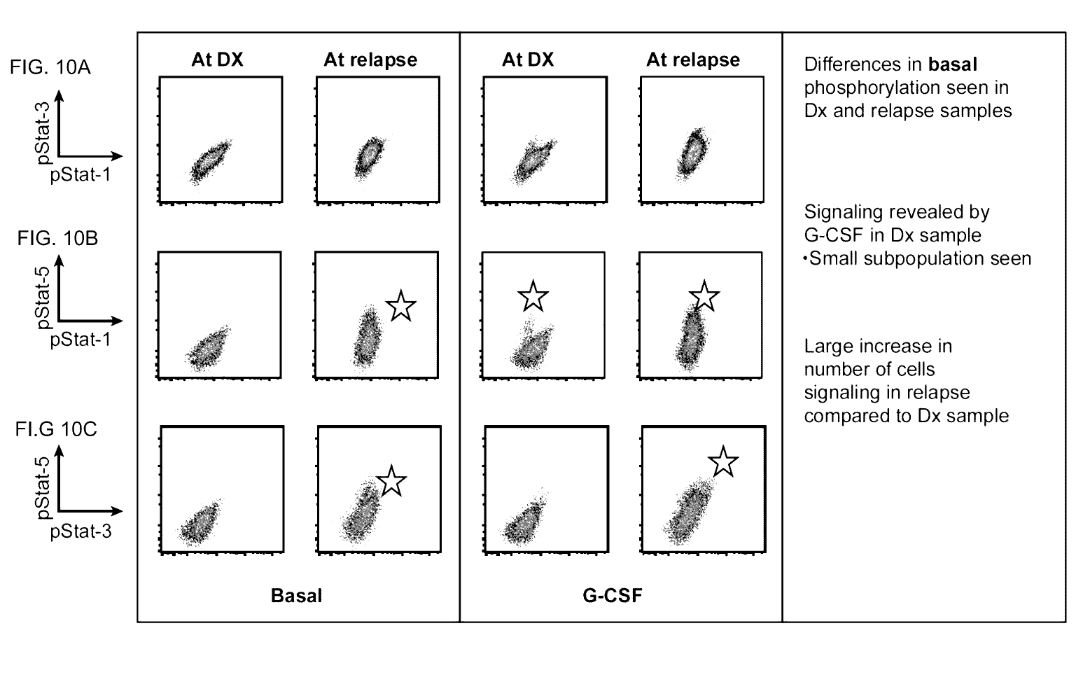

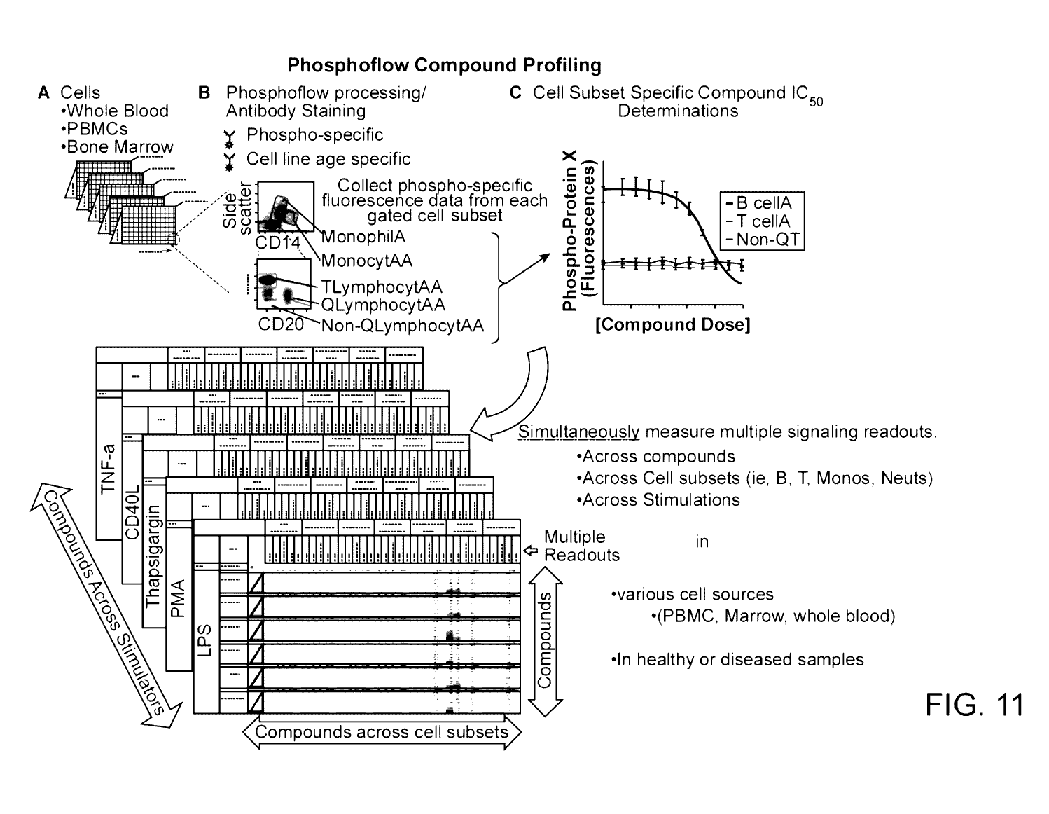

In some embodiments, the invention may be used to compare healthy cells and disease cells within the same population. In some embodiments, the invention can be used to detect rare cells within a population. For example, FIG. 10 shows basal levels of p-STAT1, p-STAT3 and p-STAT5 phosphorylation in a patient sample taken at diagnosis and relapse. There is a clear difference between the two samples. Activation of the JAK/STAT pathway by the myeloid cytokine G-CSF reveals signaling in a rare cell sub-set at diagnosis which seems to have grown out in the relapse sample. Patients in which evoked signaling is seen in a rare subpopulation at diagnosis could be candidates for JAK inhibitors in combination with the standard of care Ara-C-based regimens.

In some embodiments, the analysis involves working at multiple characteristics of the cell in parallel after contact with the compound. For example, the analysis can examine drug transporter function; drug transporter expression; drug metabolism; drug activation; cellular redox potential; signaling pathways; DNA damage repair; and apoptosis. Analysis can assess the ability of the cell to undergo the process of apoptosis after exposure to the experimental drug in an in vitro assay as well as how quickly the drug is exported out of the cell or metabolized.

In some embodiments, the methods of the invention provide methods for classifying a cell population or determining the presence or absence of a condition in an individual by subjecting a cell from the individual to a modulator and an inhibitor, determining the activation level of an activatable element in the cell, and determining the presence or absence of a condition based on the activation level. In some embodiments, the activation level of a plurality of activatable elements in the cell is determined. The inhibitor can be an inhibitor as described herein. In some embodiments, the inhibitor is a phosphatase inhibitor. In some embodiments, the inhibitor is H₂O₂. The modulator can be any modulator described herein. In some embodiments, the methods of the invention provides for methods for classifying a cell population by exposing the cell population to a plurality of modulators in separate cultures and determining the status of an activatable element in the cell population. In some embodiments, the status of a plurality of activatable elements in the cell population is determined. In some embodiments, at least one of the modulators of the plurality of modulators is an inhibitor. The modulator can be at least one of the modulators described herein. In some embodiments, at least one modulator is selected from the group consisting of SDF-1α, IFN-α IFN-γ, IL-10, IL-6, IL-27, G-CSF, FLT-3L, IGF-1, M-CSF, SCF, PMA, Thapsigargin, H₂O₂, etoposide, AraC, daunorubicin, staruosporine, and benzyloxycarbonyl-Val-Ala-Asp (OMe) fluoromethylketone (ZVAD), IL-3, IL-4, GM-CSF, EPO, LPS, TNF-α, and CD4OL, and a combination thereof.

In some embodiments of the invention, the status of an activatable element is determined by contacting the cell population with a binding element that is specific for an activation state of the activatable element. In some embodiments, the status of a plurality of activatable elements is determined by contacting the cell population with a plurality of binding elements, where each binding element is specific for an activation state of an activatable element.

In some embodiments, the methods of the invention provide methods for determining a phenotypic profile of a population of cells by exposing the population of cells to a plurality of modulators (recited herein) in separate cultures, wherein at least one of the modulators is an inhibitor, determining the presence or absence of an increase in activation level of an activatable element in the cell population from each of the separate cultures and classifying the cell population based on the presence or absence of the increase in the activation of the activatable element from each of the separate culture.

Patterns and profiles of one or more activatable elements are detected using the methods known in the art including those described herein. In some embodiments, patterns and profiles of activatable elements that are cellular components of a cellular pathway or a signaling pathway are detected using the methods described herein. For example, patterns and profiles of one or more phosphorylated polypeptides are detected using methods known in art including those described herein.

In some embodiments, the invention provides methods to carry out multiparameter flow cytometry for monitoring phospho-protein responses to various factors in myeloproliferative neoplasms at the single cell level. Phospho-protein members of signaling cascades and the kinases and phosphatases that interact with them are required to initiate and regulate proliferative signals in cells. Apart from the basal level of protein phosphorylation alone, the effect of potential drug molecules on these network pathways was studied to discern unique cancer network profiles, which correlate with the genetics and disease outcome. Single cell measurements of phospho-protein responses reveal shifts in the signaling potential of a phospho-protein network, enabling categorization of cell network phenotypes by multidimensional molecular profiles of signaling. The flow cytometry analysis may measure 1, 2, 3, 4, 5, 6, 7, 8, 9, 10 or more parameters in parallel. See U.S. Pat. No. 7,393,656. See also IRISH et. al., Single cell profiling of potentiated phospho-protein networks in cancer cells. Cell. 2004, vol. 118, p. 1-20. By way of example, flow cytometry can be used to measure at least 106 parameters for 32 or more primary samples.

Flow cytometry is useful in a clinical setting, since relatively small sample sizes, as few as 10,000 cells, can produce a considerable amount of statistically tractable multidimensional signaling data and reveal key cell subsets that are responsible for a phenotype U.S. Pat. Nos. 7,381,535 and 7,393,656. See also Krutzik et al., 2004).

Another embodiment of the present invention involves the ability to multiplex. As shown in FIGS. 10 and 11, multiple cell types may be contacted with multiple modulators (also called stims) in fewer wells or fluid volumes. For example, in one embodiment, three cell types, such as monocytes, T-cells, and B-cells, may be contacted with modulators that are specific to those cell types. Example modulators would be GM-CSF for monocytes, IL-2 for T-cells, and CD4OL for B-cells. However, different cell types and modulators may also be used. Then, the cells are contacted with various detection elements including but not limited to, fluorochrome-conjugated antibodies that recognize stretches of amino acids also called epitopes within cell surface and intracellular proteins such that the effect for any test compound, such as a drug, may be determined. In some embodiments of the present invention, 2, 3, 4, 5, 6, or more cell types may be present in one well will be analyzed. The internal or external markers may be separate and independent of each other or may have some interrelationship. One embodiment of the present invention allows for a more efficient use of cells, and reagents all of which provide internal controls that provide a high level of assay reproducibility. See the text below for more info on cell types, modulators, and detection elements.

### Disease Conditions

The methods of the invention are applicable to any condition in an individual involving, indicated by, and/or arising from, in whole or in part, altered physiological status in a cell. The term “physiological status” includes mechanical, physical, and biochemical functions in a cell. In some embodiments, the physiological status of a cell is determined by measuring characteristics of cellular components of a cellular pathway. Cellular pathways are well known in the art. In some embodiments the cellular pathway is a signaling pathway. Signaling pathways are also well known in the art (see, e.g., Hunter T., Cell 100(1): 113-27 (2000); Cell Signaling Technology, Inc., 2002 Catalogue, Pathway Diagrams pgs. 232-253). For examples of phospho-proteins and corresponding signaling pathways, see Table 6. A condition involving or characterized by altered physiological status may be readily identified, for example, by determining the state in a cell of one or more activatable elements, as taught herein.

In some embodiments, the present invention is directed to methods for analyzing the effects of a compound designed to inhibit Jak2s on one or more cells in a sample derived from an individual having or suspected of having a condition. For example, conditions include any solid of hematological malignancy or neoplasm, as well as MPN, AML, MDS. See U.S. Application No. 61/085,789 for further discussion on these diseases. Further examples include autoimmune, diabetes, cardiovascular, viral and other disease conditions. In some embodiments, the invention allows for identification of prognostically and therapeutically relevant subgroups of the conditions and prediction of the clinical course of an individual.

### Hematopoietic Disorders

Hematopoietic cells are blood-forming cells in the body. Hematopoiesis, or the development of blood cells, begins in the bone marrow. Depending on the cell type, further maturation occurs either in the periphery or in secondary lymphoid organs such as the spleen or lymph nodes. Hematopoietic disorders are recognized as clonal diseases, which are initiated by somatic and/or inherited mutations that cause dysregulated signaling in a progenitor cell. The wide range of possible mutations and accompanying signaling defects accounts for the diversity of disease phenotypes observed within this group of disorders. Hematopoietic disorders fall into three major categories: Myelodysplastic syndromes, myeloproliferative disorders or myeloproliferative neoplasms, and acute leukemias.

Myelodysplastic syndromes (MDSs) are characterized by a loss of mature blood cells in the periphery (anemia) due to hyperproliferation of progenitor cells with concomitant cell death in the bone marrow. This category of malignancies includes, but is not limited to, refractory anemia, refractory anemia with sideroblasts, refractory anemia with excess blasts, refractory anemia with excess blasts in transformation, refractory cytopenia with multilineage dysplasia, myelodysplastic syndrome with 5q-syndrome, and therapy-related myelodysplastic syndrome.

Myeloproliferative disorders (MPDs), now commonly referred to as meyloproliferative neoplasms (MPNs), are in the class of haematological malignancies that are clonal disorders of hematopoietic progenitors. Tefferi, A. and Vardiman, J. W., Classification and diagnosis of myeloproliferative neoplasms: The 2008 World Health Organization criteria and point-of-care diagnostic algorithms, Leukemia, September 2007, 22: 14-22, is hereby incorporated by reference. They are characterized by enhanced proliferation and survival of one or more mature myeloid lineage cell types. This category includes but is not limited to, chronic myeloid leukemia (CML), polycythemia vera (PV), essential thrombocythemia (ET), primary or idiopathic myelofibrosis (PMF), chronic neutrophilic leukemia, chronic eosinophilic leukemia, chronic myelomonocytic leukemia, juvenile myelomonocytic leukemia, hypereosinophilic syndrome, and systemic mastocytosis. Tefferi, A. and Gilliland, D. G., Oncogenes in myeloproliferative disorders, Cell Cycle. March 2007, 6(5): 550-566 is hereby fully incorporated by reference in its entirety for all purposes.

Acute leukemias are characterized by excessive proliferation of poorly differentiated myeloid or lymphoid cells. The WHO defines acute leukemia by the presence of 20% or more blasts in the blood or bone marrow. Acute leukemias are often preceded by MDS or MPN. Under the prevailing ‘two-hit’ model, MPN or MDS transforms to leukemia upon acquiring additional somatic mutations. Kelly, L. M. and Gilliland, D. G. Genetics of myeloid leukemias. Annu. Rev. Genomics. Hum. Genet., September 2002, 3: 179-198 is hereby fully incorporated by reference in its entirety for all purposes. This category includes, but is not limited to, acute myeloid leukemia, acute lymphoblastic leukemia, acute biphenotypic leukemia, precursor acute lymphoblastic leukemia, and aggressive NK cell leukemia. Golub et al., Molecular Classification of Cancer: Class Discovery and Class Prediction by Gene Expression Monitoring, Science, Oct. 15, 1999, 286: 531-537 is hereby fully incorporated by reference in its entirety for all purposes.

### Myeloproliferative Neoplasm (MPN)

MPNs are a group of disorders that cause an overproduction of blood cells (platelets, white blood cells and red blood cells) in the bone marrow. MPNs include polycythemia vera (PV), primary or essential thrombocythemia (ET), primary or idiopathic myelofibrosis, chronic myelogenous (myelocytic) leukemia (CML), chronic neutrophilic leukemia (CNL), and chronic eosinophilic leukemia (CEL)/hyper eosinophilic syndrome (HES). These disorders are grouped together because they share some or all of the following features: involvement of a multipotent hematopoietic progenitor cell, dominance of the transformed clone over the non-transformed hematopoietic progenitor cells, overproduction of one or more hematopoietic lineages in the absence of a definable stimulus, growth factor—independent colony formation in vitro, marrow hypercellularity, megakaryocyte hyperplasia and dysplasia, abnormalities predominantly involving chromosomes 1, 8, 9, 13, and 20, thrombotic and hemorrhagic diatheses, exuberant extramedullary hematopoiesis, and spontaneous transformation to acute leukemia or development of marrow fibrosis but at a low rate, as compared to the rate in chronic myelogenous leukemia (CML). The incidence of MPNs in the USA is 1.3 per 100,000 per year, with a maximum peak at the age of 25-60 years. (PCT/WO 2007085958 A2/3 (CONSORZIO PER GLI STUDI UNI IN) Feb. 8, 2007)

Many individuals with MPNs are asymptomatic at the time of diagnosis. A common sign for the presence of an MPN is enlarged spleen (except in the case of primary or essential thrombocythemia). Depending on the kind of disorder, symptoms may vary from person to person. Polycythemia vera (PV) is characterized by an increased production of blood cells, particularly red blood cells, by the bone marrow. This overproduction can lead to thickening of the blood, which can impair the functioning of the heart or the brain. Some symptoms specifically include fatigue, general malaise, difficulty in breathing, intense itching after bathing in warm water, stomach aches, purple spots or patches on the skin, nosebleeds, gum or stomach bleeding, blood in the urine, throbbing and burning pain in the skin often with darkened, blotchy areas, headache and visual disturbances, high blood pressure, and blockage of blood vessels. Blood clots may cause a heart disease, stroke, or gangrene (tissue death) of the extremities. MPNs predominantly occur in people older than 60 years, though 20 percent of cases occur in individuals of 40 years or less. Men are two times more likely to develop PV than women. Environmental factors, such as exposure to chemicals in hair dyes or to electrical wiring increase an individual's susceptibility to MPNs. Polycythemia vera has a survival rate of between 10 and 20 years, with the longest survival occurring in young age groups.

Primary or essential thromboycythemia is a result of overproduction of platelet cells. Symptoms include heart attack or stroke, headache, burning or throbbing pain, redness and swelling of hands and feet, bruising, gastrointestinal bleeding or blood in the urine. Similar to PV, it occurs primarily after 60 years of age, but some cases (20%) occur in persons under 40 years of age. Women are 1.5 times more likely to develop ET than men. Individuals with ET have normal life expectancy with only a low risk of developing cancer.

Primary or idiopathic myelofibrosis (also known as myelosclerosis) is caused by overproduction of collagen or fibrous tissue in the bone marrow. Other symptoms include fatigue, general malaise, difficulty breathing, weight loss, fever and night sweats, and abnormal bleeding. Individuals between the 60 and 70 years are most likely to develop the condition. Exposure to petrochemicals (such as benzene and toluene) and intense radiation may increase an individual's risk of developing the condition. Severe cases of primary myelofibrosis may be fatal within three to six years.

CML is a cancer of the bone marrow that produces abnormal granulocytes in the bone marrow. In the chronic phase of the disease, symptoms specific to CML include fatigue, general malaise, weight loss or loss of appetite, fever and night sweats, bone or joint pain, heart attack or stroke, difficulty in breathing, and gastrointestinal bleeding and infection. Individuals between 45 and 50 years are the most likely to develop the condition. Exposure to intense radiation may increase an individual's risk of developing the condition. Individuals with CML have a median survival rate of four to five years after diagnosis. The median survival rate is reduced to three months if CML transforms to acute leukemia.

Chronic neutrophilic leukemia is a rare entity characterized by persistent mature neutrophilia and hepatosplenomegaly, elevated serum B 12 levels, hyperuricemia, and raised alkaline phosphatase levels. It occurs at old age, i.e., around 62 years. The overall median survival is 30 months, with a five-year survival of 28 percent. Most patients have peripheral leukocytosis with a mean leukocyte count of 54×109 cells/L with predominant segmented and band cells.

Hypereosinophilic syndrome (HES) is characterized by an overproduction of eosinophils that cause organ damage. Hypereosinophilic syndrome is more common in men than in women (a ratio of nine to one) and occurs predominantly between 20 and 50 years of age. Clinical manifestations are a result of eosinophilic infiltration in tissues, release of eosinophilic products, and induction of a hypercoagulable state. Multiple organ systems are generally affected, including but not limited to, the central nervous system with peripheral neuropathies, hemiplegia, paraplegia encephalopathy, memory loss and ataxia. Some gastrointestinal manifestations are diarrhoea, hepatosplenomegaly, hepatic dysfunction, ascites, chronic active hepatitis and sclerosing cholangitis. Renal manifestations include acute renal failure, chronic renal failure, immunotactoid glomerulopathy, crescentic glomerulonephritis, and membranous glomerulopathy. Anemia, thrombocytopenia and thrombotic episodes are the common hematological manifestations. Skin manifestations are non-specific. Rashes can be macular, papulo vesicular or maculopapular. Urticaria and angioedema may be seen. HES is difficult to differentiate from eosinophilic leukemia, since both have common features at presentation. However, eosinophilic leukemia may be associated with clonality, abnormal karyotyping and presence of more than five percent blasts in the marrow and more than 25 percent immature eosinophils in peripheral smear or marrow. (VENKATESH C, et. al., Hypereosinophilic Syndrome. Departments of Pediatrics, Pediatric Gastroenterology and Pediatric Nephrology, Kanchi Kamakoti Childs Trust Hospital, Chennai.)

### Diagnosis

Elevated hematocrit or elevated platelet count suggests PV or ET. In PV, the frequencies of venous and arterial thrombosis are about equal, whereas venous thrombosis is less common in ET. PV is diagnosed when an increased hematocrit is accompanied by a Jak2 mutation. ET is diagnosed by exclusion.

Primary myelofibrosis is characterized by fibrotic bone marrow that cannot be explained by another diagnosis such as CML or MDS.

Among the MPNs, only CML can be reliably diagnosed by cytogenetics (the t(9;22) Philadelphia chromosome translocation is detected in 95 percent of the cases.) Fluorescent in situ hybridization or PCR can be used to confirm the presence of the BCR/ABL fusion gene.

Cytogenetics, in the diagnosis of chronic neutrophilic leukemia, shows abnormalities in 37 percent of the cases. Trisomy 8, trisomy 21 and deletions 20 are the most common observations.

One embodiment of the invention combines one or more of these existing tests with the analysis of signaling mediated by receptors to diagnose disease especially MDS, AML, or MPNs. All tests especially may be performed in one location and provided as a single service to physicians or other caregivers.

### Cell-Signaling Pathways and Differentiating Factors Involved

Dysregulation of the JAK/STAT signaling pathway has been implicated in the development and progression of MPNs. Activation of the JAK/STAT pathway results in phosphorylation and dimerization of Stat proteins which translocate to the nucleus, where they regulate a transcriptional program (Darnell et al., Science (1994)). Jak-2 is essential for signaling by receptors for many growth factors and cytokines, including but not limited to, growth hormone, prolactin, erythropoietin, thrombopoietin, interleukin-3, interleukin-5 (Yamaoka et al., Genome boil. (2004)). Dysregulation of Jak-2 has been implicated in several hematological malignancies by mechanisms, including but not limited to, acquired gain of function mutations such as V617F in the Jak2 JH2 domain. James et al., Nature (2005) 434: p. 1144, Levine et al., Cancer Cell, (2005) 7:p. 387, Kralvics et al., New England J. Med. (2005) 352: p. 1779, Baxter et al., Lancet (2005) 365: p 1779 are hereby fully incorporated by reference in its entirety for all purposes. Several distinct MPNs, such as PV, ET, and myelofibrosis, are found to have the Jak2-V617F mutation, supporting the concept that hyperactivation of JAK/STAT singaling is involved in the development of MPNs. Jak2 mutations are present in virtually all cases of PV, 41 to 72 percent of ET cases, and 39 to 57 percent of primary myelofibrosis cases. Baxter et al., Acquired mutation of the tyrosine kinase Jak2 in human myeloproliferative disorders. Lancet. Mar. 19-25, 2005, 365(9464): 1054-1061 is hereby fully incorporated by reference in its entirety for all purposes. Studies have found 15 gene-expression markers that were elevated in patients with PV, including polycythemiarubra vera 1 (PRV1) and nuclear factor erythroid-derived 2 (NF-E2), as well as one marker that was down regulated, ANKRD15. (Kralovics et al., Altered gene expression in myeloproliferative disorders correlates with the activation of singaling by the V617F mutation of Jak2, Blood. November 2005, 106(10): 3374-76).

In CML, the BCR/ABL fusion gene product of the Philadelphia chromosome exhibits persistent tyrosine kinase activity and Stat5 phosphorylation. (H. Yu and R. Jove, The STATs of cancer? New molecular targets come of age, Nat. Rev. Cancer, Feb. 1, 2004, 4: 97-105, is hereby fully incorporated by reference in its entirety for all purposes. Similarly, a fusion gene product of FIP1L/PDGFRA is implicated in a subset of hypereosinophilic syndrome patients with an interstitial deletion in chromosome 4q12. Both of these fusion gene products are exquisitely sensitive to inhibition by the targeted kinase inhibitor, imatinib (Gleevec). (Crescenzi et al., FIP1L1-PDGFRA in chronic eosinophilic leukemia and BCR-ABL1 in chronic myeloid leukemia affect different leukemic cells, Leukemia, 2007, 21(3): 397-402).

In some embodiments, the methods of the invention are employed to determine the status of an activatable element in a signaling pathway. In some embodiments, a cell is classified, as described herein, according to the activation level of one or more activatable elements in one or more signaling pathways. Signaling pathways and their members have been described. See (Hunter T. Cell Jan. 7, 2000; 100(1): 13-27). Exemplary signaling pathways downstream of Jak-2 include the following pathways and their members: The MAP kinase (MAPK) pathway including Ras, Raf, MEK, ERK and elk; the PI3K/Akt pathway including PI-3-kinase, PDK1, Akt and Bad; the NF-kB pathway including IKKs, IkB and the Wnt pathway including frizzled receptors, beta-catenin, APC and other co-factors and TCF (see Cell Signaling Technology, Inc. 2002 Catolog pages 231-279 and Hunter T., supra.). In some embodiments of the invention, the correlated activatable elements being assayed (or the signaling proteins being examined) are members of the MAP kinase, Akt, NFkB, WNT, RAS/RAF/MEK/ERK, JNK/SAPK, p38 MAPK, Src Family Kinases, JAK/STAT and/or PKC signaling pathways. For an in-depth discussion of these signaling pathways, please refer to U.S. Patent Application No. 61/265,743, which is hereby fully incorporated by reference in its entirety.

One embodiment of the invention will look at any of the cell signaling pathways described above in classifying diseases, such as MPNs. Modulators or inhibitors can be designed to investigate these pathways and any relevant parallel pathways.

### Therapeutic Agents Effective Against the Disease

There is strong evidence for the efficacy of targeted kinase inhibitors in certain MPNs, and the success of these drugs has triggered rampant development of additional therapies in this class. However, until new targeted drugs become available, most of the MPNs must still be managed with traditional therapies. Depending on the type and severity of the disorder, various treatments are available that help improve symptoms and prevent further complexities.

For the treatment of polycythemia vera, phlebotomy, or the removal of one unit of blood, is performed on a regular basis. This prevents accumulation of blood and reduces the risk of stroke. Chemotherapy is preferred to control excess production of red blood cells if the patient has experienced blood clotting. Interferon can also be used to treat this disease.

Essential thrombocythemia can be treated with drugs that slow down platelet production and possibly with chemotherapy. Various medications may be used to reduce platelets, including hydroxyurea, anagrelide, interferon, and busulfan. Each medication has its own side effects, and treatment needs to be tailored to each patient. Aspirin may be appropriate for many ET patients to prevent of blood clots and to treat other ET related symptoms. However, in patients with very high platelet counts, aspirin may lead to bleeding.

Treatment of myelofibrosis generally involves blood cell transfusion to increase the number of red blood cells. Interferon can slow the progression of this disease and some patients benefit from splenectomy. In some cases, bone marrow transplantation is also performed.

Imatinib or the related molecule dasatinib are now used as the primary treatment of chronic myeloid leukemia. These molecules block the tyrosine kinase activity of BCR/ABL proteins, present in nearly all CML patients, essentially stopping the production of excess white blood cells. Treatment of CML with imatinib is extremely successful, leading to complete remission in 97% of patients treated at the early stages of the disease. Kantarjian et al., Imatinib mesylate therapy in newly diagnosed patients with Philadelphia chromosome-positive chronic myelogenous leukemia: high incidence of early complete and major cytogenetic responses, Blood, 2003, 101(1): 97-100 is hereby fully incorporated by reference in its entirety for all purposes. Dasatinib, which is more potent than imatinib, induced major hematologic response in 34% of advanced stage (blast crisis) CML patients. Cortes et al., Dasatinib induces complete hematologic and cytogenetic responses in patients with imatinib-resistant or -intolerant chronic myeloid leukemia in blast crisis, Blood, 2007, 109(8): 3207-13 is hereby fully incorporated by reference in its entirety for all purposes.

In younger individuals allogeneic bone marrow transplantation represents a potentially curative treatment modality in the management of chronic neutrophilic leukemia. Oral chemotherapy including hydroxyurea and busulphan has been used to control hyperleukocytosis. Alpha interferon therapy similar to CML has also been tried.

Hypereosinophilic syndrome symptoms are treated with drugs, such as imatinib, infliximab, glucocorticoids, hydroxyureas, cyclosporin and interferon alpha. Cardiac or neurological dysfunction at the onset results in aggressive clinical course and treatment failure. A subset of patients are sensitive to imatinib mesylate. Other therapies include monoclonal anti-IL5 antibody and stem cell transplantation.

These hematopoietic disorders can be better classified by using multiparametric phospho-protein analysis because this invention would involve a biologically based classification system. For example, the present invention could: enable patient stratification which would provide an improved classification of these diseases; be used for drug screening to produce biologically informed therapeutics choices; and address the potential for responsiveness to new therapies. The benefits of using the present invention for diagnostic tests includes defining the therapeutic possibilities; identification of aggressive disease giving potentially improved outcomes; and matching signaling profiles to experimental therapeutic outcomes. Additionally, elucidation of disease mechanisms would identify de novo targets applicable to future drug therapy and cohort selection for drug development.

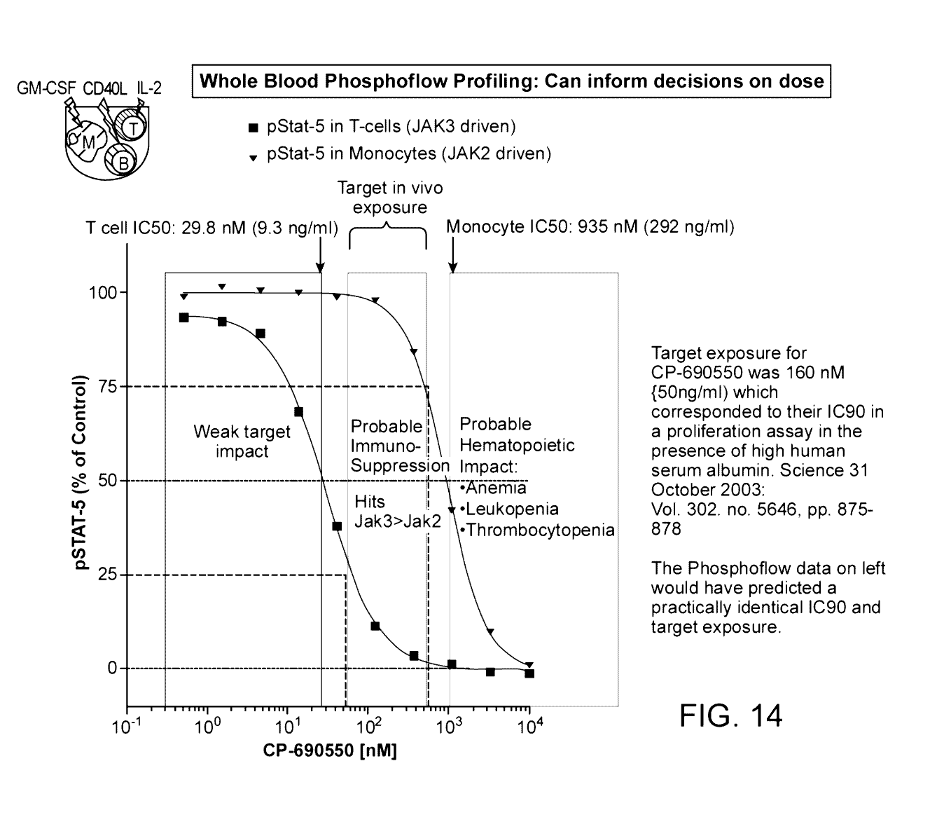

One embodiment of the invention involves analyzing cell singaling pathways mediated by receptors and thereafter administering the above therapeutic agents in response to a diagnosis. Future therapeutic agents may also be prescribed based on this analysis. The methods of the invention may also be used to compare patient response to therapeutics over time, to identify, for example the development of drug resistance. For example, in FIG. 14, multiparameter phosphoflow is used to analyze JAK/STAT signaling at time of diagnosis, and again at time of relapse.

### Compounds to be Analyzed

The methods and compositions of the invention may be employed for screening compounds such as inhibitors against biological targets including but not limited to kinase inhibitors, transcription factor inhibitors, histone deacetylase inhibitors, DNA-Methyl transferase inhibitors and other compounds in a way that can simultaneously distinguish different cell types and measure the effects of a compound on several different cellular pathways in each cell type as well as upstream or downstream effects. In one embodiment, compounds are tested for selectivity simultaneously or sequentially across one or more cellular pathways and one or more cell types. In another embodiment, compounds are tested for potency across one or more cellular pathways and one or more cell types simultaneously or sequentially. Additionally, in some embodiments, compounds may be tested for both potency and selectivity.

Compounds that are analyzed in some embodiments of the present invention are designed to treat cancer. The compounds can comprise a binding element and an active component designed to induce cell death or apotosis. In some embodiments, the binding component is directed at a cell surface antigen, whereby the compound may be internalized and cleaved into the binding component and the active component. Active components may be cytotoxic agents or cancer chemotherapeutic agents. Binding agents can be antibodies, antibody fragments, such as single chain fragments, binding peptides, or any compound that can bind a specific cellular element to facilitate entry into the cell to carry the compound that acts on the cell. See Ricart, A D, and Tolcher, A W, Nat Clin Pract Oncol, 2007 April; 4(4):245-55; Singh et al., Curr Med Chem. 2008; 15(18):1802-26.

Active compounds that can be delivered to the cell using a binding component include agents that induce cell death or apoptosis. These agents may be common cytotoxic agents that are used in cancer chemotherapy, or any other agents that are just generally toxic to cells. Example agents include targeted therapies, such as small molecules directed to biological targets.

Some compounds that contain binding elements attached to elements that can kill or render cells apoptotic are called antibody-drug conjugates. Antibodies are chosen for their ability to selectively target cells with receptors common to tumors. See DiJoseph F, Goad M E, Dougher M M, et al. Potent and specific antitumour efficacy of CMC-544, a CD22-targeted immunoconjugate of calicheamicin, against systemically disseminated B cell lymphoma. Clin Cancer Res. 2004; 10:8620-8629. Upon binding of the antibody—drug conjugate (ADC) to cells, the ADC-receptor complex is internalized into the cell, where the cytotoxic drug is released. Cytotoxic drugs are therefore selected for their potential to induce cell death from within the tumor cell. The molecules that link the antibody to the cytotoxic agent are chosen for their ability to stabilize the conjugate and thus minimize release of the drug before the ADC is internalized into the tumor cell. See Hamann P R. Monoclonal antibody—drug conjugates. Expert Opin Ther Patents. 2005; 15:1087-1103; Mandler R, Kobayashi H, Hinson E R, et al. Herceptin-geldanamycin immunoconjugates: pharmacokinetics, biodistribution, and enhanced antitumor activity. Cancer Res. 2004; 64:1460-1467; and Sanderson R J, Hering M A, James S F, et al. In vivo drug-linker stability of an anti-CD30 dipeptide-linked auristatin immunoconjugate. Clin Cancer Res. 2005; 11:843-852.

In some embodiments, compounds are small-molecule inhibitors of JAK/STAT signaling. Many small-molecule inhibitors of Jak2 and other kinases are actively being developed by various pharmaceutical companies. Examples of Jak2 inhibitors and other compounds currently in development, including but not limited to: AZ-01, AZ-60, AZD 1480 (AstraZeneca—Jak2 inhibitor); ON-044580 (Onconova—non-ATP-competitive Jak2 inhibitor); SGI-1252 (SuperGen—orally available Jak2 inhibitor); TG-101348/TG-101193/TG-101209 (TargeGen—dual Jak2/Flt3 inhibitors); ITF2357 (Italfarmaco); INCB-18424, INCB-28050 (Incyte); CP-690,550; CEP-701 (Cephalon); MK-0683 (Copenhagen University Hospital Herlev-HDAC inhibitor); SB-1518, SB-1578/ONX-0805 (S*Bio); XL019 (Exelixis); bevacizumab/Avastin (Myeloproliferative DRC); Dasatinib (Bristol-Myers Squibb); Cyt-387 (Cytopia-Jak2 inhibitor); WP-1066, WP-1130 (MD Anderson Cancer Center); and VX-509 (Vertex Pharmaceuticals).

In some embodiments, the JAK/STAT inhibitor compounds act by selectively inhibiting Jak2 through the tyrphostin scaffold, tyrosine phosphorylation inhibitor. Whereas in some embodiments, the Jak2 inhibitor compounds are non-selective inhibitors of Jak2.

### (a) Activatable Elements

The methods and compositions of the invention may be employed to examine and profile the status of any activatable element alone or in combination with other activatable elements in a cellular pathway. Single or multiple distinct pathways may be profiled sequentially or simultaneously, or subsets of activatable elements within a single pathway or across multiple pathways may be examined sequentially or simultaneously. In one embodiment, the cell is a hematopoietic cell. Examples of hematopoietic cells include, but are not limited to pluripotent hematopoietic stem cells, granulocyte lineage progenitor and/or derived cells, monocyte lineage progenitor and/or derived cells, macrophage lineage progenitor and derived cells, megakaryocyte lineage progenitor and/or derived cells and erythroid lineage progenitor and/or derived cells, lymphoid progenitors and/or derived cells.

As will be appreciated by those in the art, a wide variety of activation events may be used in the present invention. In a preferred embodiment two or more activation states are differentiated using detectable events or moieties. Activation results in a change in the activatable element that is detectable by an activation state indicator, preferably by altered binding of a labeled binding element or by changes in detectable biological activities. For example, the change in activation state of an activatable element may be measured by phophorylation of an amino acid such as tyrosine, serine or threonine. A second example, the activated state has an enzymatic activity which can be measured and compared to a lack of activity in the non-activated state.

As an illustrative example, and without intending to be limited to any theory, an individual phosphorylatable site on a protein can activate or deactivate the protein. Additionally, phosphorylation of an adapter protein may promote its interaction with other components/proteins of distinct cellular signaling pathways. The terms “on” and “off,” when applied to an activatable element that is a part of a cellular constituent, are used here to describe the state of the activatable element, and not the overall state of the cellular constituent of which it is a part. Typically, a cell possesses a plurality of a particular protein or other constituent with a particular activatable element and this plurality of proteins or constituents usually has some proteins or constituents whose individual activatable element is in the on state and other proteins or constituents whose individual activatable element is in the off state. Since the activation state of each activatable element is measured through the use of a binding element that recognizes a specific activation state, only those activatable elements in the specific activation state recognized by the binding element, representing some fraction of the total number of activatable elements, will be bound by the binding element to generate a measurable signal. The measurable signal corresponding to the summation of individual activatable elements of a particular type that are activated in a single cell is the “activation level” for that activatable element in that cell.

Activation levels for a particular activatable element may vary among individual cells so that when a plurality of cells is analyzed, the activation levels follow a distribution. The distribution may be a normal distribution, also known as a Gaussian distribution, or it may be of another type. Different populations of cells may have different distributions of activation levels that can then serve to distinguish between the populations.

In some embodiments, the basis for classifying cells is that the distribution of activation levels for one or more specific activatable elements will differ among different phenotypes. A certain activation level, or more typically a range of activation levels for one or more activatable elements seen in a cell or a population of cells, is indicative that that cell or population of cells belongs to a distinctive phenotype. Other measurements, such as cellular levels (e.g., expression levels) of biomolecules that may not contain activatable elements, may also be used to classify cells in addition to activation levels of activatable elements; it will be appreciated that these levels also will follow a distribution, similar to activatable elements. Thus, the activation level or levels of one or more activatable elements, optionally in conjunction with levels of one or more levels of biomolecules that may or may not contain activatable elements, of cell or a population of cells may be used to classify a cell or a population of cells into a class. Once the activation level of intracellular activatable elements of individual single cells is known they can be placed into one or more classes, e.g., a class that corresponds to a phenotype. A class encompasses a class of cells wherein every cell has the same or substantially the same known activation level, or range of activation levels, of one or more intracellular activatable elements. For example, if the activation levels of five intracellular activatable elements are analyzed, predefined classes of cells that encompass one or more of the intracellular activatable elements can be constructed based on the activation level, or ranges of the activation levels, of each of these five elements. It is understood that activation levels can exist as a distribution and that an activation level of a particular element used to classify a cell may be a particular point on the distribution but more typically may be a portion of the distribution.

In some embodiments, the physiological status of one or more cells is determined by examining and profiling the activation level of one or more activatable elements in a cellular pathway. In some embodiments, a cell is classified according to the activation level of a plurality of activatable elements. In some embodiments, a hematopoietic cell is classified according to the activation levels of a plurality of activatable elements. In some embodiments, 1, 2, 3, 4, 5, 6, 7, 8, 9, 10 or more activatable elements may be analyzed in a cell signaling pathway. In some embodiments, the activation levels of one or more activatable elements of a hematopoietic cell are correlated with a condition. In some embodiments, the activation levels of one or more activatable elements of a hematopoietic cell are correlated with a neoplastic or hematopoietic condition as described herein. Examples of hematopoietic cells include, but are not limited to, AML, MDS or MPN cells.

In some embodiments, the activation level of one or more activatable elements in single cells in the sample is determined. Cellular constituents that may include activatable elements include without limitation proteins, carbohydrates, lipids, nucleic acids and metabolites. The activatable element may be a portion of the cellular constituent, for example, an amino acid residue in a protein that may undergo phosphorylation, or it may be the cellular constituent itself, for example, a protein that is activated by translocation, change in conformation (due to, e.g., change in pH or ion concentration), by proteolytic cleavage, degradation through ubiquitination and the like. Upon activation, a change occurs to the activatable element, such as covalent modification of the activatable element (e.g., binding of a molecule or group to the activatable element, such as phosphorylation) or a conformational change. Such changes generally contribute to changes in particular biological, biochemical, or physical properties of the cellular constituent that contains the activatable element. The state of the cellular constituent that contains the activatable element is determined to some degree, though not necessarily completely, by the state of a particular activatable element of the cellular constituent. For example, a protein may have multiple activatable elements, and the particular activation states of these elements may overall determine the activation state of the protein; the state of a single activatable element is not necessarily determinative. Additional factors, such as the binding of other proteins, pH, ion concentration, interaction with other cellular constituents, and the like, can also affect the state of the cellular constituent.

In some embodiments, the activation levels of a plurality of intracellular activatable elements in single cells are determined. Activation states of activatable elements may result from chemical additions or modifications of biomolecules and include many biochemical processes. See U.S. Application No. 61/265,743, which is incorporated by reference.

In some embodiments, cellular redox signaling nodes are analyzed for a change in activation level. Reactive oxygen species (ROS) are involved in a variety of different cellular processes ranging from apoptosis and necrosis to cell proliferation and carcinogenesis. ROS can modify many intracellular signaling pathways including protein phosphatases, protein kinases, and transcription factors. This activity may indicate that the majority of the effects of ROS are through their actions on signaling pathways rather than via non-specific damage of macromolecules. The exact mechanisms by which redox status induces cells to proliferate or to die, and how oxidative stress can lead to processes evoking tumor formation are still under investigation. See Mates, J M et al., Arch Toxicol. 2008 May:82(5):271-2; Galaris D., et al., Cancer Lett. 2008 Jul. 18; 266(1)21-9.

Under normal physiological conditions, a balance exists between oxidants and anti-oxidants in a redox homeostasis. Severe disturbance of this homeostasis causes the accumulation of high levels of reactive oxygen species (ROS). ROS are derived from the reduction of molecular oxygen to generate superoxide which then is converted to other ROS species. ROS are produced primarily by three sources within the cell. The first and a major site of ROS generation is the mitochondrial electron transport chain where electrons escaping from their transport complexes react with oxygen to form superoxide. A second major source of ROS production are from the NADPH oxidase (Nox) complexes, which were originally identified in phagocytes as a key component of the human innate host defense. Subsequently Nox complexes were found in a wide variety of non-phagocytic cells and tissues and contribute to signal transduction, cell proliferation and apoptosis with roles in many physiological processes. Nox consists of membrane-bound subunits that need to interact with cytoplasmic regulatory subunits including the small GTPase Rac in order to become active and produce ROS (Ushio-Fukai and Nakamura, Cancer Lett. (2008) 266 p 37). There exists a family of Nox proteins and some of the family members are increased in cancer. The third source of ROS production is generated from other enzymes including xanthine oxidase, cyclooxygenases, lipoxygenases, myeloperoxidase, heme oxidase and cytochrome P450-based enzymes (Kuo., Antioxidants and Redox signaling (2009) 11 p 1). Cytokine growth factor and death receptor signaling can also lead to the production of ROS that function as second messengers playing an important role in signal transduction pathways. For example generation of peroxide transiently inhibits phosphatase activity in a variety kinase cascades (Morgan et al., Cell Research (2008) 18 p 343, Bindoli et al., Antioxidants and Redox Signaling (2008) 10 p 1549.).

As mentioned above, ROS can act as second messengers at submicromolar concentrations and when endogenously elevated they are reduced by anti-oxidants generated by enzymes, such as superoxide dismutase, glutathione peroxidase, catalase, thioredoxin reductase and glutathione S-transferase. Although these anti-oxidant enzymatic systems are considered the most specific and efficient modulators of cellular redox state, several other low molecular weight anti-oxidant states also exist. In particular the tripeptide, 7-glutamylcysteinylglycine (glutathione) exists at milli-molar concentrations inside the cell and is capable of reducing peroxide, lipid peroxides as well as protein disulfide bonds. By acting as an electron donor, glutathione itself gets oxidized to GSSH, and becomes the substrate for glutathione reductase that maintains it in its reduced form GSH. The ratio of reduced to oxidized glutathione is a measure of ROS in the cell. Further, glutathione reductase is constitutively active and induced upon oxidative stress.

In cancer, the intracellular redox potential can have a profound effect on the efficacy of therapeutic agents either through modulating drug transporter function or through changing the oxidation state and therefore activity of the therapeutic agent itself or through modulating drug transporter function such that agents will be extruded from the cell (Kuo, Antioxidants and Redox signaling (2009) 11 p 1, Karihatala et al., (2007) APMIS 115 p 81). As an example, Mylotarg, also called Gemtuzumab ozogamicin, consists of a humanized CD33 antibody conjugated to a DNA damaging agent, N-acetyl calicheamicin 1,2 dimethyl hydrazine dichloride. Once internalized the calicheamicin is released from the CD33 antibody through acid hydrolysis and in order for it to be active it needs to be reduced by glutathione. Thus, measuring the intracellular redox state could allow a prediction to be made of how cells will respond to Mylotarg. Another example in which the intracellular redox state plays a role in drug efficacy is for treatment of acute promyelocytic leukemia with arsenic trioxide. The proposed mechanism of action is an increase in NADPH oxidase-generated superoxide levels which promote apoptosis (Chou and Dang, Curr. Opin. Hem. (2004) 12 p 1).

Reactive oxygen species can be measured. One example technique is by flow cytometry. See Chang et al., Lymphocyte proliferation modulated by glutamine: involved in the endogenous redox reaction; Clin Exp Immunol. 1999 September; 117(3): 482-488. Redox potential can be evaluated by means of an ROS indicator, one example being 2′,7′-dichlorofluorescein-diacetate (DCFH-DA) which is added to the cells at an exemplary time and temperature, such as 37° C. for 15 minutes. DCF peroxidation can be measured using flow cytometry. See Yang K D, Shaio M F. Hydroxyl radicals as an early signal involved in phorbol ester-induced monocyte differentiation of HL60 cells. Biochem Biophys Res Commun. 1994; 200:1650-7 and Wang J F, Jerrells T R, Spitzer J J. Decreased production of reactive oxygen intermediates is an early event during in vitro apoptosis of rat thymocytes. Free Radic Biol Med. 1996; 20:533-42.

Other exemplary fluorescent dyes, include but are not limited to 2-(6-(4′-hydroxy)phenoxy-3H-xanthen-3-on-9-yl)benzoic acid (HPF) and 2-(6-(4′-amino)phenoxy-3H-xanthen-3-on-9-yl)benzoic acid (APF) which both detect ROS species (Setsukinai et al., J. Biol. Chem. (2003) 278 p 3170). Other fluorescent probes are derivatives of reduced fluorescein and calcein which are cell-permeant indicators for ROS. Chemically reduced and acetylated forms of, 2′,7′ dichlorofluorescein (DCF) and calcein are non-fluorescent until their acetate groups are removed by intracellular esterases (Molecular probes). Oxidation of what is now a charged form of the dye is mediated by intracellular ROS. This causes the dye to become fluorescent and the amount of fluorescence will be directly related to the intracellular ROS concentration. As an alternative to monitoring ROS levels, since glutathione levels profoundly influence the redox status, the use of ThiolTracker™ Violet can be used to its monitor levels (Molecular Probes).

In some embodiments, other characteristics that affect the status of a cellular constituent may also be used to classify a cell. Examples include the translocation of biomolecules or changes in their turnover rates and the formation and disassociation of complexes of biomolecule. Such complexes can include multi-protein complexes, multi-lipid complexes, homo- or hetero-dimers or oligomers, and combinations thereof. Other characteristics include proteolytic cleavage, e.g. from exposure of a cell to an extracellular protease or from the intracellular proteolytic cleavage of a biomolecule.

Additional elements may also be used to classify a cell, such as the expression level of extracellular or intracellular markers, nuclear antigens, enzymatic activity, protein expression and localization, cell cycle analysis, chromosomal analysis, cell volume, and morphological characteristics like granularity and size of nucleus or other distinguishing characteristics. For example, B cells can be further subdivided based on the expression of cell surface markers such as CD19, CD20, CD22 or CD23.

Alternatively, predefined classes of cells can be aggregated or grouped based upon shared characteristics that may include inclusion in one or more additional predefined class or the presence of extracellular or intracellular markers, similar gene expression profile, nuclear antigens, enzymatic activity, protein expression and localization, cell cycle analysis, chromosomal analysis, cell volume, and morphological characteristics like granularity and size of nucleus or other distinguishing cellular characteristics.

In one embodiment, the activatable enzyme is a caspase. The caspases are an important class of proteases that mediate programmed cell death (referred to in the art as “apoptosis”). Caspases are constitutively present in most cells, residing in the cytosol as a single chain proenzyme. These are activated to fully functional proteases by a first proteolytic cleavage to divide the chain into large and small caspase subunits and a second cleavage to remove the N-terminal domain. The subunits assemble into a tetramer with two active sites (Green, Cell 94:695-698, 1998). Many other proteolytically activated enzymes, known in the art as “zymogens,” also find use in the instant invention as activatable elements.

In an alternative embodiment the activation of the activatable element involves prenylation of the element. By “prenylation”, and grammatical equivalents used herein, is meant the addition of any lipid group to the element. Common examples of prenylation include the addition of farnesyl groups, geranylgeranyl groups, myristoylation and palmitoylation. In general these groups are attached via thioether linkages to the activatable element, although other attachments may be used.

In alternative embodiment, activation of the activatable element is detected as intermolecular clustering of the activatable element. By “clustering” or “multimerization”, and grammatical equivalents used herein, is meant any reversible or irreversible association of one or more signal transduction elements. Clusters can be made up of 2, 3, 4, etc., elements. Clusters of two elements are termed dimers. Clusters of 3 or more elements are generally termed oligomers, with individual numbers of clusters having their own designation; for example, a cluster of 3 elements is a trimer, a cluster of 4 elements is a tetramer, etc.

Clusters can be made up of identical elements or different elements. Clusters of identical elements are termed “homo” dimers, while clusters of different elements are termed “hetero” clusters. Accordingly, a cluster can be a homodimer, as is the case for the β2-adrenergic receptor.

Alternatively, a cluster can be a heterodimer, as is the case for GA_{B-R}. In other embodiments, the cluster is a homotrimer, as in the case of TNFα, or a heterotrimer such the one formed by membrane-bound and soluble CD95 to modulate apoptosis. In further embodiments the cluster is a homo-oligomer, as in the case of Thyrotropin releasing hormone receptor, or a hetero-oligomer, as in the case of TGFβ1.

In a preferred embodiment, the activation or signaling potential of elements is mediated by clustering, irrespective of the actual mechanism by which the element's clustering is induced. For example, elements can be activated to cluster a) as membrane bound receptors by binding to ligands (ligands including both naturally occurring or synthetic ligands), b) as membrane bound receptors by binding to other surface molecules, or c) as intracellular (non-membrane bound) receptors binding to ligands.

In a preferred embodiment the activatable elements are membrane bound receptor elements that cluster upon ligand binding such as cell surface receptors. As used herein, “cell surface receptor” refers to molecules that occur on the surface of cells, interact with the extracellular environment, and transmit or transduce (through signals) the information regarding the environment intracellularly in a manner that may modulate cellular activity directly or indirectly, e.g., via intracellular second messenger activities or transcription of specific promoters, resulting in transcription of specific genes. One class of receptor elements includes membrane bound proteins, or complexes of proteins, which are activated to cluster upon ligand binding. As is known in the art, these receptor elements can have a variety of forms, but in general they comprise at least three domains. First, these receptors have a ligand-binding domain, which can be oriented either extracellularly or intracellularly, usually the former. Second, these receptors have a membrane-binding domain (usually a transmembrane domain), which can take the form of a seven pass transmembrane domain (discussed below in connection with G-protein-coupled receptors) or a lipid modification, such as myristylation, to one of the receptor's amino acids which allows for membrane association when the lipid inserts itself into the lipid bilayer. Finally, the receptor has an signaling domain, which is responsible for propagating the downstream effects of the receptor.

Examples of such receptor elements include hormone receptors, steroid receptors, cytokine receptors, such as IL1-α, IL-β, IL-2, IL-3, IL-4, IL-5, IL-6, IL-7, IL-8, IL-9, IL-10. IL-12, IL-15, IL-18, IL-21, CCR5, CCR7, CCR-1-10, CCL20, chemokine receptors, such as CXCR4, adhesion receptors and growth factor receptors, including, but not limited to, PDGF-R (platelet derived growth factor receptor), EGF-R (epidermal growth factor receptor), VEGF-R (vascular endothelial growth factor), uPAR (urokinase plasminogen activator receptor), ACHR (acetylcholine receptor), IgE-R (immunoglobulin E receptor), estrogen receptor, thyroid hormone receptor, integrin receptors (β1, β2, β3, β4, β5, β6, α1, α2, α3, α4, α5, α6), MAC-1 (β2 and cd11b), αVβ33, opioid receptors (mu and kappa), FC receptors, serotonin receptors (5-HT, 5-HT6, 5-HT7), β-adrenergic receptors, insulin receptor, leptin receptor, TNF receptor (tissue-necrosis factor), statin receptors, FAS receptor, BAFF receptor, FLT3 LIGAND receptor, GMCSF receptor, and fibronectin receptor.

In a preferred embodiment, the activatable element is a Janus Kinase. The Janus kinases (Jaks) are a family of cytoplasmic non-receptor tyrosine kinases that mediate signals from receptors for cytokines, growth factors, and G-protein coupled receptors. There are four Jak kinases: Jak1, Jak2, Jak3, and TYK2 each with seven Jak homology (JH) domains. The C-terminal JH1 domain is the kinase domain while JH2 is a pseudokinase domain with a critical role in regulating the kinase activity of JH1.

Mutations in Jak proteins have been described for several myeloid malignancies. To date, the most prevalent mutation, found in MPNs, is V617F in the JH2 domain which disrupts the inhibitory role that JH2 has on JH1 thereby activating both the kinase and transforming activities of Jak2. This gain of function mutation is expressed in 81-99% PV, 41-72% ET and 39-57% PMF and with lesser prevalence in other leukemias. The small percentage of PV patients that are negative for the Jak2(V617F) mutation have somatic mutations within exon 12 (also JH2) of Jak2. A significant portion of ET and PMF patients are Jak2(V617F) negative and further sequencing studies of Jaks and STATs did not identify any additional mutations. However, given that in order to signal, Jak-2(V617F) must interact with a cytokine receptor, sequencing studies were undertaken to identify mutations in the receptors known to bind and activate Jak-2 that could confer activation of Jak-2 independently of a mutation within Jak-2 itself. In these studies, somatic mutations were identified in the transmembrane-juxtamembrane junction of the receptor for thrombopoietin called myeloproliferative leukemia virus proto-oncogene (MPLW515L/K/S, MPLS505N). Additionally, gain of function Jak-2 mutations resulting from chromosomal translocation have been associated with other myeloid leukemias and also in lymphoid leukemias.

The somatic mutations identified in Jak2 confer these proteins with properties that mediate factor-independent proliferation and transformation. However, the cytokine receptors must be present in order to provide a scaffold for Jak-2 allowing it to undergo transphosphorylation and activation. Downstream signaling from Jak2(V617F) and Jak2(exon 12) mutations results in the activation of signaling pathways, including but not limited to, signal transducers and activators of transcription (Stats), phosphatidylinositol 3′-kinase(PI3K)-Akt and mitogen activated protein kinases (MAPKs) such as Erk, p38 and JNK.

In one embodiment, the activatable element is a receptor tyrosine kinase. The receptor tyrosine kinases can be divided into subgroups on the basis of structural similarities in their extracellular domains and the organization of the tyrosine kinase catalytic region in their cytoplasmic domains. Sub-groups I (epidermal growth factor (EGF) receptor-like), II (insulin receptor-like) and the EPH/ECK family contain cysteine-rich sequences (Hirai et al., (1987) Science 238:1717-1720 and Lindberg and Hunter, (1990) Mol. Cell. Biol. 10:6316-6324). The functional domains of the kinase region of these three classes of receptor tyrosine kinases are encoded as a contiguous sequence (Hanks et al. (1988) Science 241:42-52). Subgroups III (platelet-derived growth factor (PDGF) receptor-like) and IV (the fibro-blast growth factor (FGF) receptors) are characterized as having immunoglobulin (Ig)-like folds in their extracellular domains, as well as having their kinase domains divided in two parts by a variable stretch of unrelated amino acids (Yanden and Ullrich (1988) supra and Hanks et al. (1988) supra). For further discussion, see U.S. Patent Application 61/120,320.

In another embodiment the receptor element is a member of the hematopoietin receptor superfamily. Hematopoietin receptor superfamily is used herein to define single-pass transmembrane receptors, with a three-domain architecture: an extracellular domain that binds the activating ligand, a short transmembrane segment, and a domain residing in the cytoplasm. The extracellular domains of these receptors have low but significant homology within their extracellular ligand-binding domain comprising about 200-210 amino acids. The homologous region is characterized by four cysteine residues located in the N-terminal half of the region, and a Trp-Ser-X-Trp-Ser (WSXWS) motif located just outside the membrane-spanning domain. Further structural and functional details of these receptors are provided by Cosman, D. et al., (1990). The receptors of IL-1, IL-2, IL-3, IL-4, IL-5, IL-6, IL-7, prolactin, placental lactogen, growth hormone GM-CSF, G-CSF, M-CSF and erythropoietin have, for example, been identified as members of this receptor family.

In a further embodiment, the receptor element is an integrin other than Leukocyte Function Antigen-1 (LFA-1). Members of the integrin family of receptors function as heterodimers, composed of various α and β subunits, and mediate interactions between a cell's cytoskeleton and the extracellular matrix. (Reviewed in, Giancotti and Ruoslahti, Science 285, 13 Aug. 1999). Different combinations of the α and β subunits give rise to a wide range of ligand specificities, which may be increased further by the presence of cell-type-specific factors. Integrin clustering is known to activate a number of intracellular signals, such as RAS, MAP kinase, and phosphotidylinosital-3-kinase. In a preferred embodiment the receptor element is a heterodimer (other than LFA-1) composed of a 0 integrin and an a integrin chosen from the following integrins; β1, β2, β3, β4, β5, β6, α1, α2, α3, α4, α5, and α6, or is MAC-1 (β and cd11b), or αVβ3.

In a preferred embodiment the element is an intracellular adhesion molecule (ICAM). ICAMs-1, -2, and -3 are cellular adhesion molecules belonging to the immunogloblin superfamily. Each of these receptors has a single membrane-spanning domain and all bind to [32 integrins via extracellular binding domains similar in structure to Ig-loops. (Signal Transduction, Gomperts, et al., eds, Academic Press Publishers, 2002, Chapter 14, pp 318-319).

In another embodiment the activatable elements cluster for signaling by contact with other surface molecules. In contrast to the receptors discussed above, these elements cluster for signaling by contact with other surface molecules, and generally use molecules presented on the surface of a second cell as ligands. Receptors of this class are important in cell-cell interactions, such mediating cell-to-cell adhesion and immunorecognition. Examples of such receptor elements are CD3 (T cell receptor complex), BCR (B cell receptor complex), CD4, CD28, CD80, CD86, CD54, CD102, CD50 and ICAMs 1, 2 and 3.

In a preferred embodiment the receptor element is a T cell receptor complex (TCR). TCRs occur as either of two distinct heterodimers, αβ, or γ ξ both of which are expressed with the non-polymorphic CD3 polypeptides γΣξ. The CD3 polypeptides, especially ξ and its variants, are critical for intracellular signaling. The αβ TCR heterodimer expressing cells predominate in most lymphoid compartments and are responsible for the classical helper or cytotoxic T cell responses. In most cases, the αβ TCR ligand is a peptide antigen bound to a class I or a class II MHC molecule (Fundamental Immunology, fourth edition, W. E. Paul, ed., Lippincott-Raven Publishers, 1999, Chapter 10, pp 341-367).

In another embodiment, the activatable element is a member of the large family of G-protein-coupled receptors. It has recently been reported that a G-protein-coupled receptors are capable of clustering. (Kroeger, et al., J Biol Chem 276:16, 12736-12743, Apr. 20, 2001; Bai, et al., J Biol Chem 273:36, 23605-23610, Sep. 4, 1998; Rocheville, et al., J Biol Chem 275 (11), 7862-7869, Mar. 17, 2000). As used herein G-protein-coupled receptor, and grammatical equivalents thereof, refers to the family of receptors that bind to heterotrimeric “G proteins.” Many different G proteins are known to interact with receptors. G protein signaling systems include three components: the receptor itself, a GTP-binding protein (G protein), and an intracellular target protein. The cell membrane acts as a switchboard. Messages arriving through different receptors can produce a single effect if the receptors act on the same type of G protein. On the other hand, signals activating a single receptor can produce more than one effect if the receptor acts on different kinds of G proteins, or if the G proteins can act on different effectors.

In their resting state, the G proteins, which consist of alpha (α), beta (β) and gamma (γ) subunits, are complexed with the nucleotide guanosine diphosphate (GDP) and are in contact with receptors. When a hormone or other first messenger binds to a receptor, the receptor changes conformation and this alters its interaction with the G protein. This spurs a subunit to release GDP, and the more abundant nucleotide guanosine triphosphate (GTP), replaces it, activating the G protein. The G protein then dissociates to separate the a subunit from the still complexed beta and gamma subunits. Either the Gα subunit, or the Gβγ complex, depending on the pathway, interacts with an effector. The effector (which is often an enzyme) in turn converts an inactive precursor molecule into an active “second messenger,” which may diffuse through the cytoplasm, triggering a metabolic cascade. After a few seconds, the Gα converts the GTP to GDP, thereby inactivating itself. The inactivated Gα may then reassociate with the Gβγ complex.

Hundreds, if not thousands, of receptors convey messages through heterotrimeric G proteins, of which at least 17 distinct forms have been isolated. Although the greatest variability has been seen in a subunit, several different β and γ structures have been reported. There are, additionally, many different G protein-dependent effectors.

Most G protein-coupled receptors are comprised of a single protein chain that passes through the plasma membrane seven times. Such receptors are often referred to as seven-transmembrane receptors (STRs). More than a hundred different STRs have been found, including many distinct receptors that bind the same ligand, and there are likely many more STRs awaiting discovery.

In addition, STRs have been identified for which the natural ligands are unknown; these receptors are termed “orphan” G protein-coupled receptors, as described above. Examples include receptors cloned by Neote et al. (1993) Cell 72, 415; Kouba et al. FEBS Lett. (1993)321, 173; and Birkenbach et al. (1993) J. Virol. 67, 2209.

Known ligands for G protein coupled receptors include: purines and nucleotides, such as adenosine, cAMP, ATP, UTP, ADP, melatonin and the like; biogenic amines (and related natural ligands), such as 5-hydroxytryptamine, acetylcholine, dopamine, adrenaline, histamine, noradrenaline, tyramine/octopamine and other related compounds; peptides such as adrenocorticotrophic hormone (acth), melanocyte stimulating hormone (msh), melanocortins, neurotensin (nt), bombesin and related peptides, endothelins, cholecystokinin, gastrin, neurokinin b (nk3), invertebrate tachykinin-like peptides, substance k (nk2), substance p (nk1), neuropeptide y (npy), thyrotropin releasing-factor (trf), bradykinin, angiotensin ii, beta-endorphin, c5a anaphalatoxin, calcitonin, chemokines (also called intercrines), corticotrophic releasing factor (crf), dynorphin, endorphin, fmlp and other formylated peptides, follitropin (fsh), fungal mating pheromones, galanin, gastric inhibitory polypeptide receptor (gip), glucagon-like peptides (glps), glucagon, gonadotropin releasing hormone (gnrh), growth hormone releasing hormone(ghrh), insect diuretic hormone, interleukin-8, leutropin (1 h/hcg), met-enkephalin, opioid peptides, oxytocin, parathyroid hormone (pth) and pthrp, pituitary adenylyl cyclase activating peptide (pacap), secretin, somatostatin, thrombin, thyrotropin (tsh), vasoactive intestinal peptide (vip), vasopressin, vasotocin; eicosanoids such as ip-prostacyclin, pg-prostaglandins, tx-thromboxanes; retinal based compounds such as vertebrate 11-cis retinal, invertebrate 11-cis retinal and other related compounds; lipids and lipid-based compounds such as cannabinoids, anandamide, lysophosphatidic acid, platelet activating factor, leukotrienes and the like; excitatory amino acids and ions such as calcium ions and glutamate.

In some embodiments, one or more JAK/STAT regulatory proteins can be simultaneously or sequentially analyzed with other activatable elements. In some embodiments, the activity of the JAK/STAT regulatory protein can be analyzed with another activatable element. In other embodiments, the expression level of a JAK/STAT regulatory protein can be analyzed with another activatable element. In yet another embodiment, the activity and expression level of a JAK/STAT regulatory protein can be analyzed with another activatable element. For example, the activity and expression level of a JAK/STAT regulatory protein can be analyzed simultaneously with the activity level of a gain-of-function mutation of a JAK/STAT pathway component. By analyzing activity and/or expression level of a JAK/STAT regulatory protein with the activity level of a JAK/STAT pathway component, a correlation can be made to determine if there has been a break in regulation activity of the JAK/STAT pathway component.

In some embodiments, analysis of activity and/or expression level of a JAK/STAT regulatory protein with the activity level of a JAK/STAT pathway component provides an improved method for analyzing the effect of a compound on the JAK/STAT signaling pathway, and in particular, the effect of a compound on the JAK/STAT pathway component.

In one embodiment, Jak2 regulatory proteins can be analyzed. The signaling pathways activated by Jaks are tightly regulated at multiple levels by molecules, including but not limited to, protein tyrosine kinases, protein tyrosine phosphatases, ubiquitin ligases, including but not limited to, suppressors of cytokine signaling (SOCS), adaptor proteins and protein inhibitors of activated STATs. These molecules could represent targets for therapeutic intervention in MPNs as well as in other malignancies where the JAK/STAT axis is perturbed.

Lnk is a Jak2 regulatory protein to be measured. Animal model studies demonstrated that Lnk acts as a broad inhibitor of signaling pathways in hematopoietic lineages. Lnk belongs to a family of adaptor proteins comprised of (from the N-terminus) a proline rich domain, a pleckstrin homology domain, a Src homology 2 (SH2) domain and a conserved tyrosine within the C-terminal domain. In murine systems, the Lnk SH2 domain binds tyrosine-phosphorylated signaling molecules, including but not limited to, Jak2, which is necessary for Lnk-mediated negative regulation of cytokine receptors (i.e. Mpl, EpoR, c-KIT, IL-3R, and IL-7R). As a negative regulator of these signaling pathways, Lnk plays a critical role in hematopoiesis by regulating hematopoietic stem cell self renewal, megakaryocytopoiesis and erythropoiesis. Therefore, inhibition of the binding of Lnk to cytokine receptors might lead to enhanced downstream signaling of the receptor and thereby to increased hematopoiesis in response to exposure to cytokines (i.e. erythropoietin in anemic patients). (Gueller et al, Adaptor protein Lnk associates with Y568 in c-Kit. 1: Biochem J. 2008 Jun. 30.) Lnk's mechanism of action in regulating these hematopoietic processes is thought to occur through binding and subsequent negative regulation of Jak activity. Lnk can also bind and inhibit the activity of Jak-2(V617F) suggesting that in MPNs, a diminished function of Lnk, however determined, could provide an alternative mechanism in which to increase Jak-2 activity. (Bersenev et al., Lnk controls mouse hematopoietic stem cell self-renewal and quiescence through direct interactions with Jak2, (J. Clinical Investigation, May 27, 2008, 118(8): 2832-2844). It has been shown that overexpression of Lnk in Ba/F3-MPLW515L cells inhibits cytokine-independent growth, while suppression of Lnk in UT7-MPLW515L cells enhances proliferation. Lnk blocks the activation of Jak2, Stat3, Erk, and Akt in these cells. (Gery et al., Adaptor protein Lnk negatively regulates the mutant MPL, MPLW515L associated with myeloproliferative disorders, Blood, 1 Nov. 2007, Vol. 110, No. 9, pp. 3360-3364.) Thus, Lnk is an important protein to analyze for the evaluation of MPNs.

SOCS3 is a Jak2 regulatory protein to be measured. As mentioned above, Jak2 is negatively regulated by SOCS proteins. However, it was recently reported that Jak2 (V617F) cannot be regulated by SOCS3 and that its activation was actually potentiated in the presence of SOCS3. This correlated with marked tyrosine phosphorylation of SOCS3 protein. These findings suggested that Jak2 V617F has overcome normal SOCS3 regulation by hyperphosphorylating SOCS3, rendering it unable to inhibit the mutant kinase. Thus, Jak2 (V617F) may even exploit SOCS3 to potentiate its myeloproliferative capacity.

SH2-B is a Jak2 regulatory protein to be measured. In contrast to Lnk and SOCS3, SH2-B, another member of this adaptor family, enhances Jak2 activity and acts as a positive regulator of Jak2 function, thus representing another mechanism by which Jak2 can become activated in a mutation independent manner. JAK-2 activity can be modulated through mutations in its JH2 domain and by levels and activity of Lnk, SH2-B and SOCS3. This will have a profound effect on how MPNs are diagnosed and treated and whether the way in which JAK2 is activated will segregate patients into how their disease is managed by JAK-2 inhibitors. These approaches will also be applicable to other diseases where the JAK-2 pathway is deregulated.

In one embodiment, the activatable elements are intracellular receptors capable of clustering. Elements of this class are not membrane-bound. Instead, they are free to diffuse through the intracellular matrix where they bind soluble ligands prior to clustering and signal transduction. In contrast to the previously described elements, many members of this class are capable of binding DNA after clustering to directly affect changes in RNA transcription.

In another embodiment the intracellular receptors capable of clustering are perioxisome proliferator-activated receptors (PPAR). PPARs are soluble receptors responsive to lipophillic compounds, and induce various genes involved in fatty acid metabolism. The three PPAR subtypes, PPAR α, β and γ have been shown to bind to DNA after ligand binding and heterodimerization with retinoid X receptor. (Summanasekera, et al., J Biol Chem, M211261200, Dec. 13, 2002.)

In another embodiment the activatable element is a nucleic acid. Activation and deactivation of nucleic acids can occur in numerous ways including, but not limited to, cleavage of an inactivating leader sequence as well as covalent or non-covalent modifications that induce structural or functional changes. For example, many catalytic RNAs, e.g. hammerhead ribozymes, can be designed to have an inactivating leader sequence that deactivates the catalytic activity of the ribozyme until cleavage occurs. An example of a covalent modification is methylation of DNA. Deactivation by methylation has been shown to be a factor in the silencing of certain genes, e.g. STAT regulating SOCS genes in lymphomas. See Leukemia. See February 2004; 18(2): 356-8. SOCS1 and SHP1 hypermethylation in mantle cell lymphoma and follicular lymphoma: implications for epigenetic activation of the Jak/STAT pathway. Chim C S, Wong K Y, Loong F, Srivastava G.

In another embodiment, the activatable element is a microRNA. MicroRNAs (miRNAs) are non-coding RNA molecules, approximately 22 nucleotides in length, which play important regulatory roles in gene expression in animals and plants. MiRNAs modulate gene flow through post-transcriptional gene silencing through the RNA interference pathway. Once one strand of miRNA is incorporated into the RNA induced silencing complex (RISC), it interacts with the 3′ untranslated regions (UTRs) of target mRNAs through partial sequence complementarity to bring about translational repression or mRNA degradation. The net effect is to downregulate the expression of the target gene by preventing the protein product from being produced. Mirnezami et al., MicroRNAs: Key players in carcinogenesis and novel therapeutic agents, Eur. J. Surg. Oncol., Jun. 9, 2006, doi:10.1016/j.ejso.2008.06.006, hereby fully incorporated by reference in its entirety.

The discovery of a novel class of gene regulators, named microRNAs (miRNAs), has changed the landscape of human genetics. miRNAs are ˜22 nucleotide non-coding RNA that regulate gene expression by binding to 3′ untranslated regions of mRNA. If there is perfect complementarity, the mRNA is cleaved and degraded whereas translational silencing is the main mechanism when base pairing is imperfect. Recent work has led to an increased understanding of the role of miRNAs in hematopoietic differentiation and leukemogenesis. Using animal models engineered to overexpress miR-150, miR-17 approximately 92 and miR-155 or to be deficient for miR-223, miR-155 and miR-17 approximately 92 expression, several groups have now shown that miRNAs are critical for B-lymphocyte development (miR-150 and miR-17 approximately 92), granulopoiesis (miR-223), immune function (miR-155) and B-lymphoproliferative disorders (miR-155 and miR-17 approximately 92). Distinctive miRNA signatures have been described in association with cytogenetics and outcome in acute myeloid leukemia. There is now strong evidence that miRNAs modulate not only hematopoietic differentiation and proliferation but also activity of hematopoietic cells, in particular those related to immune function. Extensive miRNA deregulation has been observed in leukemias and lymphomas and mechanistic studies support a role for miRNAs in the pathogenesis of these disorders (Garzon et al, MicroRNAs in normal and malignant hematopoiesis, Current Opinion Hematology, 2008, 15:352-8). miRNAs regulate critical cellular processes such as cell cycle, apoptosis and differentiation. Consequently impairments in their regulation of these functions through changes in miRNA expression can lead to tumorigenesis. miRNAs can act as oncogenes or tumor suppressors. miRNA profiles can provide important prognostic information as recently shown for acyute myeloid leukemia (Marcucci et al., J. Clinical Oncology (2008) 26:p 5078). In another study, Cimmino et al., (PNAS (2005) 102:p. 13944) showed that patients with chronic lymphocytic leukemia (CLL) have deletions or down regulation of two clustered miRNA genes; mir-15a and mir-16-1. These miRNAs negatively regulate the anti-apoptotic protein Bcl-2 that is often overexpressed in multiple malignancies including but not limited to leukemias and lymphomas. Thus, miRNAs are a potentially useful diagnostic tool in diagnosing cancer, classifying different types of tumors, and determining clinical outcome, including but not limited to, MPNs. A. Esquela-Kerscher and F. J. Slack, Oncomirs—microRNAs with a role in cancer, Nat. Rev. Cancer, April 2006, 6: 259-269 is hereby fully incorporated by reference.

In another embodiment the activatable element is a small molecule, carbohydrate, lipid or other naturally occurring or synthetic compound capable of having an activated isoform. In addition, as pointed out above, activation of these elements need not include switching from one form to another, but can be detected as the presence or absence of the compound. For example, activation of cAMP (cyclic adenosine mono-phosphate) can be detected as the presence of cAMP rather than the conversion from non-cyclic AMP to cyclic AMP.

Examples of proteins that may include activatable elements include, but are not limited to kinases, phosphatases, lipid signaling molecules, adaptor/scaffold proteins, cytokines, cytokine regulators, ubiquitination enzymes, adhesion molecules, cytoskeletal/contractile proteins, heterotrimeric G proteins, small molecular weight GTPases, guanine nucleotide exchange factors, GTPase activating proteins, caspases, proteins involved in apoptosis, cell cycle regulators, molecular chaperones, metabolic enzymes, vesicular transport proteins, hydroxylases, isomerases, deacetylases, methylases, demethylases, tumor suppressor genes, proteases, ion channels, molecular transporters, transcription factors/DNA binding factors, regulators of transcription, and regulators of translation. Examples of activatable elements, activation states and methods of determining the activation level of activatable elements are described in US Publication Number 20060073474 entitled “Methods and compositions for detecting the activation state of multiple proteins in single cells” and US Publication Number 20050112700 entitled “Methods and compositions for risk stratification” the content of which are incorporate here by reference. See also U.S. Ser. Nos. 61/048,886; 61/048,920; and Shulz et al., Current Protocols in Immunology 2007, 78:8.17.1-20.

In some embodiments, the protein is selected from the group consisting of HER receptors, PDGF receptors, Kit receptor, FGF receptors, Eph receptors, Trk receptors, IGF receptors, Insulin receptor, Met receptor, Ret, VEGF receptors, TIE1, TIE2, FAK, Jak1, Jak2, Jak3, Tyk2, Src, Lyn, Fyn, Lck, Fgr, Yes, Csk, Abl, Btk, ZAP70, Syk, IRAKs, cRaf, ARaf, BRAF, Mos, Lim kinase, ILK, Tpl, ALK, TGFβ receptors, BMP receptors, MEKKs, ASK, MLKs, DLK, PAKs, Mek 1, Mek 2, MKK3/6, MKK4/7, ASK1, Cot, NIK, Bub, Myt 1, Weel, Casein kinases, PDK1, SGK1, SGK2, SGK3, Akt1, Akt2, Akt3, p90Rsks, p70S6 Kinase, Prks, PKCs, PKAs, ROCK 1, ROCK 2, Auroras, CaMKs, MNKs, AMPKs, MELK, MARKs, Chk1, Chk2, LKB-1, MAPKAPKs, Pim1, Pim2, Pim3, IKKs, Cdks, Jnks, Erks, IKKs, GSK3α, GSK3β, Cdks, CLKs, PKR, PI3-Kinase class 1, class 2, class 3, mTor, SAPK/JNK1,2,3, p38s, PKR, DNA-PK, ATM, ATR, Receptor protein tyrosine phosphatases (RPTPs), LAR phosphatase, CD45, Non receptor tyrosine phosphatases (NPRTPs), SHPs, MAP kinase phosphatases (MKPs), Dual Specificity phosphatases (DUSPs), CDC25 phosphatases, Low molecular weight tyrosine phosphatase, Eyes absent (EYA) tyrosine phosphatases, Slingshot phosphatases (SSH), serine phosphatases, PP2A, PP2B, PP2C, PP1, PP5, inositol phosphatases, PTEN, SHIPs, myotubularins, phosphoinositide kinases, phopsholipases, prostaglandin synthases, 5-lipoxygenase, sphingosine kinases, sphingomyelinases, adaptor/scaffold proteins, Shc, Grb2, BLNK, LAT, B cell adaptor for PI3-kinase (BCAP), SLAP, Dok, KSR, MyD88, Crk, CrkL, GAD, Nck, Grb2 associated binder (GAB), Fas associated death domain (FADD), TRADD, TRAF2, RIP, T-Cell leukemia family, IL-2, IL-4, IL-8, IL-6, interferon β, interferon α, suppressors of cytokine signaling (SOCs), Cbl, SCF ubiquitination ligase complex, APC/C, adhesion molecules, integrins, Immunoglobulin-like adhesion molecules, selectins, cadherins, catenins, focal adhesion kinase, p130CAS, fodrin, actin, paxillin, myosin, myosin binding proteins, tubulin, eg5/KSP, CENPs, β-adrenergic receptors, muscarinic receptors, adenylyl cyclase receptors, small molecular weight GTPases, H-Ras, K-Ras, N-Ras, Ran, Rac, Rho, Cdc42, Arfs, RABs, RHEB, Vav, Tiam, Sos, Dbl, PRK, TSC1,2, Ras-GAP, Arf-GAPs, Rho-GAPs, caspases, Caspase 2, Caspase 3, Caspase 6, Caspase 7, Caspase 8, Caspase 9, Bc1-2, Mc1-1, Bcl-XL, Bel-w, Bel-B, Al, Bax, Bak, Bok, Bik, Bad, Bid, Bim, Bmf, Hrk, Noxa, Puma, IAPs, XIAP, Smac, Cdk4, Cdk 6, Cdk 2, Cdk1, Cdk 7, Cyclin D, Cyclin E, Cyclin A, Cyclin B, Rb, p16, pl4Arf, p27KIP, p21CIP, molecular chaperones, Hsp90s, Hsp70, Hsp27, metabolic enzymes, Acetyl-CoAa Carboxylase, ATP citrate lyase, nitric oxide synthase, caveolins, endosomal sorting complex required for transport (ESCRT) proteins, vesicular protein sorting (Vsps), hydroxylases, prolyl-hydroxylases PHD-1, 2 and 3, asparagine hydroxylase FIH transferases, Pinl prolyl isomerase, topoisomerases, deacetylases, Histone deacetylases, sirtuins, histone acetylases, CBP/P300 family, MYST family, ATF2, DNA methyl transferases, Histone H3K4 demethylases, H31(27, JHDM2A, UTX, VHL, WT-1, p53, Hdm, PTEN, ubiquitin proteases, urokinase-type plasminogen activator (uPA) and uPA receptor (uPAR) system, cathepsins, metalloproteinases, esterases, hydrolases, separase, potassium channels, sodium channels, multi-drug resistance proteins, P-Gycoprotein, nucleoside transporters, Ets, Elk, SMADs, Rel-A (p65-NFKB), CREB, NFAT, ATF-2, AFT, Myc, Fos, Spl, Egr-1, T-bet, β-catenin, HIFs, FOXOs, E2Fs, SRFs, TCFs, Egr-1, β-catenin, FOXO STAT1, STAT 3, STAT 4, STAT 5, STAT 6, p53, WT-1, HMGA, pS6, 4EPB-1, eIF4E-binding protein, RNA polymerase, initiation factors, elongation factors.

Generally, the methods of the invention involve determining the activation levels of an activatable element in a plurality of single cells in a sample. The activation levels can be obtained by perturbing the cell state using a modulator.

### Drug Transporters

A key issue in the treatment of many cancers is the development of resistance to chemotherapeutic drugs. Of the many resistance mechanisms, two classes of transporters play a major role. Of the many resistance mechanisms, two classes of transporters play a major role: 1) human ATP-binding cassette (ABC) superfamily of proteins; 2) Concentrative and Equilibrative Nucleoside Transporters (CNT and ENT, respectively). For further discussion, see U.S. Patent Application 61/085,789.

In some embodiments, analysis of one or more drug transporters can be simultaneously or sequentially analyzed with activatable elements as described above. In some embodiments, analysis of one or more drug transporters with the activity level of a JAK/STAT pathway component provides an improved method for analyzing the effect of a compound on the JAK/STAT signaling pathway. Since a drug transporter mechanism can have an effect on the ability of a compound to function (e.g. the drug transporter can pump the compound out of the cell), correlation of activity of a drug transporter with analysis of the activity level of a JAK/STAT pathway component can provide additional information on the efficacy of the compound.

### Modulators

In some embodiments, the methods and composition utilize a modulator. A modulator can be an activator, a therapeutic compound, an inhibitor or a compound capable of impacting a cellular pathway. Modulators can also take the form of a variety of environmental cues and inputs.

Modulation can be performed in a variety of environments. In some embodiments, cells are exposed to a modulator immediately after collection. In some embodiments where there is a mixed population of cells, purification of cells is performed after modulation. In some embodiments, whole blood is collected to which a modulator is added. In some embodiments, cells are modulated after processing for single cells or purified fractions of single cells. As an illustrative example, whole blood can be collected and processed for an enriched fraction of lymphocytes that is then exposed to a modulator. Modulation can include exposing cells to more than one modulator. For instance, in some embodiments, cells are exposed to at least 2, 3, 4, 5, 6, 7, 8, 9, or 10 modulators. See U.S. Patent Application 61/048,657 which is incorporated by reference.

In some embodiments, cells are cultured post collection in a suitable media before exposure to a modulator. In some embodiments, the media is a growth media. In some embodiments, the growth media is a complex media that may include serum. In some embodiments, the growth media comprises serum. In some embodiments, the serum is selected from the group consisting of fetal bovine serum, bovine serum, human serum, porcine serum, horse serum, and goat serum. In some embodiments, the serum level ranges from 0.0001% to 30%. In some embodiments, the growth media is a chemically defined minimal media and is without serum. In some embodiments, cells are cultured in a differentiating media.

Modulators include chemical and biological entities, and physical or environmental stimuli. Modulators can act extracellularly or intracellularly. Chemical and biological modulators include growth factors, cytokines, drugs, immune modullators, ions, neurotransmitters, adhesion molecules, hormones, small molecules, inorganic compounds, polynucleotides, antibodies, natural compounds, lectins, lactones, chemotherapeutic agents, biological response modifiers, carbohydrates, proteases and free radicals. Modulators include complex and undefined biologic compositions that may comprise cellular or botanical extracts, cellular or glandular secretions, physiologic fluids such as serum, amniotic fluid, or venom. Physical and environmental stimuli include electromagnetic, ultraviolet, infrared or particulate radiation, redox potential and pH, the presence or absences of nutrients, changes in temperature, changes in oxygen partial pressure, changes in ion concentrations and the application of oxidative stress. Modulators can be endogenous or exogenous and may produce different effects depending on the concentration and duration of exposure to the single cells or whether they are used in combination or sequentially with other modulators. Modulators can act directly on the activatable elements or indirectly through the interaction with one or more intermediary biomolecule. Indirect modulation includes alterations of gene expression wherein the expressed gene product is the activatable element or is a modulator of the activatable element.

In some embodiments, the modulator is an activator. In some embodiments the modulator is an inhibitor. In some embodiments, cells are exposed to one or more modulators. In some embodiments, cells are exposed to at least 2, 3, 4, 5, 6, 7, 8, 9, or 10 modulators. In some embodiments, cells are exposed to at least two modulators, wherein one modulator is an activator and one modulator is an inhibitor. In some embodiments, cells are exposed to at least 2, 3, 4, 5, 6, 7, 8, 9, or 10 modulators, where at least one of the modulators is an inhibitor.

In some embodiments, the cross-linker is a molecular binding entity. In some embodiments, the molecular binding entity is a monovalent, bivalent, or multivalent is made more multivalent by attachment to a solid surface or tethered on a nanoparticle surface to increase the local valency of the epitope binding domain.

In some embodiments, the inhibitor is an inhibitor of a cellular factor or a plurality of factors that participates in a cellular pathway (e.g. signaling cascade) in the cell. In some embodiments, the inhibitor is a phosphatase inhibitor.

In some embodiments, the activation level of an activatable element in a cell is determined by contacting the cell with an inhibitor and a modulator, where the modulator can be an inhibitor or an activator. In some embodiments, the activation level of an activatable element in a cell is determined by contacting the cell with an inhibitor and an activator. In some embodiments, the activation level of an activatable element in a cell is determined by contacting the cell with two or more modulators.

In some embodiments, the invention can be used to analyze the modulators, pathways, and associated cell sub-sets listed in Table 7. These modulators, pathways, and cell sub-sets are given by way of example only, and do not limit the invention.

### Gating

In some embodiments of the invention, different gating strategies can be used in order to analyze only blasts in the sample of mixed population after treatment with the modulator. These gating strategies can be based on the presence of one or more specific surface marker expressed on each cell type. See U.S. Patent Applications No. 61/265,743, 61/120,320, and 61/079,766, are hereby incorporated by reference.

### (b) Detection

In practicing the methods of this invention, the detection of the status of the one or more activatable elements can be carried out by a person, such as a technician in the laboratory. Alternatively, the detection of the status of the one or more activatable elements can be carried out using automated systems. In either case, the detection of the status of the one or more activatable elements for use according to the methods of this invention is performed according to standard techniques and protocols well-established in the art.

One or more activatable elements can be detected and/or quantified by any method that detect and/or quantitates the presence of the activatable element of interest. Such methods may include radioimmunoassay (RIA) or enzyme linked immunoabsorbance assay (ELISA), immunohistochemistry, immunofluorescent histochemistry with or without confocal microscopy, reversed phase assays, homogeneous enzyme immunoassays, and related non-enzymatic techniques, Western blots, whole cell staining, immunoelectronmicroscopy, nucleic acid amplification, gene array, protein array, mass spectrometry, patch clamp, 2-dimensional gel electrophoresis, differential display gel electrophoresis, microsphere-based multiplex protein assays, label-free cellular assays and flow cytometry, etc. U.S. Pat. No. 4,568,649 describes ligand detection systems, which employ scintillation counting. These techniques are particularly useful for modified protein parameters. Cell readouts for proteins and other cell determinants can be obtained using fluorescent or otherwise tagged reporter molecules. Flow cytometry methods are useful for measuring intracellular parameters. See the above patents and applications for example methods.

In some embodiments, the present invention provides methods for determining an activatable element's activation profile for a single cell. The methods may comprise analyzing cells by flow cytometry on the basis of the activation level of at least two activatable elements. Binding elements (e.g. activation state-specific antibodies) are used to analyze cells on the basis of activatable element activation level, and can be detected as described below. Alternatively, non-binding elements systems as described above can be used in any system described herein.

Detection of cell signaling states may be accomplished using binding elements and labels. Cell signaling states may be detected by a variety of methods known in the art. They generally involve a binding element, such as an antibody, and a label, such as a fluorchrome to form a detection element (sometimes called a stain). Detection elements do not need to have both of the above agents, but can be one unit that possesses both qualities. These and other methods are well described in U.S. Pat. Nos. 7,381,535 and 7,393,656 and U.S. Ser. Nos. 61/265,743, 10/193,462; 11/655,785; 11/655,789; 11/655,821; 11/338,957, 61/048,886; 61/048,920; and 61/048,657 which are all incorporated by reference in their entireties.

In one embodiment of the invention, it is advantageous to increase the signal to noise ratio by contacting the cells with the antibody and label for a time greater than 5, 6, 7, 8, 9, 10, 11, 12, 13, 14, 15, 16, 17, 18, 19, 20, 24 or up to 48 or more hours.

When using fluorescent labeled components in the methods and compositions of the present invention, it will recognized that different types of fluorescent monitoring systems, e.g., cytometric measurement device systems, can be used to practice the invention. In some embodiments, flow cytometric systems are used or systems dedicated to high throughput screening, e.g. 96 well or greater microtiter plates. Methods of performing assays on fluorescent materials are well known in the art and are described in, e.g., Lakowicz, J. R., Principles of Fluorescence Spectroscopy, New York: Plenum Press (1983); Herman, B., Resonance energy transfer microscopy, in: Fluorescence Microscopy of Living Cells in Culture, Part B, Methods in Cell Biology, vol. 30, ed. Taylor, D. L. & Wang, Y.-L., San Diego: Academic Press (1989), pp. 219-243; Turro, N. J., Modern Molecular Photochemistry, Menlo Park: Benjamin/Cummings Publishing Col, Inc. (1978), pp. 296-361.

Fluorescence in a sample can be measured using a fluorimeter. In general, excitation radiation, from an excitation source having a first wavelength, passes through excitation optics. The excitation optics cause the excitation radiation to excite the sample. In response, fluorescent proteins in the sample emit radiation that has a wavelength that is different from the excitation wavelength. Collection optics then collect the emission from the sample. The device can include a temperature controller to maintain the sample at a specific temperature while it is being scanned. According to one embodiment, a multi-axis translation stage moves a microtiter plate holding a plurality of samples in order to position different wells to be exposed. The multi-axis translation stage, temperature controller, auto-focusing feature, and electronics associated with imaging and data collection can be managed by an appropriately programmed digital computer. The computer also can transform the data collected during the assay into another format for presentation. In general, known robotic systems and components can be used.

Other methods of detecting fluorescence may also be used, e.g., Quantum dot methods (see, e.g., Goldman et al., J. Am. Chem. Soc. (2002) 124:6378-82; Pathak et al. J. Am. Chem. Soc. (2001) 123:4103-4; and Remade et al., Proc. Natl. Sci. USA (2000) 18:553-8, each expressly incorporated herein by reference) as well as confocal microscopy. In general, flow cytometry involves the passage of individual cells through the path of a laser beam. The scattering the beam and excitation of any fluorescent molecules attached to, or found within, the cell is detected by photomultiplier tubes to create a readable output, e.g. size, granularity, or fluorescent intensity.

The detecting, sorting, or isolating step of the methods of the present invention can entail fluorescence-activated cell sorting (FACS) techniques, where FACS is used to select cells from the population containing a particular surface marker, or the selection step can entail the use of magnetically responsive particles as retrievable supports for target cell capture and/or background removal. A variety of FACS systems are known in the art and can be used in the methods of the invention (see e.g., W099/54494, filed Apr. 16, 1999; U.S. Ser. No. 20010006787, filed Jul. 5, 2001, each expressly incorporated herein by reference).

In some embodiments, a FACS cell sorter (e.g. a FACSVantage™ Cell Sorter, Becton Dickinson Immunocytometry Systems, San Jose, Calif.) is used to sort and collect cells based on their activation profile (positive cells) in the presence or absence of an increase in activation level in an activatable element in response to a modulator. Other flow cytometers that are commercially available include the LSR II and the Canto II both available from Becton Dickinson. See Shapiro, Howard M., Practical Flow Cytometry, 4th Ed., John Wiley & Sons, Inc., 2003 for additional information on flow cytometers.

In some embodiments, the cells are first contacted with fluorescent-labeled activation state-specific binding elements (e.g. antibodies) directed against specific activation state of specific activatable elements. In such an embodiment, the amount of bound binding element on each cell can be measured by passing droplets containing the cells through the cell sorter. By imparting an electromagnetic charge to droplets containing the positive cells, the cells can be separated from other cells. The positively selected cells can then be harvested in sterile collection vessels. These cell-sorting procedures are described in detail, for example, in the FACSVantage™. Training Manual, with particular reference to sections 3-11 to 3-28 and 10-1 to 10-17, which is hereby incorporated by reference in its entirety. See the patents, applications and articles referred to, and incorporated above for detection systems.

Fluorescent compounds such as Daunorubicin and Enzastaurin are problematic for flow cytometry based biological assays due to their broad fluorescence emission spectra. These compounds get trapped inside cells after fixation with agents like paraformaldehyde, and are excited by one or more of the lasers found on flow cytometers. The fluorescence emission of these compounds is often detected in multiple PMT detectors which complicates their use in multiparametric flow cytometry. A way to get around this problem is to compensate out the fluorescence emission of the compound from the PMT detectors used to measure the relevant biological markers. This is achieved using a PMT detector with a bandpass filter near the emission maximum of the fluorescent compound, and cells incubated with the compound as the compensation control when calculating a compensation matrix. The cells incubated with the fluorescent compound are fixed with paraformaldehyde, then washed and permeabilized with 100% methanol. The methanol is washed out and the cells are mixed with unlabeled fixed/permed cells to yield a compensation control consisting of a mixture of fluorescent and negative cell populations.

In another embodiment, positive cells can be sorted using magnetic separation of cells based on the presence of an isoform of an activatable element. In such separation techniques, cells to be positively selected are first contacted with specific binding element (e.g., an antibody or reagent that binds an isoform of an activatable element). The cells are then contacted with retrievable particles (e.g., magnetically responsive particles) that are coupled with a reagent that binds the specific element. The cell-binding element-particle complex can then be physically separated from non-positive or non-labeled cells, for example, using a magnetic field. When using magnetically responsive particles, the positive or labeled cells can be retained in a container using a magnetic field while the negative cells are removed. These and similar separation procedures are described, for example, in the Baxter Immunotherapy Isolex training manual which is hereby incorporated in its entirety.

In some embodiments, methods for the determination of a receptor element activation state profile for a single cell are provided. The methods comprise providing a population of cells and analyzing the population of cells by flow cytometry. Preferably, cells are analyzed on the basis of the activation level of at least two activatable elements. In some embodiments, a multiplicity of activatable element activation-state antibodies is used to simultaneously determine the activation level of a multiplicity of elements.

In some embodiments, cell analysis by flow cytometry on the basis of the activation level of at least two elements is combined with a determination of other flow cytometry readouts, such as the presence of surface markers, granularity and cell size to provide a correlation between the activation level of a multiplicity of elements and other cell qualities measurable by flow cytometry for single cells.

In an embodiment, the present invention provides a method for determining selectivity and potency of various compounds by enabling dose-response titration curves to be generated for multiple cell types and multiple cellular pathways simultaneously. In another embodiment, the selectivity and potency of pathway-selective compounds or cell-type specific compounds is determined.

As will be appreciated, the present invention also provides for the ordering of element clustering events in signal transduction. Particularly, the present invention allows the artisan to construct an element clustering and activation hierarchy based on the correlation of levels of clustering and activation of a multiplicity of elements within single cells. Ordering can be accomplished by comparing the activation level of a cell or cell population with a control at a single time point, or by comparing cells at multiple time points to observe subpopulations arising out of the others.

The present invention provides a valuable method of determining the presence of cellular subsets within cellular populations that are either homogenous or heterogeneous. In one embodiment, signal transduction pathways are evaluated in homogeneous cell populations. In homogenous populations variances in signaling between cells usually do not qualitatively nor quantitatively mask signal transduction events and alterations therein. As the ultimate homogeneous system is the single cell, the present invention allows the individual evaluation of cells to allow true differences to be identified in a significant way.

One embodiment of the invention allows one to compare nodes within cell types, subsets, or populations within the same fluid volume, or nodes in different fluid volumes. The words cell types, subsets, or populations may be used to describe groups of different cells which may be placed in a fluid volume and ultimately analyzed separately. As outlined herein, these cellular subsets often exhibit altered biological characteristics, such as basal levels of activation in the absence of a modulator or altered response to the same modulators, when compared to other subsets within the population. Some of the methods of the invention allow the identification of subsets of cells from a population that exhibit different responses as compared with other subsets. In an embodiment of the invention, the methods allow the identification of subsets of cells from a population, such as primary cell populations comprising peripheral blood mononuclear cells that exhibit altered responses associated with presence of a condition, as compared to other subsets. Additionally, this type of evaluation distinguishes between different activation states, altered responses to modulators, cell lineages, cell differentiation states, etc.

As will be appreciated, these methods provide for the identification of distinct signaling cascades for both artificial and stimulatory conditions in complex cell populations, such as peripheral blood mononuclear cells (PMBCs), whole blood, bone marrow, or naive and memory lymphocytes.

When necessary cells are dispersed into a single cell suspension, e.g. by enzymatic digestion with a suitable protease, e.g. collagenase, dispase, etc; and the like, an appropriate solution is used for dispersion or suspension. Such solution will generally be a balanced salt solution, e.g. normal saline, PBS, Hanks balanced salt solution, etc., conveniently supplemented with fetal calf serum or other naturally occurring factors, in conjunction with an acceptable buffer at low concentration, generally from 5-25 mM. Convenient buffers include HEPES 1 phosphate buffers, lactate buffers, etc. The cells may be fixed, e.g. with 3% paraformaldehyde, and are usually permeabilized, e.g. with ice cold methanol; HEPES-buffered PBS containing 0.1% saponin, 3% BSA; covering for 2 mM in acetone at −200 C; and the like as known in the art and according to the methods described herein.

In some embodiments, one or more cells are contained in a well of a 96 well plate or other commercially available multi-well plate. In an alternate embodiment, the reaction mixture or cells are in a cytometric measurement device. Other multi-well plates useful in the present invention include, but are not limited to 384 well plates and 1536 well plates. Still other vessels for containing the reaction mixture or cells and useful in the present invention will be apparent to the skilled artisan.

The addition of the components of the assay for detecting the activation level or activity of an activatable element, and/or modulation of such activation level or activity, may be simultaneous, sequential or in a predetermined order or grouping under conditions appropriate for the activity that is assayed for. Such conditions are described here and known in the art. Moreover, further guidance is provided below (see, e.g., in the Examples).

In some embodiments, the activation level of an activatable element is measured using Inductively Coupled Plasma Mass Spectrometer (ICP-MS). A binding element that has been labeled with a specific element binds to the activatable. When the cell is introduced into the ICP, it is atomized and ionized. The elemental composition of the cell, including the labeled binding element that is bound to the activatable element, is measured. The presence and intensity of the signals corresponding to the labels on the binding element indicates the level of the activatable element on that cell (Tanner et al. Spectrochimica Acta Part B: Atomic Spectroscopy, 2007 March; 62(3):188-195.).

As will be appreciated by one of skill in the art, the instant methods and compositions find use in a variety of other assay formats in addition to flow cytometry analysis. For example, DNA microarrays are commercially available through a variety of sources (Affymetrix, Santa Clara, Calif.) or they can be custom made in the lab using arrayers which are also know (Perkin Elmer). In addition, protein chips and methods for synthesis are known. These methods and materials may be adapted for the purpose of affixing activation state binding elements to a chip in a prefigured array. In some embodiments, such a chip comprises a multiplicity of element activation state binding elements, and is used to determine an element activation state profile for elements present on the surface of a cell.

In some embodiments, a chip comprises a multiplicity of the “second set binding elements,” in this case generally unlabeled. Such a chip is contacted with sample, preferably cell extract, and a second multiplicity of binding elements comprising element activation state specific binding elements is used in the sandwich assay to simultaneously determine the presence of a multiplicity of activated elements in sample. Preferably, each of the multiplicity of activation state-specific binding elements is uniquely labeled to facilitate detection.

In some embodiments, confocal microscopy can be used to detect activation profiles for individual cells. Confocal microscopy relies on the serial collection of light from spatially filtered individual specimen points, which is then electronically processed to render a magnified image of the specimen. The signal processing involved confocal microscopy has the additional capability of detecting labeled binding elements within single cells, accordingly in this embodiment the cells can be labeled with one or more binding elements. In some embodiments the binding elements used in connection with confocal microscopy are antibodies conjugated to fluorescent labels, however other binding elements, such as other proteins or nucleic acids are also possible.

In some embodiments, the methods and compositions of the instant invention can be used in conjunction with an “In-Cell Western Assay.” In such an assay, cells are initially grown in standard tissue culture flasks using standard tissue culture techniques. Once grown to optimum confluency, the growth media is removed and cells are washed and trypsinized. The cells can then be counted and volumes sufficient to transfer the appropriate number of cells are aliquoted into microwell plates (e.g., Nunc™ 96 Microwell™ plates). The individual wells are then grown to optimum confluency in complete media whereupon the media is replaced with serum-free media. At this point controls are untouched, but experimental wells are incubated with a modulator, e.g. EGF. After incubation with the modulator cells are fixed and stained with labeled antibodies to the activation elements being investigated. Once the cells are labeled, the plates can be scanned using an imager such as the Odyssey Imager (LiCor, Lincoln Nebr.) using techniques described in the Odyssey Operator's Manual v1.2., which is hereby incorporated in its entirety. Data obtained by scanning of the multiwell plate can be analyzed and activation profiles determined as described below.

In some embodiments, the detecting is by high pressure liquid chromatography (HPLC), for example, reverse phase HPLC, and in a further aspect, the detecting is by mass spectrometry.

These instruments can fit in a sterile laminar flow or fume hood, or are enclosed, self-contained systems, for cell culture growth and transformation in multi-well plates or tubes and for hazardous operations. The living cells may be grown under controlled growth conditions, with controls for temperature, humidity, and gas for time series of the live cell assays. Automated transformation of cells and automated colony pickers may facilitate rapid screening of desired cells.

Flow cytometry or capillary electrophoresis formats can be used for individual capture of magnetic and other beads, particles, cells, and organisms.

Flexible hardware and software allow instrument adaptability for multiple applications. The software program modules allow creation, modification, and running of methods. The system diagnostic modules allow instrument alignment, correct connections, and motor operations. Customized tools, labware, and liquid, particle, cell and organism transfer patterns allow different applications to be performed. Databases allow method and parameter storage. Robotic and computer interfaces allow communication between instruments.

In some embodiment, the methods of the invention include the use of liquid handling components. The liquid handling systems can include robotic systems comprising any number of components. In addition, any or all of the steps outlined herein may be automated; thus, for example, the systems may be completely or partially automated. See U.S. Patent Application No. 61/048,657 and Ser. No. 12/606,869.

As will be appreciated by those in the art, there are a wide variety of components which can be used, including, but not limited to, one or more robotic arms; plate handlers for the positioning of microplates; automated lid or cap handlers to remove and replace lids for wells on non-cross contamination plates; tip assemblies for sample distribution with disposable tips; washable tip assemblies for sample distribution; 96 well loading blocks; cooled reagent racks; microtiter plate pipette positions (optionally cooled); stacking towers for plates and tips; and computer systems.

Fully robotic or microfluidic systems include automated liquid-, particle-, cell- and organism-handling including high throughput pipetting to perform all steps of screening applications. This includes liquid, particle, cell, and organism manipulations such as aspiration, dispensing, mixing, diluting, washing, accurate volumetric transfers; retrieving, and discarding of pipet tips; and repetitive pipetting of identical volumes for multiple deliveries from a single sample aspiration. These manipulations are cross-contamination-free liquid, particle, cell, and organism transfers. This instrument performs automated replication of microplate samples to filters, membranes, and/or daughter plates, high-density transfers, full-plate serial dilutions, and high capacity operation.

In some embodiments, chemically derivatized particles, plates, cartridges, tubes, magnetic particles, or other solid phase matrix with specificity to the assay components are used. The binding surfaces of microplates, tubes or any solid phase matrices include non-polar surfaces, highly polar surfaces, modified dextran coating to promote covalent binding, antibody coating, affinity media to bind fusion proteins or peptides, surface-fixed proteins such as recombinant protein A or G, nucleotide resins or coatings, and other affinity matrix are useful in this invention.

In some embodiments, platforms for multi-well plates, multi-tubes, holders, cartridges, minitubes, deep-well plates, microcentrifuge tubes, cryovials, square well plates, filters, chips, optic fibers, beads, and other solid-phase matrices or platform with various volumes are accommodated on an upgradable modular platform for additional capacity. This modular platform includes a variable speed orbital shaker, and multi-position work decks for source samples, sample and reagent dilution, assay plates, sample and reagent reservoirs, pipette tips, and an active wash station. In some embodiments, the methods of the invention include the use of a plate reader.

In some embodiments, thermocycler and thermoregulating systems are used for stabilizing the temperature of heat exchangers such as controlled blocks or platforms to provide accurate temperature control of incubating samples from 0° C. to 100° C.

In some embodiments, interchangeable pipet heads (single or multi-channel) with single or multiple magnetic probes, affinity probes, or pipetters robotically manipulate the liquid, particles, cells, and organisms. Multi-well or multi-tube magnetic separators or platforms manipulate liquid, particles, cells, and organisms in single or multiple sample formats.

In some embodiments, the instrumentation will include a detector, which can be a wide variety of different detectors, depending on the labels and assay. In some embodiments, useful detectors include a microscope(s) with multiple channels of fluorescence; plate readers to provide fluorescent, ultraviolet and visible spectrophotometric detection with single and dual wavelength endpoint and kinetics capability, fluorescence resonance energy transfer (FRET), luminescence, quenching, two-photon excitation, and intensity redistribution; CCD cameras to capture and transform data and images into quantifiable formats; and a computer workstation.

In some embodiments, the robotic apparatus includes a central processing unit which communicates with a memory and a set of input/output devices (e.g., keyboard, mouse, monitor, printer, etc.) through a bus. Again, as outlined below, this may be in addition to or in place of the CPU for the multiplexing devices of the invention. The general interaction between a central processing unit, a memory, input/output devices, and a bus is known in the art. Thus, a variety of different procedures, depending on the experiments to be run, are stored in the CPU memory.

These robotic fluid handling systems can utilize any number of different reagents, including buffers, reagents, samples, washes, assay components such as label probes, etc.

### Analysis

Advances in flow cytometry have enabled the individual cell enumeration of fifteen or more simultaneous parameters (De Rosa et al., 2001) and are moving towards the study of genomic and proteomic data subsets (Krutzik and Nolan, 2003; Perez and Nolan, 2002). Likewise, advances in other techniques (e.g. microarrays) allow for the identification of multiple activatable elements. As the number of parameters, epitopes, and samples have increased, the complexity of experiments and the challenges of data analysis have grown rapidly. An additional layer of data complexity has been added by the development of stimulation panels which enable the study of activatable elements under a growing set of experimental conditions. See Krutzik et al, Nature Chemical Biology February 2008. Methods for the analysis of multiple parameters are well known in the art. See U.S. Patent Application No. 61/079,579 for gating analysis.

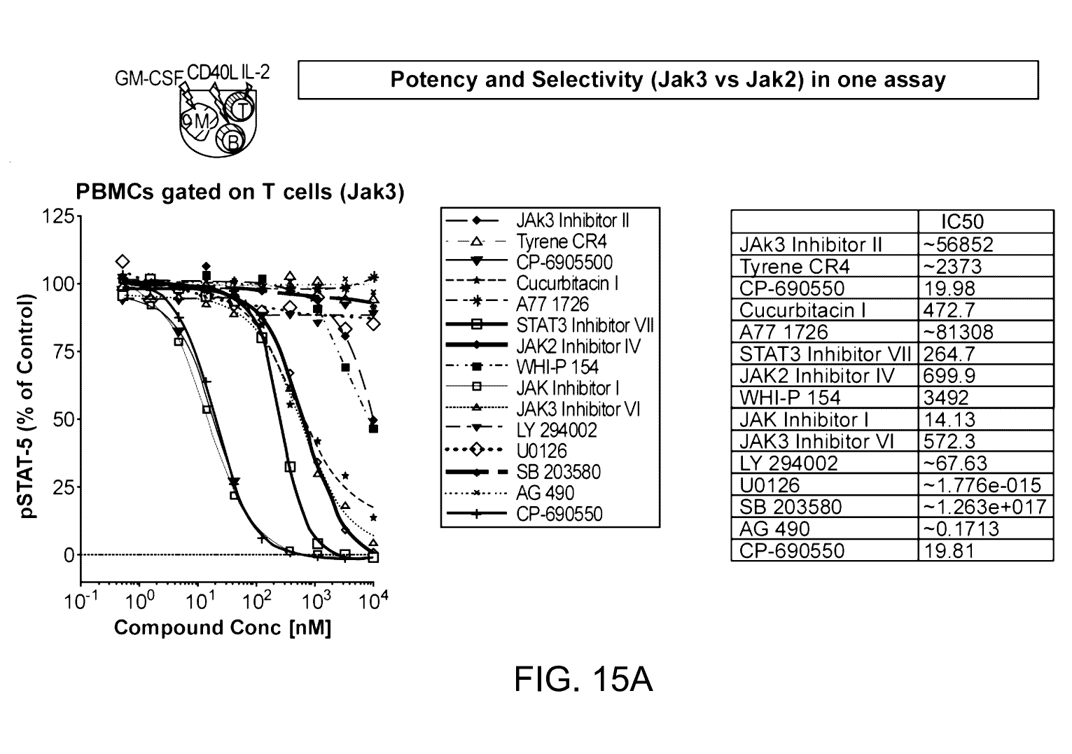

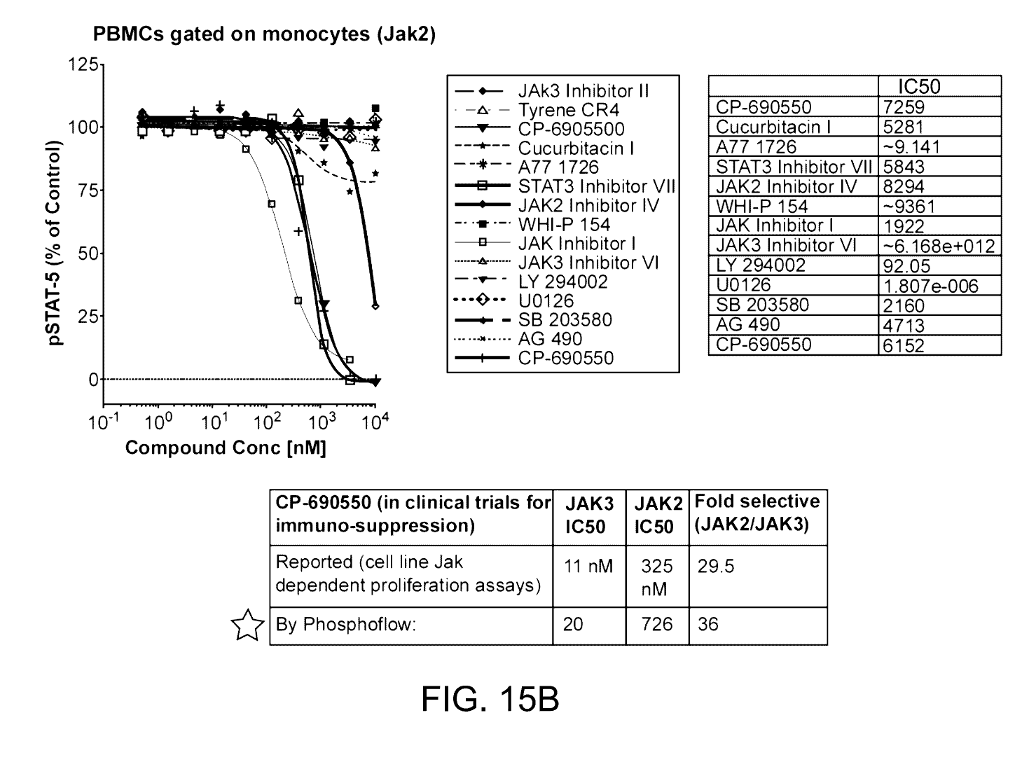

In some embodiments where flow cytometry is used, flow cytometry experiments are performed and the results are expressed as fold changes using graphical tools and analyses, including, but not limited to a heat map or a histogram to facilitate evaluation. One common way of comparing changes in a set of flow cytometry samples is to overlay histograms of one parameter on the same plot. In other embodiments one or more compounds are screened for selectivity for a cell type or cellular pathway, for potency of effects on this pathway and/or cell type, and for off-target effects on other cell types and pathways. Dose-titration experiments may be performed to determine IC₅₀ values for the compound's effects on different pathways or different cell populations. i. In some embodiments, potency and selectivity may be determined in the same assay (See FIG. 15 for an example of such an assay).

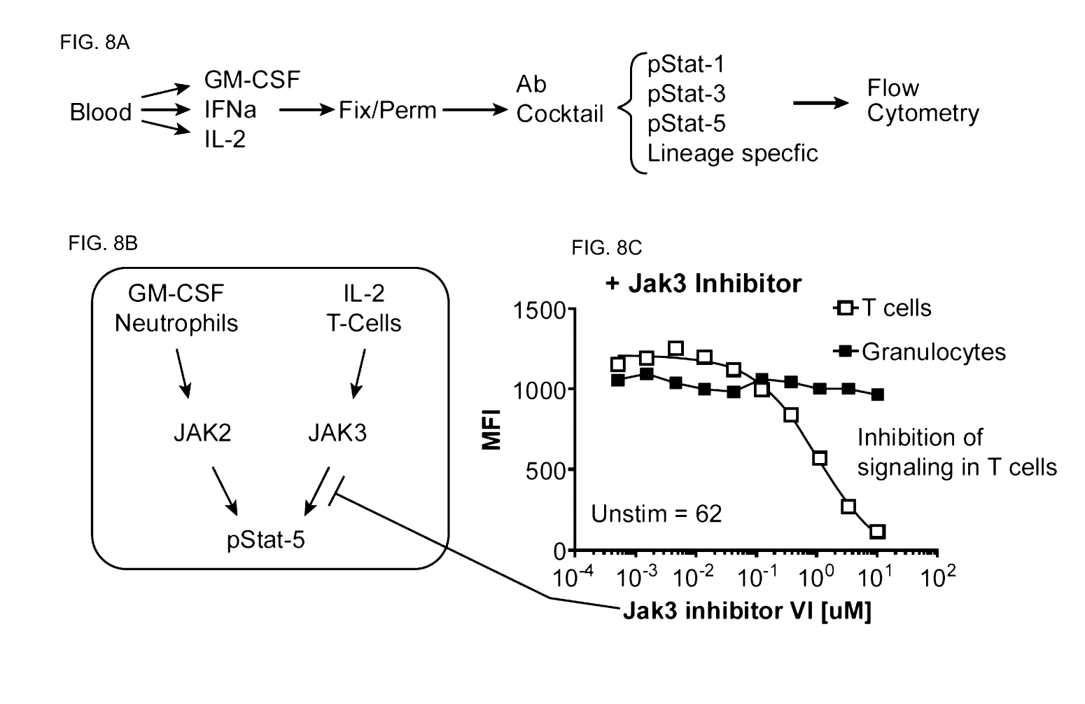

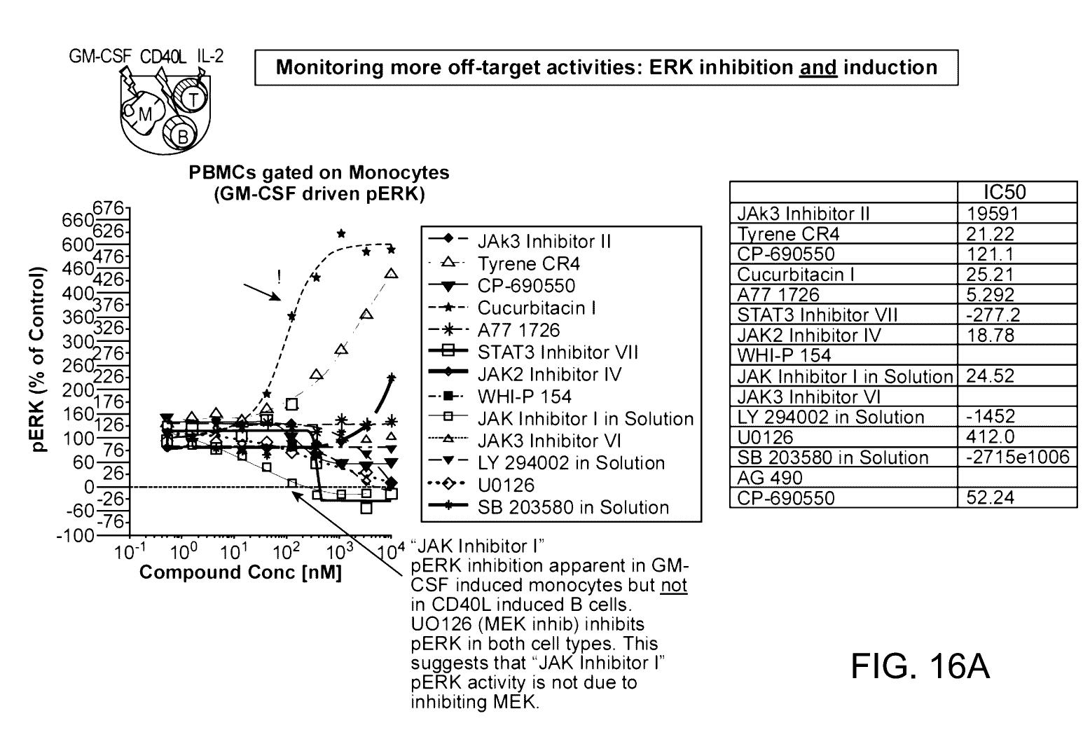

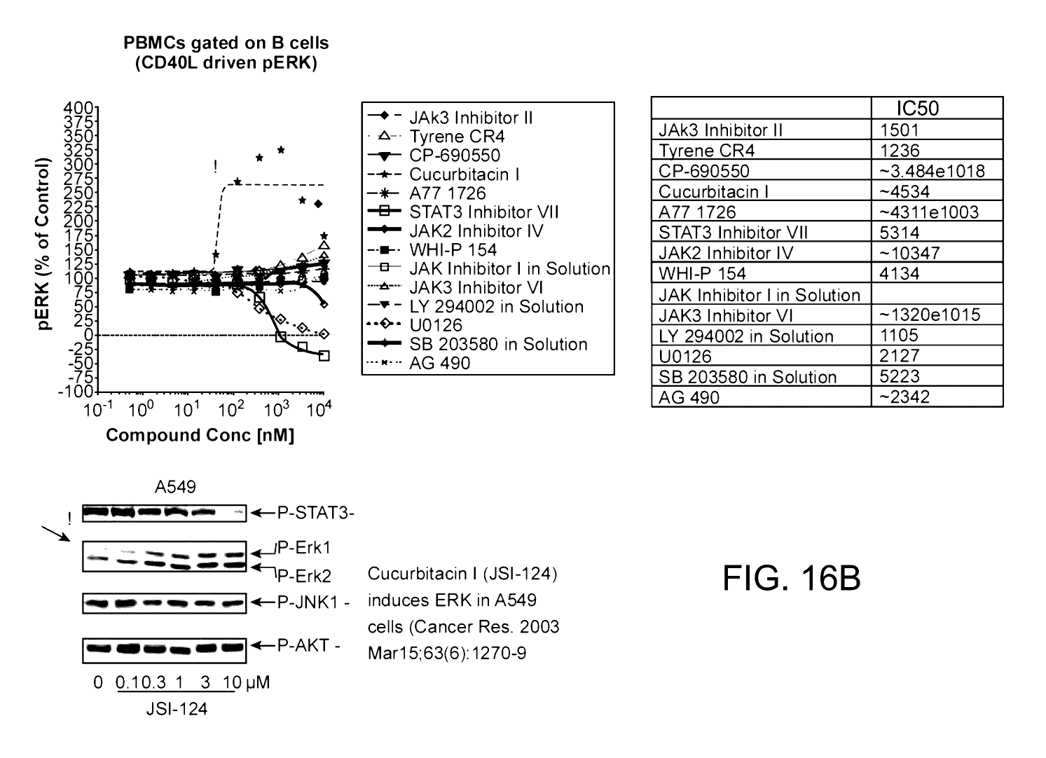

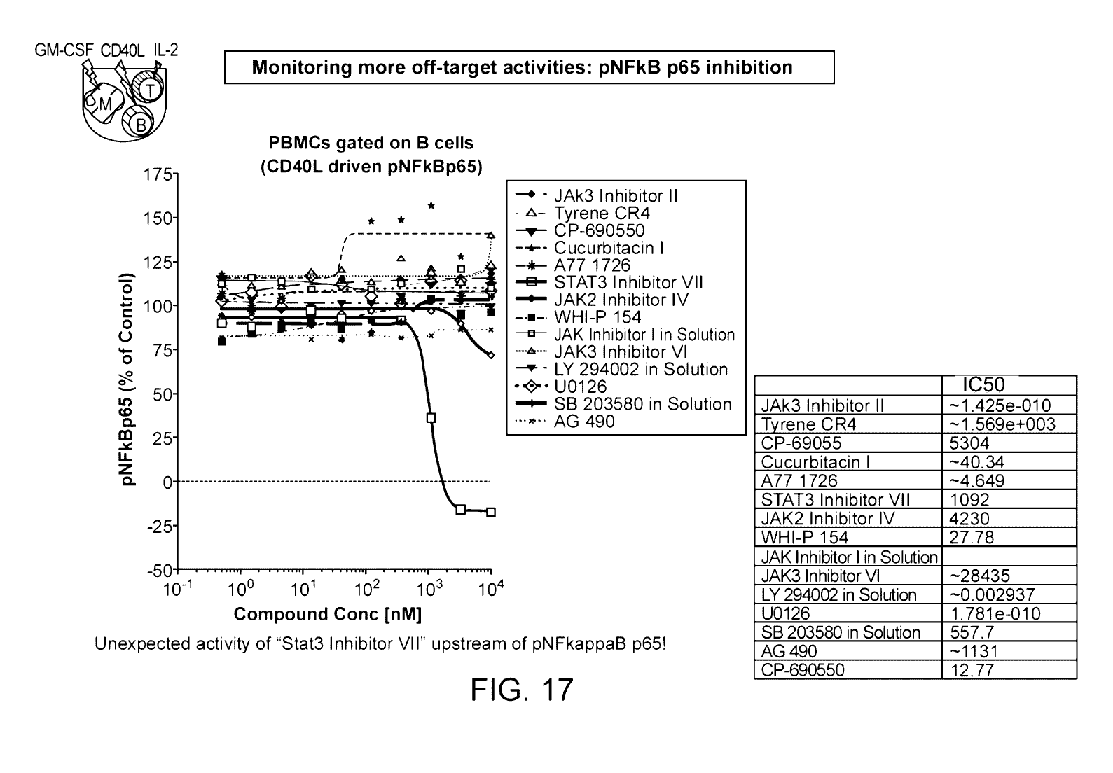

In some embodiments of the invention, phospho-flow it used to perform dose-response experiments with potential therapeutics in a complex tissue such as whole peripheral blood. Multiparameter phospho-flow analysis permits evaluation of the effects of a JAK/STAT inhibitor on cell sub-populations present in whole peripheral blood such as T cells, B-cells, non-T/non-B cells, monocytes as well as other rare cell sub-populations, such as CD34+ hematopoietic progenitor cells. The ability to assay the outside and inside of a cell simultaneously bypasses the need to isolate the individual cell types, some of which are rare (for example: CD34+CD38-hematopoietic progenitors). In contrast to some of the most advanced cell-based screens where it can be difficult to assay target inhibition across different cell subpopulations present in a heterogeneous sample, multiparameter phospho-flow cytometry enables the measurement of cell type selectivity of a compound for the same target by the use of markers which are used to delineate different cell types. The concurrent use of phospho-specific antibodies measures target inhibition in each cell sub-population. An example is shown in FIG. 8, in which the specificity of a JAK3 inhibitor is confirmed in T-cells stimulated by IL-2. Dosing experiments such as the ones depicted in FIG. 8 may be used to identify the potency of different inhibitor compounds against the JAK/STAT pathway. There is marginal inhibition of GM-CSF-mediated JAK2 activity in neutrophils. p-STAT5 is the signaling molecule readout for the amount of JAK inhibition in both cell sub-sets. Thus, the activation of STAT5 is mechanistically different in a T-cell versus a neutrophil. The methods of the invention may also identify off-target effects of potential therapeutics on other signaling pathways. An example is shown in FIGS. 16-17, in which multiparameter phosphoflow identifies off-target effects of JAKISTAT inhibitors on the ERKJMAPK and NFkB pathways, which are given by way of example only.

Flow cytometry experiments ideally include a reference sample against which experimental samples are compared. Reference samples can include normal and/or cells associated with a condition (e.g. tumor cells). Reference samples can also comprise subpopulations of cells in the same patient sample. See also U.S. patent application Ser. No. 12/501,295 for visualization tools.

The patients are stratified based on nodes that inform the clinical question using a variety of metrics. To stratify the patients between those patients with No Response (NR) versus a Complete Response (CR), a prioritization of the nodes can be made according to statistical significance (such as p-value or area under the curve) or their biological relevance.

Four metrics may be used to analyze data from cells that may be subject to a disease, such as AML. For example, the “basal” metric is calculated by measuring the autofluorescence of a cell that has not been stimulated with a modulator or stained with a labeled antibody. The “total phospho” metric is calculated by measuring the autofluorescence of a cell that has been stimulated with a modulator and stained with a labeled antibody. The “fold change” metric is the measurement of the total phospho metric divided by the basal metric. The quadrant frequency metric is the frequency of cells in each quadrant of the contour plot.

A user may also analyze multimodal distributions to separate cell populations. A user can create other metrics for measuring the absence of signal, or a negative control. For example, a user may analyze autofluorescence in a “gated unstained” or ungated unstained population as the negative signal for calculations such as “basal” and “total”. This is a population that has been labeled with surface markers such as CD33 and CD45 to gate the desired population, but is unstained for with the fluorescent reagents that will be used for quantitatively determining node states. However, every antibody has some degree of nonspecific binding activity or “stickyness” which is not taken into account by measuring only autofluorescence of untreated cells. In one embodiment, the user may contact cells with one or more isotype-matched antibody to assess non-specific binding. In one embodiment, the antibodies are contacted with peptides or phosphopeptides with which the antibody should bind. This treatment may inhibit an antibody's epitope-specific binding activity by blocking its antigen binding site. Consequently, contacting cells with the “bound” antibody may allow measurements of non-specific binding. In another embodiment, a user may measure nonspecific binding by blocking specific epitopes with an unlabeled clone or clones of the antibody or antibodies of interest, and then contacting cells with the antibody of interest. In another embodiment, a user may block using other solutions with high protein concentrations including, but not limited to fetal bovine serum, and normal serum of the species in which the antibodies were made (e.g. using normal mouse serum to block before treatment with a mouse antibody). Label-conjugated primary antibodies are preferred over unlabeled primary antibodies detected by label-conjugated secondary because the secondary antibodies will recognize the blocking serum. In another embodiment, a user may identify nonspecific binding by treating fixed cells with phosphatases to remove phosphate groups, and then contact the cells with antibodies directed at the phophorylated epitopes.

In alternative embodiments, other methods of data analysis may be used, for example third color analysis (3D plots), which can be similar to Cytobank 2D, plus third D in color.

### Kits

In some embodiments the invention provides kits. Kits provided by the invention may comprise one or more of the state-specific binding elements described herein, such as phospho-specific antibodies. A kit may also include other reagents that are useful in the invention, such as modulators, fixatives, containers, plates, buffers, therapeutic agents, instructions, and the like. See U.S. Ser. No. 61/245,000.

In some embodiments, the kit comprises one or more of the phospho-specific antibodies specific for the proteins selected from the group consisting of PI3-Kinase (p85, p110a, p110b, p110d), Jak1, Jak2, Lnk, SOCS3, SH2-B, Rac, Rho, Cdc42, Ras-GAP, Vav, Tiam, Sos, Dbl, Nck, Gab, PRK, SHP1, and SHP2, SHIP1, SHIP2, sSHIP, PTEN, She, Grb2, PDK1, SGK, Akt1, Akt2, Akt3, TSC1,2, Rheb, mTor, 4EBP-1, p70S6Kinase, S6, LKB-1, AMPK, PFK, Acetyl-CoAa Carboxylase, DokS, Rafs, Mos, Tp12, MEK1/2, MLK3, TAK, DLK, MKK3/6, MEKK1,4, MLK3, ASK1, MKK4/7, SAPK/JNK1,2,3, p38s, Erk1/2, Syk, Btk, BLNK, LAT, ZAP70, Lck, Cbl, SLP-76, PLC_{γ1}, PLC_{γ2}, STAT1, STAT 3, STAT 4, STAT 5, STAT 6, FAK, p130CAS, PAKs, LIMK1/2, Hsp90, Hsp70, Hsp27, SMADs, Rel-A (p65-NFKB), CREB, Histone H2B, HATs, HDACs, PKR, Rb, Cyclin D, Cyclin E, Cyclin A, Cyclin B, P16, pl4Arf, p27KIP, p21CIP, Cdk4, Cdk6, Cdk7, Cdk1, Cdk2, Cdk9, Cdc25,A/B/C, Abl, E2F, FADD, TRADD, TRAF2, RIP, Myd88, BAD, Bcl-2, Mcl-1, Bel-XL, Caspase 2, Caspase 3, Caspase 6, Caspase 7, Caspase 8, Caspase 9, IAPs, Smac, Fodrin, Actin, Src, Lyn, Fyn, Lck, NIK, IκB, p65(Re1A), IKKα, PKA, PKCα, PKCβ, PKCθ, PKCδ, CAMK, Elk, AFT, Myc, Egr-1, NFAT, ATF-2, Mdm2, p53, DNA-PK, Chk1, Chk2, ATM, ATR, β-catenin, CrkL, GSK3α, GSK3β, and FOXO. In some embodiments, the kit comprises one or more of the phospho-specific antibodies specific for the proteins selected from the group consisting of Erk, Syk, Zap70, Lck, Btk, BLNK, Cbl, PLCγ2, Akt, Re1A, p38, S6. In some embodiments, the kit comprises one or more of the phospho-specific antibodies specific for the proteins selected from the group consisting of Akt1, Akt2, Akt3, SAPKANK1,2,3, p38s, Erk1/2, Syk, ZAP70, Btk, BLNK, Lck, PLCγ PLCγ2, STAT1, STAT 3, STAT 4, STAT 5, STAT 6, CREB, Lyn, p-S6, Cbl, NF-κB, GSKβ, CARMA/Bcl10 and Tcl-1.

In some embodiments, the kit comprises one or more antibodies that recognize non-phospho and phospho epitopes within a protein, including, but not limited to Lnk, SOCS3, SH2-B, Mpl, Epo receptor, and Flt-3 receptor. Kits may also include instructions for use and software to plan, track experiments, and files which contain information to help run experiments.

Kits provided by the invention may comprise one or more of the modulators described herein.

The state-specific binding element of the invention can be conjugated to a solid support and to detectable groups directly or indirectly. The reagents may also include ancillary agents such as buffering agents and stabilizing agents, e.g., polysaccharides and the like. The kit may further include, where necessary, other members of the signal-producing system of which system the detectable group is a member (e.g., enzyme substrates), agents for reducing background interference in a test, control reagents, apparatus for conducting a test, and the like. The kit may be packaged in any suitable manner, typically with all elements in a single container along with a sheet of printed instructions for carrying out the test.

Such kits enable the detection of activatable elements by sensitive cellular assay methods, such as IHC and flow cytometry, which are suitable for the clinical detection, prognosis, and screening of cells and tissue from patients, such as leukemia patients, having a disease involving altered pathway signaling.

Such kits may additionally comprise one or more therapeutic agents. The kit may further comprise a software package for data analysis of the physiological status, which may include reference profiles for comparison with the test profile.

Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. Kits described herein can be provided, marketed and/or promoted to health providers, including physicians, nurses, pharmacists, formulary officials, and the like. Kits may also, in some embodiments, be marketed directly to the consumer. Components shown in the examples below may be included in kits of the present invention.

One embodiment of the present invention is a reproducible assay that evaluates the in vitro potency and selectivity of commercial and investigational JAK/STAT inhibitors in primary cells from healthy individuals. Peripheral blood and bone marrow samples will be treated in vitro with inhibitor alone or in combination with relevant modulators of the JAK/STAT and parallel pathways. These studies will characterize inhibition of multiple components of the JAK/STAT pathway simultaneously in single cells while at the same time characterizing whether the inhibitors have activity against other parallel intracellular pathways. These foundational experiments in samples from healthy individuals will generate a reference dataset against which subsequent analysis of samples acquired from patients with hematological malignancies can be compared. Specifically hematological malignancies will be chosen in which members of the JAK family are activated.

Another embodiment of the present invention is evaluating the potency and selectivity of commercial and investigational JAK/STAT inhibitors on primary samples acquired from patients diagnosed with hematologic malignancies. Specifically in myeloproliferative neoplasms the JAK/STAT pathway is activated either through gain of function mutations in JAK, or in receptors that confer potentiation of JAK activity. Additionally, in a diverse number of hematological malignancies, JAK activity may be increased through chromosomal translocations in which its C-terminal kinase domain is fused with pericentriolar material (PCM1) or with TEL. Other mechanisms by which the JAK/STAT pathway may be activated are through cytokine receptors such as G-CSF and GM-CSF noted for their activity in, for example, Acute Myeloid Leukemia (AML) and Juvenile Myelomonocytic Leukemia (JMML) respectively. The potency and selectivity determined for the JAK/STAT inhibitors in cell sub-sets within samples from healthy individuals will be compared with the potency and selectivity determined for the same pathway parameters in samples taken from diseased patients.

Another embodiment of the present invention is to utilize the potency and efficacy assays to evaluate the effects of JAK/STAT inhibitors on signaling in rare hematopoietic cell populations, including stem cells, afforded by the ability of the technology to analyze limited numbers of cells. Potency and selectivity profiles of JAK/STAT inhibitors may be derived for their targets/pathways in these rare cell populations.

### Drug Dosing, Potency, and Specificity

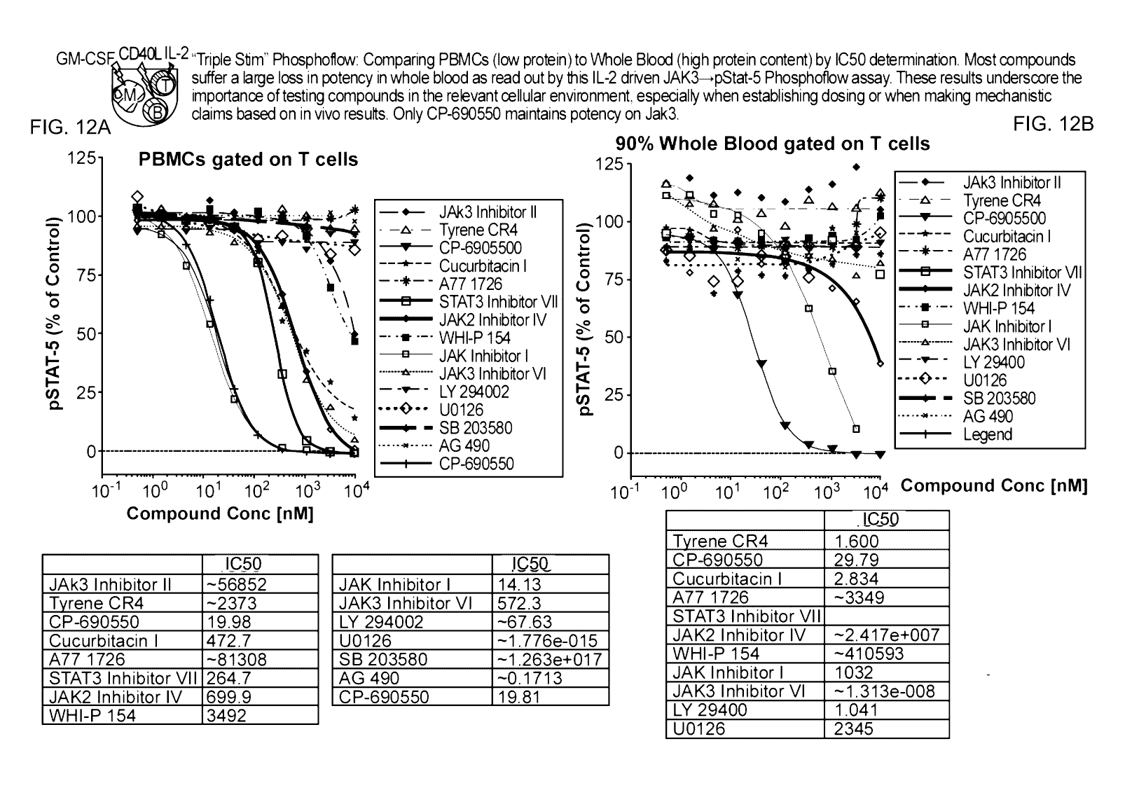

In some embodiments, the invention can be used to measure drug potency and specificity in a single assay using physiologically relevant samples. The efficacy of a drug compound might vary by patient and cell type, depending, for example, on physiological, genetic, and epigenetic differences between patients, or between cells types. The invention provides methods for measuring the potency and selectivity of a drug or combination of drugs for a target cell type and pathways as well as its effects on undesired (off-target) cell types and pathways. A patient sample without the need to sort cell types, for example whole blood, may be treated with 1, 2, 3, 4, 5, or more modulators that stimulate cell signaling in combination with 1, 2, 3, 4, 5 or more drug compounds. The modulators may stimulate signaling in one or more cell types. For example a combination of GM-CSF, CD4OL, and IL-2 (“Triple stim”) may be used to stimulate multiple pathways in Monocytes, B cells, and T cells simultaneously (see FIG. 12). Drug dosing may be the same or different for each drug compound, ranging from 1×10⁰ nM, 1×10¹ nM, 1×10² nM, 1×10³ nM, 1×10⁴ nM or greater. Treatment scheduling may be the same or different for each drug compound, and may comprise continuous treatment or alternating of intervals of treatment and non-treatment. Each treatment (or interval) may range from 1, 2, 3, 4, 5, 6, 7, 8, 9, 10 or more minutes up to an hour or fraction thereof, 1, 2, 3, 4, 5, 6, 7, 8, 9, 10, hours plus a fraction thereof, up to one day, and 1, 2, 3, 4, 5, 6, 7 or more days plus a fraction thereof. Single cell signaling activity can be measured in fixed and permeablized cells using fluorescently-labeled antibodies that detect changes in the states of activatable elements in signaling pathways, including phosphorylation, acetylation, methylation, ubiquitination, sumoylation, protein modifications, conformational changes, and cleavage of proteins in a signaling pathway, for example the JAK/STAT, ERK, and NFkB pathways. As will be appreciated by one skilled in the art, this method can be applied to any cell signaling pathway or combination of signaling pathways.

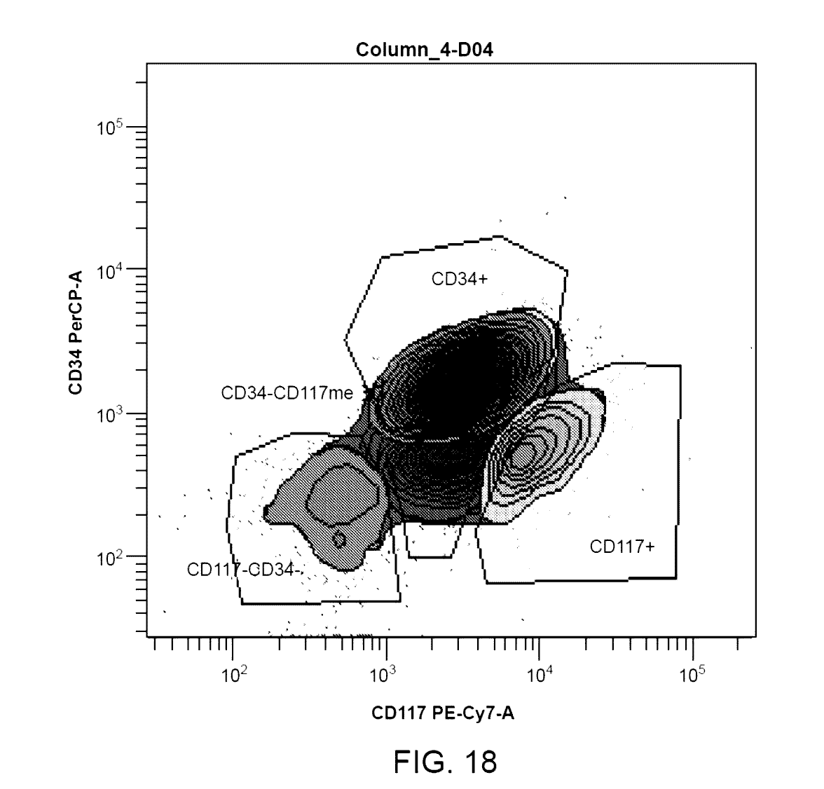

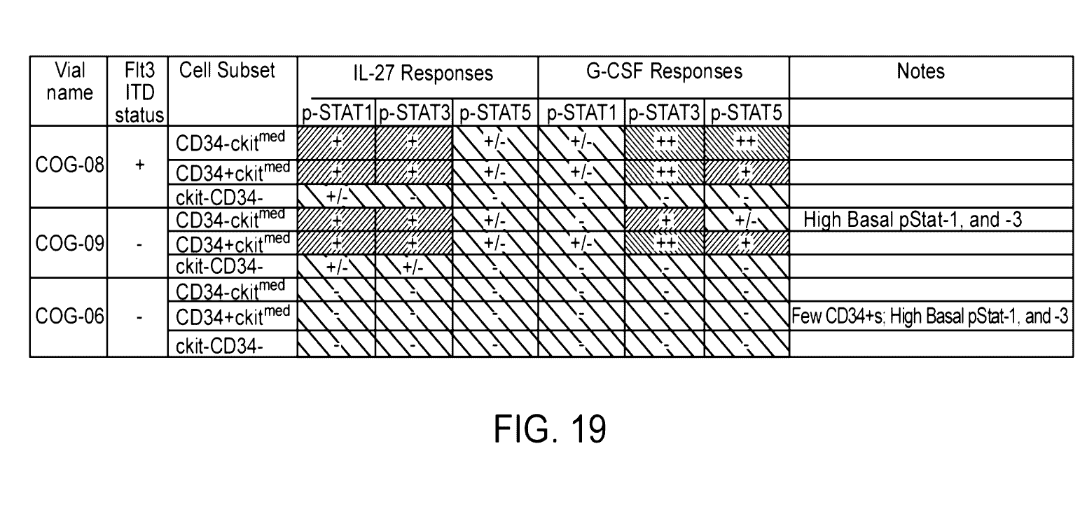

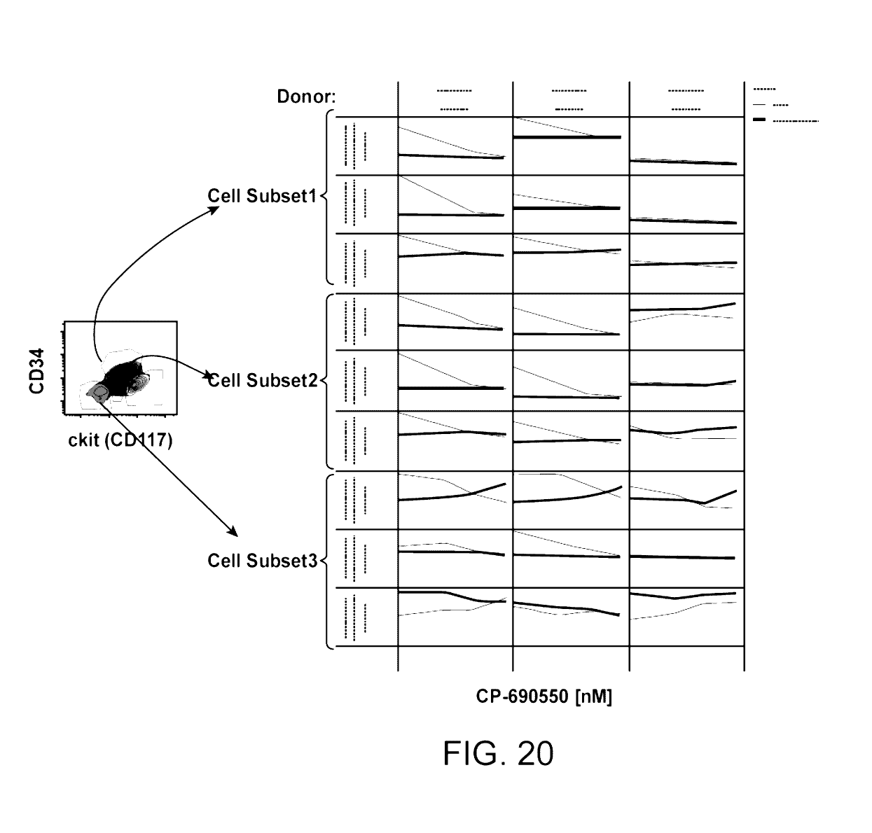

Following treatment with modulators and a drug or combination of drugs, multiparametric flow cytometry can be used to measure activity levels of multiple signaling pathways in multiple cell populations within the same assay (See, for example, FIGS. 19-20, showing the measurement of p-STAT5 and p-ERK levels in Monocytes, B cells and T cells within the same sample). Additionally, multiparametric flow cytometry can measure the activity of family members of the same signaling pathway (See, for example, FIGS. 18-19, comparing Jak3-driven p-STAT levels in T cells to Jak-2 driven p-STAT5 levels in Monocytes). Drug dose titration based on single cell signaling activity can be used to generate a drug dose response curve and calculate the potency and selectivity of a drug for specific cell types and specific signaling pathways (See FIG. 14). This method can be used to identify dose-response for targeted cell types and signaling pathways as well as undesired (off-target) cell types and signaling pathways (See FIGS. 15-17, assaying the effects of compounds on Jak2, Jak3, ERK, and NFkB signaling). A clinically useful drug dose must impact the target, and therefore can be no lower than the minimum dose that substantially affects activity of a target pathway in a specific cell type. At the same time, a clinically useful dose should minimize undesired off-target toxicity, and therefore should be no higher than the minimum dose that that substantially affects signaling activity in off-target pathways or off-target cell types. For example, FIG. 14 illustrates methods of the invention that use a whole blood sample to select a dosing regimen for CP-6905550, a JAK3 inhibitor compound in T cells: the dose must be above the IC50 needed to inhibit JAK3 signaling in T cells, but below the IC50 at which the drug begins to inhibit JAK2 signaling in monocytes. One skilled in art will appreciate that the methods of the invention can be applied generally to calculate a clinical drug dose by identifying a dose range wherein specific target activity is achieved, while minimizing undesired side effects.

In some embodiments, the methods of the invention can be used for screening drug compounds and determining their mechanism of action, for example by inferring their effects on signaling pathways. In some embodiments, the methods of the invention can be used for calculating dose and scheduling of a drug compound or combination of compounds in preclinical studies. In some embodiments, the methods of the invention can be used for determining target drug doses in phase 1 and phase 2 clinical trials. Since the methods of the invention can be used to identify drug effects in whole blood samples, these effects are likely to predict the effects of the drug when administered to the patient who donated the sample. Therefore, the methods of the invention can also be used at the level of the individual patient, including the selection of a drug or a combination of drug, drug scheduling, and monitoring the development of drug resistance in patients. Although the preferred embodiment of the invention uses whole blood samples or other physiologically relevant hematopoetically-derived cell samples, in some embodiments, the methods of the invention can be used on other tissues. For example, if signaling pathways in subsets of whole blood cells are identified as surrogates for signaling pathways in other tissues, whole blood samples may be used as a model to assess drug effect in these other tissues. Alternatively, protocols for dissociating cells from solid tissues, for example tumors, may allow cells from these tissues to be assayed using the methods of the invention.

### EXAMPLES

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are expressly incorporated by reference in their entireties.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1

The present illustrative example represents how to analyze cells in one embodiment of the present invention. There are several steps in the process, such as the stimulation step, the staining step and the flow cytometry step. The stimulation step of the phospho-flow procedure can start with vials of frozen cells and end with cells fixed and permeabilized in methanol. Then the cells can be stained with an antibody directed to a particular protein of interest and then analyzed using a flow cytometer. A protocol similar to the following was used to analyze AML cells from patient samples.

Materials:
Compound (See Table 8 for a list of compounds that may be used)
DMSO
Thawing media: PBS-CMF+10% FBS+2 mM EDTA
70 um Cell Strainer (BD)
Anti-CD45 Alexa 700 (Invitrogen)—Use 1 ul per sample.
Propidium Iodide (PI) Solution (Sigma 10 ml, 1 mg/ml)—Use at 1 ug/ml.
RPMI+1% FBS
Media A: RPMI+1% FBS+1× Penn/Strep
Live/Dead Reagent, Amine Aqua (Invitrogen)
2 ml, 96-Deep Well, U-bottom polypropylene plates (Nunc)
300 ul 96-Channel Extended-Length D.A.R.T. tips for Hydra (Matrix)
Phosphate Buffered Saline (PBS) (MediaTech)
16% Paraformaldehyde (Electron Microscopy Sciences)
100% Methanol (EMD) stored at −20 C.
Transtar 96 dispensing apparatus (Costar)
Transtar 96 Disposable Cartridges (Costar, Polystyrene, Sterile)
Transtar reservoir (Costar)
Foil plate sealers

Thawing cell and live/dead staining:
1) Thaw frozen cells in a 37° C. water bath. Gently resuspend the cells in the vial and transfer to the 15 mL conical tube. Centrifuge the 15 mL tube at 930 RPM (200×g) for 8 minutes at room temp. Aspirate the supernatant and gently resuspend the pellet in 1 mL media A. Filter the cell suspension through a 70 um cell strainer into a new 15 mL tube. Rinse the cell strainer with 1 mL media A and another 12 ml of media A into the 15 mL tube. Mix the cells into an even suspension. Immediately remove a 20 iL aliquot into a 96-well plate containing 180 μL PBS+4% FBS+CD45 Alexa 700+PI to determine cell count and viability post spin. After the determination, centrifuge the 15 mL tubes at 930 RPM (200×g) for 8 minutes at room temp. Aspirate the supernatant and gently resuspend the cell pellet in 4 mL PBS+4 μL Amine Aqua and incubate for 15 min in a 37° C. incubator. Add 10 mL RPMI+1% FBS and invert the tube to mix the cells. Centrifuge the 15 mL tubes at 930 RPM (200×g) for 8 minutes at room temp. Resuspend the cells in Media A at the desired cell concentration (1.25×10⁶/mL).
a. For samples with low numbers of cells (<18.5×10⁶), resuspend in up to 15 mL media.
b. For Samples with high numbers of cells (>18.5×10⁶), raise the volume to 10 mL with media A and transfer the desired volume to a new 15 mL tube, adjusting the cell concentration to 1.25×10⁶ cells/mL Transfer 1.6 mL of the above cell suspension (concentration is at 1.25×10⁶ cells/mL, into wells of a multi-well plate. From this plate, distribute 80 ul into each well of a subsequent plate. Cover plates with a lid (Nunc) and place in 37° C. incubator for 2 hours to rest.

Compound Screening:

Prepare serial dilutions of test compound to reach a final desired concentration, then incubate cells with compound for 1 hour at 37° C.

Cell Stimulation
1) Prepare a concentration for each stimulant that is five-fold more (5×) than the final concentration using Media A as diluent Array 5× stims in a standard 96 well v-bottom plate that correspond to the wells on plate with cells to be stimulated.
2) Preparation of fixative: Stock vial contains 16% paraformaldehyde which is diluted with PBS to a concentration that is 1.5×. Place in 37° C. water bath.
3) Adding the stimulant: Take the cell plate(s) out of the incubator and place in a 37° C. water bath. Take cell plate from water bath and gently swirl plate to resuspend any settled cells. With pipettor, dispense the stimulant into the cell plate and hold over vortex set to “7” and mix for 5 sec. Place deep well plate back into the water bath.
4) Adding Fixative: Dispense 200 μl of the fixative solution (final concentration is 1.6%) into wells and then mix on the titer plate shaker on high for 5 sec. Cover plate with foil sealer and float in 37° C. water bath for 10 min. Spin plate (6 min 2000 rpm, room temp). Aspirate cells using a 96 well plate aspirator (VP Scientific). Vortex plate to resuspend cell pellets in the residual volume. Ensure the pellet is dispersed before the Methanol step (see cell permeabilization) or clumping will occur.
5) Cell Permeabilization: Add permeability agent (which can be but is not limited to methanol) slowly and while the plate is vortexing. To do this, place the cell plate on titer plate shaker and make sure it is secure. Set the plate to shake using the highest setting. Use a pipetter to add 0.6 mls of 100% methanol to plate wells. Place plate(s) on ice until this step has been completed for all plates. Cover plates with a foil seal using the plate roller to achieve a tight fit. At this stage the plates may be stored at −80° C.

Staining Reagents
1) FACS/Stain Buffer-PBS+0.1% Bovine serum albumen (BSA)+0.05% Sodium Azide.
2) Diluted Bead Mix-1 mL FACS buffer+1 drop anti-mouse Ig Beads+1 drop negative control beads.

Staining Protocol
1) Thaw cells if frozen.
2) Pellet cells at 2000 rpm 5 minutes.
3) Aspirate supernatant with vacuum aspirator.
4) Vortex on the “Plate Vortex” for 5-10 sec.
5) Wash cells with 1 mL FACS buffer.
6) Spin, Aspirate and Vortex as above.
7) Add 50 μL of FACS/stain buffer with the desired, previously optimized, antibody cocktail to 2 rows of cells at a time and agitate.
8) Cover and incubate on plate shaker for 30′ at Room Temp (RT).
9) During this incubation, prepare the compensation plate.
   a. In a standard 96 well V-bottom plate, add 20 μL of “diluted bead mix” per well.
   b. Each well gets 5 μL of 1 fluorophor conjugated control IgG (examples: Alexa488, PE, Pac Blue, Aqua, Alexa647, Alexa700). For the Aqua well, add 200 uL of Aqua−/+ cells.
   c. Incubate 10 minutes RT.
   d. Wash by adding 200 μl FACS/stain buffer, centrifuge at 2000 rpm for 5 minutes, and remove supernatant.
   e. Repeat step d, resuspend in 200 μL FACS/stain buffer and transfer to U-bottom 96 well plate.
10) After 30 min, add 1 mL FACS/stain buffer and incubate plate on plate shaker for 5 minutes at room temperature.
11) Centrifuge, aspirate and Vortex cells as above. Add 1 mL FACS/stain buffer, cover & incubate on plate shaker for 5 minutes at room temperature.
12) Repeat 11) and 12) but resuspend in 75 μl FACS/stain buffer.
13) Analyze the cells using a flow cytometer, such as a LSRII (Becton Disckinson), select all wells and set Loader Settings
   a. Flow Rate: 2 uL/sec
   b. Sample Volume: 40 uL
   c. Mix volume: 40 uL
   d. Mixing Speed: 250 uL/sec
   e. # Mixes: 5
   f. Wash Volume: 800 uL
   g. Standard 96 well plate mode
14) When plate has completed, perform a Batch Analysis to ensure no clogs.

Gating Protocol Take the data acquired from the flow cytometer and analyze with Flowjo software (Treestar, Inc). The Flow cytometry data is first gated on single cells (to exclude doublets) using Forward Scatter Characteristics Area and Height (FSC-A, FSC-H). Single cells are gated on live cells by excluding dead cells that stain positive with an amine reactive viability dye (Aqua-Invitrogen). Live, single cells are then gated for subpopulations using antibodies that recognize surface markers as follows: CD45++, CD33− for lymphocytes, CD45++, CD33++ for monocytes+granulocytes and CD45+, CD33+ for leukemic blasts. Signaling, determined by the antibodies that interact with intracellular signaling molecules, in these subpopulation gates that select for “lymphs”, “monos+grans, and “blasts” is analyzed. Inclusion of other antibodies to cell surface markers can be incorporated to further define the cell subpopulations, including the following: CD19+ or CD20+ for B cells; CD3+ for T cells, CD56+ for NK cells; CD14+ for monocytes, CD34+ for progenitor cells.

The data can then be analyzed using various metrics, such as basal level of a protein or the basal level of phosphorylation in the absence of a stimulant, total phosphorylated protein, or fold change (by comparing the change in phosphorylation in the absence of a stimulant to the level of phosphorylation seen after treatment with a stimulant), on each of the cell populations that are defined by the gates in one or more dimensions. These metrics are then organized in a database tagged by: the Donor ID, plate identification (ID), well ID, gated population, stain, and modulator. These metrics tabulated from the database are then combined with the clinical data to identify nodes that are correlated with a pre-specified clinical variable (for example; response or non response to therapy) of interest.

### Example 2

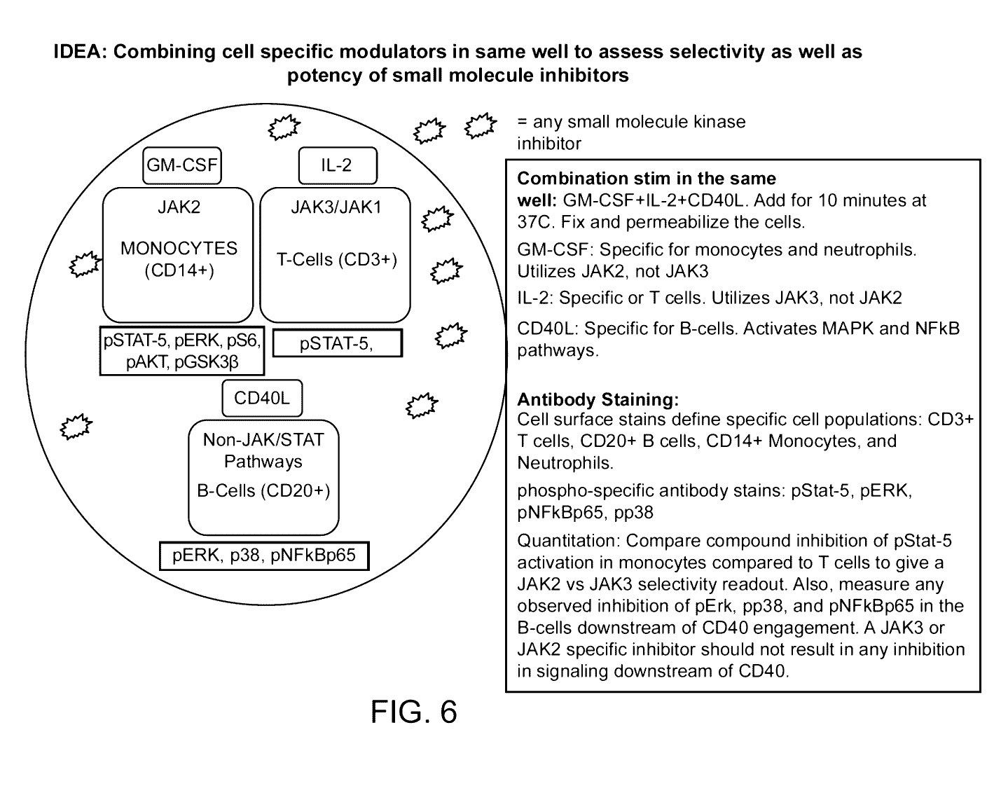

Described below is an assay to determine selectivity and potency of test compounds including but not limited to, small molecule kinase inhibitors. The assay would simultaneously measure, in one or more tubes or wells, the selectivity of an inhibitor for its inhibition of JAK2 vs JAK3. The same assay, would also measure any inhibitory activity of the small molecule kinase inhibitor for signaling molecules within the Ras-Raf-Erk pathway, the NFκB pathway, and the p38 pathway. See FIG. 6 for a proposed test.

The small molecule kinase inhibitor(s) of interest would be incubated with whole blood, peripheral blood mononuclear cells (PBMCs), or bone marrow for 1 hour. A combination of cell signaling agonists consisting of GM-CSF, IL-2 and CD40L would be added to the cells for 10 minutes at 37° C. The phospho-flow fix and permeabilization protocol shown in the above examples would then be added to the cells. Incubation with fluorochrome-conjugated antibodies that recognize peptide epitopes within phenotypic markers expressed on cells would delineate cell sub-sets. Examples include, but are not limited to, CD14, CD20, and CD3 which would discriminate monocytes, B cells, and T cells respectively. A cocktail of phospho-specific antibodies directed to pStat-5, pErk, pNFκB (p65), and pp-38, all conjugated to distinct fluorophores would be included in the staining mixture. Flow cytometry would identify the discrete cell types. For each cell type, the fluorescence of the phospho-specific antibodies would be quantified by median or mean fluorescent intensity values.

Within the Jak family of intracellular signaling molecules, GM-CSF signals exclusively through Jak2 and activates Jak2 in cells that express the GM-CSF receptor including but not limited to monocytes and neutrophils. Activation of Jak2 in these cells, mediated by GM-CSF can be used to determine the potency of Jak2 inhibitors. Within the Jak family of intracellular signaling molecules, IL-2 signals through Jak1 and Jak3 and activates Jak1 and Jak3 in cells that express engages the IL-2 receptor including but not limited to T cells and NK cells. Activation of Jak3 and Jak1 in these cells mediated by IL-2 can be used to determine the potency of Jak1 and Jak3 inhibitors. Activation of the CD40 pathway by treatment of B cells with CD40 ligand results in increased signaling of several intracellular signaling pathways including but not limited to, the Ras-Raf-Erk pathway, the NFkB pathway and the p-38 pathway. Thus any inhibitor can be evaluated for its ability to inhibit CD40 mediated intracellular signaling pathways including but not limited to, the Ras-Raf-Erk pathway, the NFkB pathway and the p-38 pathway in B cells.

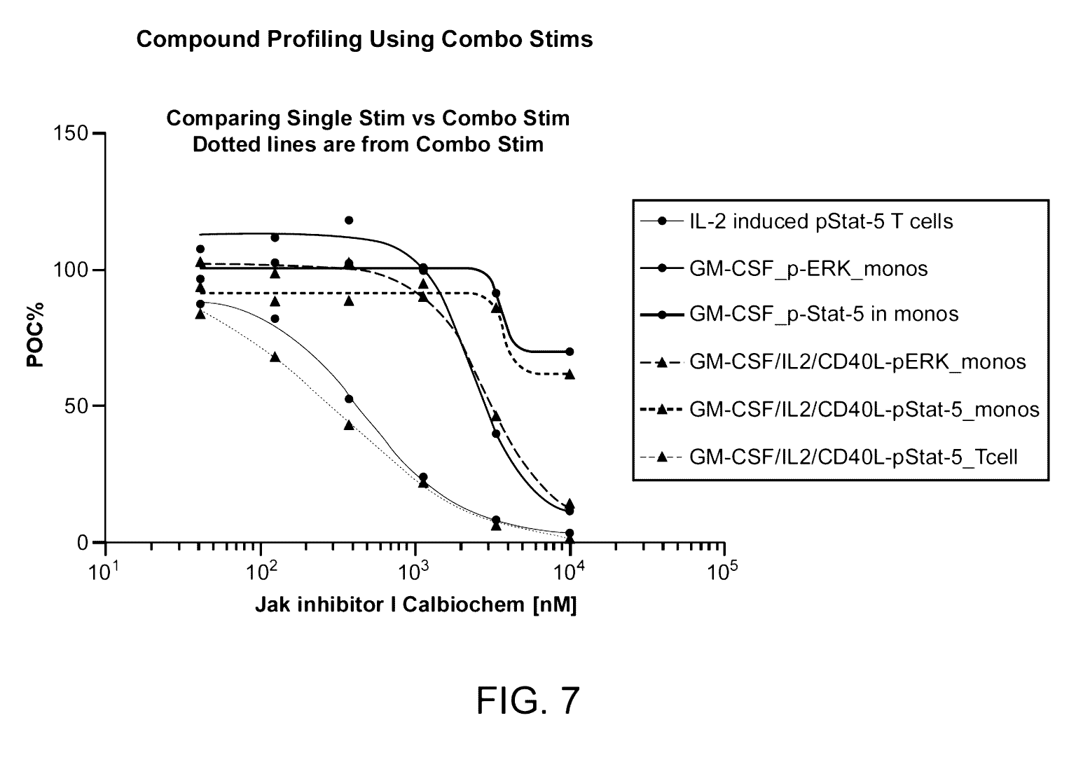

Overall this would be a useful assay to measure the potency of an inhibitor on multiple signaling pathways in multiple cell types downstream of cell specific modulators simultaneously within the same well that is used to perform the assay. See FIG. 7 which shows the proposed correspondence between the results in a single well versus multiple wells.

Other cell specific modulators can be combined into cocktails that provide activation of multiple signaling pathways in discrete cell types. Tables 1 thru 5 show cell specific modulators for classes of cells such as B cells, T cells, monocytes, CD34+ progenitors, and NK cells respectively. Various modulator cocktails can be created by choosing one or more modulators from two or more tables. The ability of a compound to modulate the activatable elements of the signaling cascades that are evoked from the particular modulator cocktail can be quantified via phosphoflow cytometry using a phospho-specific antibody specific to the element. The results would provide information on the selectivity and potency of the test compound in multiple cell types.

### Example 3

The following is an example of a method used to assay samples in some embodiments of the invention. It can be similar to the examples described above. Multiplex assays will be performed in a 96-well format. In brief, thawed or fresh samples will be incubated with varying concentrations of inhibitors for 1 hr at 37° C. followed by treatment with modulator (for example, IL-2, GM-CSF or IFNα) for 10 minutes. After, sample fixation and permeabilization, samples will be incubated with a cocktail of fluorochrome-conjugated antibodies designed to specify cell sub-sets including, but not limited to T-Lymphocytes, B-Lymphocytes, Monocytes, Myeloid cells, Myeloid Progenitors, Neutrophils, and all cells.

### Example 4

The following is an example using a method of the invention to screen the effects of different compounds—including JAK/STAT inhibitors—in human or mouse primary cells, which include whole blood, bone marrow, and splenocytes. Compounds selected from the list in Table 8 are tested in cell samples in 1% BSA that have been stimulated with three modulators: GM-CSF, CD-40L, and IL-2, which activate multiple signaling pathways in monocytes, B cells, and T cells, respectively. (See FIG. 11; Table 7). A dose series of treatments is performed for each compound, ranging from doses as low as no compound, up to doses in the ranges of 1×10° nM, 1×10¹ nM, 1×10² nM, 1×10³ nM, and 1×10⁴ nM. Cell signaling is measured by multiparametric phosphoflow cytometry to assess p-Stat3, pERK, and p-Stat5 levels. The samples are gated on cell populations. This method may be used, for example, to measure JAK/STAT signaling activity in gated T cells based on levels p-Stat5 (See FIG. 12). The relationship between dosing and signaling activity can be used to calculate the IC50 for each compound (See, e.g. FIG. 12). The methods of the invention can thus be used to assess the potency of different compounds and the specificity of these compounds. Consequently, the methods of the invention can be used to identify the effects of a modulator, such as a JAK/STAT inhibitor, on different signaling pathways in discrete cell populations to determine the specificity and potency of this compound. Additionally, these methods can be used to identify drugs that affect discrete cell types, and different signaling pathways.

In FIG. 12, a method of the invention demonstrates that the cellular environment strongly influences the potency of a modulator compound. In stimulated PBMCs, which have a relatively low concentration of extracellular protein, two compounds, CP-690550 and Pyridone 6 inhibit JAK/STAT signaling in gated T cells as measured by STAT5 phosphorylation, and have comparable potencies (IC50s). However, in 90% whole blood gated on T cells, which has a relatively high concentration of extracellular plasma proteins, CP-690550 retains a high potency, while the potency of Pyridone 6 is decreased 70-fold. Thus, in some embodiments, the invention can be used to assess the potency of a drug on a target cell population. The compounds from FIG. 12 are listed in Table 8.

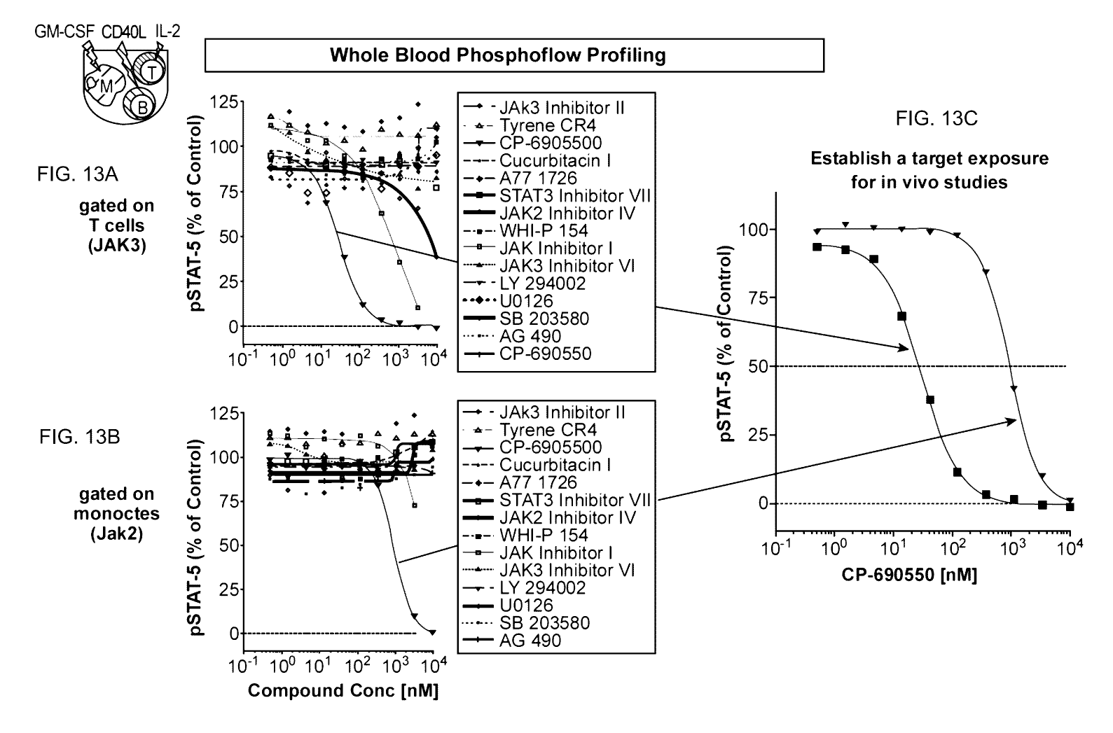

FIG. 13 shows that different JAK/STAT inhibitor compounds have different selectivities, depending on cell type. Jak2 is known to mediate signaling in monocytes downstream of GM-CSF stimulation. Jak3 is known to mediate signaling in lymphocytes downstream of IL-2 stimulation. The JAK kinase inhibitor compound CP-690550 preferentially inhibits Jak3. As shown in FIG. 13, analysis of p-STAT5 levels by flow cytometry demonstrates that CP-690550 has higher specificity for inhibiting Jak3 signaling in T-lympocytes than Jak2. Thus, in some embodiments, the methods of the invention can be used to assess the selectivity of a drug on a target population of cells. The compounds from FIG. 13 are listed in Table 8.

FIG. 15 shows that in some embodiments, the methods of the invention can measure the selectivity and potency of drug compounds in a single assay. Stimulated PBMCs are treated with the compounds in Table 8, and the IC50 of each compound is calculated for gated T cells and monocytes. Consistent with the separate findings that the compound CP-690550 has whole blood in vitro selectivity for Jak3 over Jak2, CP-690550's IC50 was 30-fold lower in T cells than in monocytes.

As shown in FIG. 14, the methods of the invention can be used for determining drug dose for patients. If a clinical dose is too low, a drug will have little effect, while if a dose is too high, a drug will have harmful side effects. For example, a pharmaceutically acceptable form of CP-690550 can be used to suppress a patient's immune system, but if the dose is too high, the pharmaceutically acceptable form of CP-690550 can also inhibit hematopoetic development, resulting in anemia, leucopenia, and thrombocytopenia. Thus, the optimal dose of a pharmaceutically acceptable form of CP-690550 in immunosuppressive therapy would be at least as high as the IC50 for T cells, but no higher than the IC50 for monocytes. Using these criteria, the methods of the invention would predict that the optimal dose of a pharmaceutically acceptable form of CP-690550 would be between 20 nM (T cell IC50) and 726 nM (monocyte IC50) (FIG. 15). As shown in FIG. 14, the target dose for CP-690550 of 160 nM would have been predicted as an optimal dose by the methods of the invention. See Changelian, P. S. et al (2003), Prevention of Organ Allograft Rejection by a Specific Janus Kinase 3 Inhibitor. Science 302: 875-78.

As shown in FIGS. 16-17, the methods of the invention can also be used to identify off-target effects of drug treatment. Muliparameter phosphoflow is used to detect the effects of compounds selected from the list in Table 8 on signaling pathways other than JAK/STAT. In FIG. 16, when PBMCs are treated with the JAK/STAT inhibitor Pyridone 6 (“Jak Inhibitor I,” Calbiochem), pERK levels are reduced in monocytes. However, Pyridone 6 does not reduce pERK levels in B cells. On the other hand, when PBMCs are treated with the STAT3 inhibitor cucurbitacin I, pERK is increased in both monocytes and B cells, demonstrating that cucurbitacin I has off-target effects as an activator of the ERK/MAPK pathway. Thus, the methods of the invention can identify both inhibition and induction of off-target signaling pathways, in this example, the ERK/MAPK pathway. The methods of the invention can also be used to identify off-target effects of JAK/STAT inhibitors on other pathways. FIG. 17 shows that multiparameter phosphoflow identifies that Stat3 Inhibitor VII inhibits NFkB signaling in stimulated B cells, as measured by levels of pNFkB65.

### Example 5

The following is an example using a method of the invention to screen the effects of a JAK/STAT inhibitor in cell samples from human patients with acute myeloid leukemia (AML). Cells from three patients were stimulated with the cytokines IL-27 and G-CSF to determine whether these modulators could induce JAK/STAT pathway activation across cells from different AML patient donors. IL-27 has been reported to signal through JAK1, JAK2, and Tyk2, leading to the phosphorylation of Stat1, Stat3, and Stat5. See Tables 6 and 7. G-CSF has been reported to signal through JAK2 and Tyk2 and leads to the phosphorylation of Stat3. See Tables 4, 6, 7, and 10. To compare inhibition of cytokine evoked JAK/STAT signaling in AML patient cells from the same three patients were then incubated with CP-690550, a JAK/STAT inhibitor listed on Table 8, at four concentrations (0 nM, 33 nM, 333 nM, and 3333 nM). One hour after incubation, the cells were stimulated with IL-27 and G-CSF. After stimulation cell signaling was measured by multiparametric phosphoflow cytometry to assess p-Stat1, p-Stat3 and p-Stat5 levels.

The samples were gated on cell populations. Incubation with fluorochrome-conjugated monoclonal antibodies that recognize lineage specific epitopes on the cell surface delineated at least 3 cell subpopulations in patient samples. FIG. 18 shows different cells populations based on basal expression of phenotypic surface markers such as CD34 and CD117. Three cell subsets were examined: (1) CD34−/CD117med, (2) CD34+/CD117med, (3) CD34−/CD117−. “CD117” in FIG. 18 is the same as “ckit” in FIG. 19. “Med” indicates a medium amount of expression with respect to other cell subsets that express more or less CD117. See FIGS. 18 and 19.

FIG. 19 shows heterogeneity in the response of patient cells to IL-27 and G-CSF stimulation. For example, donor TTM6034's cells showed no signaling while the other two donors show strong p-Stat1 responses to IL-27 stimulation. Cytokine responses were variable across donors and cell subsets.

IL-27 stimulation induced signaling in cells from two patient donors. See FIG. 19. When these cells were incubated with CP-690550 and then stimulated with IL-27, CP-690550 inhibited the p-Stat readout completely at the 333 nM concentration point. See FIG. 20. There was no inhibition of basal phosphorylation levels in the p-STAT readout. See FIG. 20.

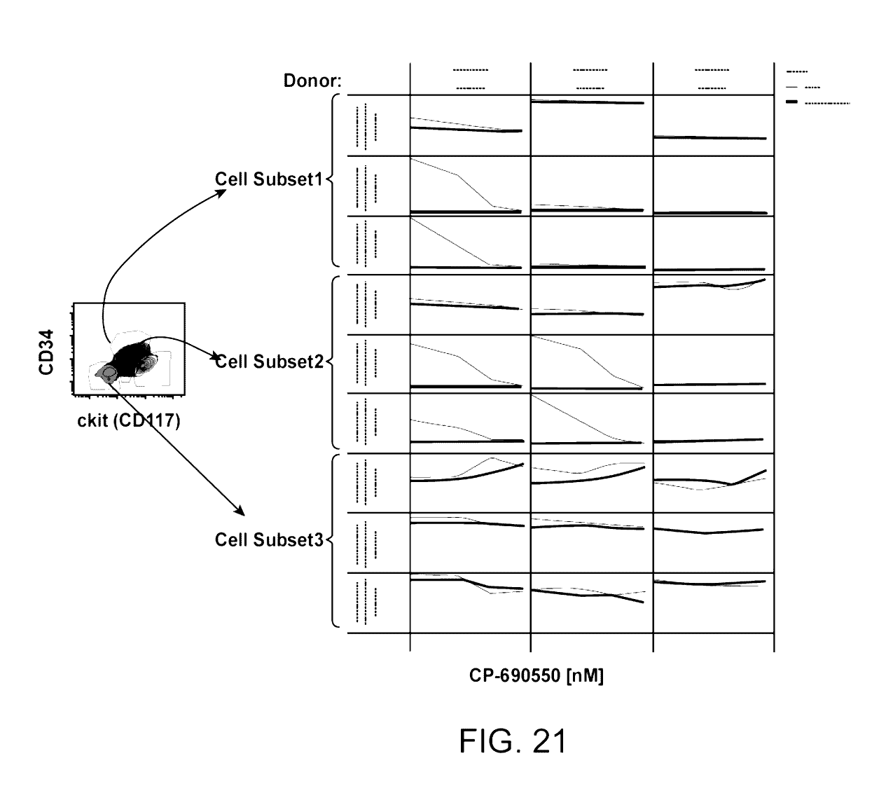

G-CSF stimulation induced signaling in cells from two patient donors. See FIG. 19. When these cells were incubated with CP-690550 and then stimulated with G-CSF, CP-690550 inhibited the p-Stat readout completely at the 3333 nM concentration point. See FIG. 20. As with cells stimulated with IL-27, there was no inhibition of basal phosphorylation levels in the p-Stat readout after CP-690550 incubation. See FIG. 21.

This Example shows that CP-690550 can inhibit IL-27 and G-CSF induced JAK/STAT signaling in AML patient bone marrow cells. The Example shows how the invention can be used to identify patients most likely to respond to an administered JAK/STAT inhibitor. CP-690550 inhibited the p-STAT readout at 333 nM (upon IL-27 stimulation) and 3333 nM (upon G-CSF stimulation) in cells from two of three patients. In cells from the third patient, however, IL-27 and G-CSF induced no signaling response and CP-690550 had no effect. The first two patients would be candidates for a CP-690550-based anti-cancer agent. The third would not.

TABLE 1

| CD20+ or CD19+ B cell | phospho specific antibodies appropriate |
|---|---|
| speific modulator | tor detection of activatable elements |
| Cross-linking the B cell | p-S6 Ribosomal Protein, p-Syk, |
| Receptor (BCR) with Anti-BCR | p-BLNK, pErk, p-Lck, pBtk, p-38, |
| antibodies (anti-IgM, | pAkt, p-NFkBp65 |
| IgG, IgD, IgE, IgA) | |
| CD4OL | pErk, p38, p-NFkBp65, p-S6 Ribosome, |
| | p-JNK |
| CpG oligonucliotides to | pErk, p-38, p-NFkBp65, p-MK2, p-JNK |
| stimulate through TLR9 | |
| receptors. | |
| Other B cell modulators: | pErk, p-38, pNFkBp-65 |
| BAFF, APRIL | |

TABLE 2

| CD3+ T cell specific | phosphor specific antibodies appropriate |
|---|---|
| modulators | for detection of activatable elements |
| Cross-linking the T cell | p-Zap70, pErk, p-Itk, p38, pAkt, |
| Receptor with antibodies | pNFkBp65, pJnk, p-S6 Ribosomal |
| to CD3 alone or combined | Protein, |
| with CD28 | |
| IL-2 | p-Stat-5 |
| IL-7 | p-STAT-5 |

TABLE 3

| CD33+ or CD14+ Monocyte | Anti-phospho specific antibodies |
|---|---|
| specific stimuli: | approriate for stimulation |
| GM-CSF | p-Akt, p-Erk, p-Itk, p-Stat-5, |
| | p-Stat3, p-S6 Ribosomal Protein, |
| LPS | p-Erk, p-38, pNFkBp65, pS6 Ribosome, |
| | p-MK2, HSP27, p-Jnk |
| Anisomycin | p-ERK, pp-38, p-NFkBp65, p-MK2 |
| Tumor Necrosis Factor | pERK, pp38, pNFkBp65, pMK2 |
| (TNF alpha) | |
| M-CSF | pAkt, p-Erk, p-PLCg, pS6 Ribosome |

TABLE 4

| CD34+ progenitor cell | phospho specific antibodies for s |
|---|---|
| specific stimuli: | detection of activatable elements |
| Erythropoietin | pStat-5, pErk, pS6 Ribosome |
| Thrombopoietin | pStat-5, pERK |
| Stem cell factor | pERK, pS6 Ribosome, pAKT, p-PLCg, |
| | p-Mek |
| Flt3 Ligand | pERK, p-Akt, p-Stat5, p-PLCg, p38, |
| | pNFkBp65, pMK2 |
| G-CSF | p-Stat-3, pStat-5.p-Akt, p-Erk, |
| | p-CREB (need to check CREB) |
| IL-3 | p-Stat5, p-Akt |

TABLE 5

| NK cells | Anti-phospho specific antibodies appropriate for stimulation |
|---|---|
| IL-18 | p-p38, pNFkBp65, p-Stat3, p-Stat6 |

TABLE 6

| P13-K | MAPK | JAK/STAT | NFkB | DNA damage | Apoptosis |
|---|---|---|---|---|---|
| Pathway | Pathway | Pathway | Pathway | Pathway | Pathway |
| p-Akt | p-Erk | p-STAT1 | p-IKKβ | p-Chk2 | c-PARP |
| p-GSK3β | p38 | P-STAT3 | p-IKKα | P-H2AX | c-Caspase 3 |
| p-Bad | p-S6 | p-STAT5 | IKBα | | c-Caspase 8 |
| p-Pras-40 | | | p-65 | | cytochrome C |
| mTOR | | | | | |
| p-S6 | | | | | |
| 4EBP1 | | | | | |

TABLE 7

| Modulator | Pathway Activated\hat{ } | Cell Sub-set |
|---|---|---|
| CD4O-L | PI3-K | B cells |
| | NFkB | |
| Baff/April | NFkB | B cells |
| Anti-μ | PI3-K | B cells |
| | MAP-K | |
| H₂O₂ | Phosphatases | All |
| IFNa | JAK/STAT | B cells |
| | | T cells |
| | | Monocytes |
| IFNγ | JAK/STAT | B cells |
| | | T cells |
| | | Monocytes |
| GM-CSF | JAK/STAT | Monocytes |
| | MAP-K | |
| | PI3-K | |
| G-CSF | JAK/STAT | Monocytes |
| | MAP-K | |
| | PI3-K | |
| IL-2 | JAK/STAT | T cells |
| IL-10 | JAK/STAT | B Cells |
| | | Monocytes |
| IL-6 | JAK/STAT | T Cells |
| | | Monocytes |
| IL-7 | JAK/STAT | |
| IL-4 | JAK/STAT | |
| IL-23 | JAK/STAT | |
| IL-27 | JAK/STAT | B cells |
| | | T cells |
| | | Monocytes |
| FLT3L | PI3K | Myeloid cells |
| | MAPK | |
| | JAK/STAT | |
| | p-CREB | |
| SCF | PI3K | Myeloid cells |
| | MAPK | |
| | JAK/STAT | |
| SDF1a | PI3K | Myeloid cells |
| | MAPK | |
| TNFa | p-IKKβ | T cells |
| | p-IKKα | Monocytes |
| | IKBαp-65 | |

\hat{ }Major pathways activated by these modulators.

TABLE 8

| Modulator | Published target | Manufacturer |
|---|---|---|
| JAK3 Inhibitor II | JAK3 | Calbiochem |
| Tyrene CR4 | JAK2 | Calbiochem |
| CP-690550 | JAK3 > JAK2 | ChemieTek |
| Cucurbitacin I | STAT3 | Calbiochem |
| A77 1726 | NFkB | Calbiochem |
| STAT3 Inhibitor VII | STAT3 | Calbiochem |
| JAK2 Inhibitor IV | JAK2 > JAK3 | Calbiochem |
| WH-P 154 | JAK3 | Tocris Bioscience |
| Pyridone 6 (JAK Inhibitor I) | Jak family kinases | Calbiochem |
| Jak3 Inhibitor VI | JAK3 | Calbiochem |
| LY294002 | PI3 Kinase | Calbiochem |
| U0126 | MEK1/MEK2 | Calbiochem |
| SB 203580 | P38 Kinase | Calbiochem |
| AG 490 | Jak family kinases | Calbiochem |

TABLE 9

| | | Lower Limit | Upper Limit |
|---|---|---|---|
| Num Cells | log10(IC50) | (95% Cl) | (95% CI) |
| 5 | −3.52 | −16.56 | 9.52 |
| 10 | −2.32 | −12.86 | 8.21 |
| 20 | −1.10 | −8.03 | 5.83 |
| 40 | −1.21 | −8.55 | 6.13 |
| 80 | −0.32 | −0.62 | −0.02 |
| 160 | −0.32 | −0.55 | −0.10 |
| 320 | −0.31 | −0.44 | −0.18 |
| 640 | −0.31 | −0.42 | −0.20 |
| 1280 | −0.32 | −0.38 | −0.25 |
| 2560 | −0.31 | −0.34 | −0.27 |

TABLE 10

| | | | Stat | Cell Types |
|---|---|---|---|---|
| | | | Family | Responsive |
| Ligand | Receptor | Jak-kinase | Members | to Ligand |
| IL-6 | IL-6Rα-gp130 | Jak1, | Stat1, | T cells, |
| | | Jak2, | Stat3 | monocytes, |
| | | Tyk2 | | neutrophils |
| G-CSF | G-CSFR | Jak2, | Stat3 | monocytes, |
| | | Tyk2 | | neutrophils, |
| | | | | myeloid |
| | | | | progenitors |
| GM-CSF | GM-CSFR + β_{c} | Jak2 | StatS | monocytes, |
| | | | | neutrophils, |
| | | | | myeloid |
| | | | | progenitors |
| IL-2 | IL-2Rα + | Jak1, | Stat5, | T cells |
| | IL-2Rβ + γ_{c} | Jak2, | Stat3 | |
| | | Jak3 | | |
| Tpo | TpoR (c-Mpl) | Tyk2, | StatS | myeloid |
| | | Jak2 | | progenitors |
| Epo | EpoR, ProlactinR | Jak2 | StatS | erythrocyte |
| | | | | progenitors |
| IFN-alpha | IFNAR1 + IFNAR2 | Jak1, | Stat1, | most cells |
| | | Tyk2 | Stat3, | |
| | | | Stat5 | |

Note:
p-Stats 1, 3, 5 all represent ‘validated’ nodes

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A method of analyzing the effect of a compound comprising: contacting a cell of interest with a compound of interest; analyzing activity of a gain-of-function mutation of a JAK/STAT pathway component in said cell; analyzing activity of a JAK/STAT regulatory protein in said cell; and correlating the activity of the JAK/STAT regulatory protein with the activity of the JAK/STAT pathway component.

2. The method of claim 2, wherein the gain-of-function mutation is a mutation in Jak-2.

3. The method of claim 3, wherein the mutation in Jak-2 is V617F.

4. The method of claim 1, wherein the JAK/STAT regulatory protein is SOCS3, Lnk, or SH2-B.

5. The method of claim 1, wherein the activity of a gain-of-function mutation of a JAK/STAT pathway component is analyzed by measuring phosphorylation of phospho-amino acid residues on Jak kinase, acytokine receptor, Stat, a PI3K-Akt pathway component or a Ras-Raf-Erk pathway component.

6. The method of claim 1, further comprising analyzing expression level of the JAK/STAT regulatory protein.

7. The method of claim 5, wherein the JAK/STAT regulatory protein is SOCS3, Lnk, or SH2-B.

8. The method of claim 1, wherein the cell of interest is a hematopoietic cell.

9. The method of claim 7, wherein the hematopoietic cell is involved in myeloproliferative disorders.

10. The method of claim 1, wherein the compound is a stimulator.

11. The method of claim 1, wherein the compound is an inhibitor of the JAK/STAT pathway.

12. The method of claim 1, further comprising administering a modulator.

13. The method of claim 10, wherein the modulator is a growth factor, cytokine, drug, immune modulator, ion, neurotransmitter, adhesion molecule, hormone, small molecule, inorganic compound, polynucleotide, antibody, natural compound, lectin, lactone, chemotherapeutic agent, biological response modifier, carbohydrate, protease, free radical, complex and undefined biologic composition, cellular secretion, glandular secretion, physiologic fluid, electromagnetic radiation, ultraviolet radiation, infrared radiation, particulate radiation, redox potential, pH modifier, the presence or absences of a nutrient, change in temperature, change in oxygen partial pressure, change in ion concentration or application of oxidative stress.

14. The method of claim 1, wherein the cell of interest is from a patient sample.

15. The method of claim 13, further comprising determining a clinical outcome based on the correlation of the activity of the JAK/STAT regulatory protein with the activity of the JAK/STAT pathway component.

16. The method of claim 14, further comprising determining a method of treatment of the patient based on the correlation of the activity of the JAK/STAT regulatory protein with the activity of the JAK/STAT pathway component.

17. The method of claim 1, further comprising analyzing an epigenetic change in the cell of interest.

18. The method of claim 16, wherein the epigenetic change is methylation or acetylation.

19. The method of claim 1, further comprising analyzing a microRNA change in the cell of interest.
